(19) United States
(12) Patent Application Publication
(10) Pub. No.: US 2026/0306841 A1
(43) Pub. Date: Oct. 8, 2026

(54) MEDICAL DEVICE INSERTION WITH CONTOUR MAPPING

(71) Applicant: COCHLEAR LIMITED, Macquarie University, NSW (AU)

(72) Inventor: Peter GIBSON, Macquarie University (AU)

(21) Appl. No.: 19/482,421

(22) PCT Filed: May 2, 2024
(86) PCT No.: PCT/IB2024/054270

§ 371 Date: Nov. 7, 2025

Related U.S. Application Data
(60) Provisional application No. 63/465,143, filed on May 9, 2023.

(51) Int. Cl. A61N 1/36; A61B 34/10; A61B 34/20; A61B 34/30; A61N 1/05

(52) U.S. Cl. CPC A61N 1/36039; A61B 34/10; A61N 1/0541; A61B 2034/105; A61B 2034/2065; A61B 2034/302

(57) ABSTRACT

A method, comprising advancing, as part of an implantation procedure into a human, at least a first portion of an electrode array into a cavity in a human during a first temporal period, providing information to a computer system, the provided information including spatial based data relating to the electrode array during the implantation procedure of the electrode array into the human, receiving information based on an evaluation by the computer system of the provided information, the evaluation having used the spatial based data to estimate a feature of the cavity, the received information being an indication of proximity between the electrode array and the feature of the cavity and at least one of completing the implantation procedure including leaving the electrode array at its current location based on the received information or moving the electrode array based on the received information.

### CROSS-REFERENCE TO RELATED APPLICATIONS

This application claims priority to U.S. Provisional Application No. 63/465,143, entitled MEDICAL DEVICE INSERTION WITH CONTOUR MAPPING, filed on May 9, 2023, naming Peter GIBSON as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In accordance with an exemplary embodiment, there is a method, comprising advancing, as part of an implantation procedure into a human, at least a first portion of an electrode array into a cavity in a human during a first temporal period, providing information to a computer system, the provided information including spatial based data relating to the electrode array during the implantation procedure of the electrode array into the human, receiving information based on an evaluation by the computer system of the provided information, the evaluation having used the spatial based data to estimate a feature of the cavity, the received information being an indication of proximity between the electrode array and the feature of the cavity and at least one of completing the implantation procedure including leaving the electrode array at its current location based on the received information or moving the electrode array based on the received information.

In an embodiment, there is a non-transitory computer readable medium having recorded thereon, a computer program for executing at least a portion of a method, the computer program including code for receiving input based on at least one of (i) imaging of a cavity of a human and at least a portion of a medical device in the cavity during a first temporal period or (ii) electrical measurements taken by the at least a portion of the medical device in the cavity during the first temporal period, code for automatically analyzing the received input to develop data indicative of an estimated boundary of the cavity and at least one of:
(a) code for determining a position of at least another portion of the medical device relative to the estimated boundary based on the received input; or
(b) code for receiving second input based on at least one of (i) second imaging of the cavity of the human and the at least another portion of the medical device or another medical device in the cavity during a second temporal period following the first temporal period or (ii) electrical measurements taken by the at least another portion of the medical device or the another medical device in the cavity during the second temporal period and code for determining the position of the at least another portion of the medical device or the another medical device relative to the estimated boundary based on one or both of the received input or the received second input.

In an embodiment, there is a method, comprising obtaining a model of a surface bounding a cavity inside a human during a medical device implantation procedure and at least one of confirming a position or repositioning the medical device based on the model of the surface.

In an embodiment, there is an apparatus, comprising a cochlear implant electrode array insertion device, wherein the device includes an input component configured to receive data based on data related to an insertion process of a cochlear implant electrode array, the received data being received in real time relative to the process and at least one of an output component configured to provide output to a user regarding an action to take with respect to implanting the array in a human or an actuator configured to move the array relative to the human in an automated manner, wherein the device at least one of (i) includes non-transitory logic or (ii) has access to non-transitory logic and/or results of analysis of non-transitory logic, wherein the non-transitory logic determines a distance and/or a location of a modiolus wall from one or more electrodes of the electrode array, the device is configured to at least one of provide output to the user indicative of the distance and/or location of the modiolus wall or control the actuator based on the determined distance and/or location of the modiolus wall.

In an embodiment, there is a method, comprising obtaining data indicative of a plurality of different spatial locations of a first portion(s) of a medical device, the plurality of different spatial locations being locations during an insertion process of the first portion(s) into a human, analyzing the data and at least one of:
(i) determining, based on the analysis, a location of a surface of a cavity in the human; or
(ii) determining, based on the analysis, a spatial relationship between the surface of the cavity and a second portion, the second portion being one of another portion of the medical device located away from the first portion(s) of the medical device or a portion of another medical device.

In an embodiment, there is a cochlear implant electrode array insertion robot, including a digital and/or analog input jack configured to receive digital and/or analog signals related to an insertion process of a cochlear implant electrode array, the received signals being received in real time relative to the process and at least one of an audio-visual device configured to provide output to a user regarding an action to take with respect to implanting the array in a human or an actuator configured to move the array relative to the human in an automated manner, wherein the device at least one of (i) includes non-transitory logic or (ii) has access to non-transitory logic and/or results of analysis of non-transitory logic, wherein the non-transitory logic determines a distance and/or a location of a modiolus wall from one or more electrodes of the electrode array, the device is configured to at least one of:
provide output to the user indicative of the distance and/or location of the modiolus wall; or
control the actuator based on the determined distance and/or location of the modiolus wall.

### BRIEF DESCRIPTION OF THE DRAWINGS

Embodiments are described below with reference to the attached drawings, in which:

FIGS. 1A-1C are views of exemplary prosthetic devices;

FIG. 2 is a side view of an embodiment of an insertion guide for implanting a cochlear implant electrode assembly such as the electrode assembly illustrated in FIG. 1;

FIGS. 3A and 3B are side and perspective views of an electrode assembly extended out of an embodiment of an insertion sheath of the insertion guide illustrated in FIG. 2;

FIGS. 4A-4E are simplified side views depicting the position and orientation of a cochlear implant electrode assembly insertion guide tube relative to the cochlea at each of a series of successive moments during an exemplary implantation of the electrode assembly into the cochlea;

FIGS. 4F-4I show side views depicting position and orientation of a cochlear implant electrode array;

FIG. 5A is a side view of a perimodiolar electrode assembly partially extended out of a conventional insertion guide tube showing how the assembly may twist while in the guide tube;

FIG. 6A is a cross-sectional view of a conventional electrode assembly;

FIG. 6B is a cross-sectional view of the conventional electrode assembly of FIG. 6C positioned in the insertion guide tube;

FIGS. 7A to 7D show views relating to electrode array position and orientation;

FIG. 8A-8C show views of respective different exemplary medical devices;

FIGS. 8D-8G show views relating to probe positioning;

FIGS. 8H-8K, 9A-9D and 11A-11D show views relating to electrode array positioning;

FIG. 8L and FIG. 9E shows views relating to trajectories;

FIG. 8M is an exemplary electrode array;

FIG. 10 shows a view showing a plurality of trajectories;

FIGS. 12-15 show exemplary flowcharts;

FIG. 16 shows a schematic of a device; and

FIGS. 17-38 present some exemplary embodiments of hardware for implementing some of the teachings detailed herein.

### DETAILED DESCRIPTION

Merely for ease of description, the techniques presented herein are sometimes described herein with reference to an illustrative medical device, namely a cochlear stimulator, and in other instances, a cochlear implant. However, it is to be appreciated that the techniques presented herein may also be used with a variety of other medical devices that, while providing a wide range of therapeutic benefits to recipients, patients, or other users, may benefit from setting changes based on the location of the medical device. For example, the techniques presented herein may be used with other hearing prostheses, including acoustic hearing aids, bone conduction devices, middle ear auditory prostheses, direct acoustic stimulators, other electrically simulating auditory prostheses (e.g., auditory brain stimulators), etc. Some embodiments include the utilization of the teachings herein to treat an inner ear of a recipient that has and/or utilizes one or more of these devices. The techniques presented herein may also be used with vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc. In further embodiments, the techniques presented herein may be used with air purifiers or air sensors (e.g., automatically adjust depending on environment), hospital beds, identification (ID) badges/bands, or other hospital equipment or instruments.

The teachings detailed herein can be implemented in sensory prostheses, such as hearing implants specifically, and neural stimulation devices in general. Other types of sensory prostheses can include retinal implants. Accordingly, any teaching herein with respect to a sensory prosthesis corresponds to a disclosure of utilizing those teachings in/with a hearing implant and in/with a retinal implant, unless otherwise specified, providing the art enables such. Moreover, with respect to any teachings herein, such corresponds to a disclosure of utilizing those teachings with all of or parts of a cochlear implant, cochlear stimulator, a bone conduction device (active and passive transcutaneous bone conduction devices, and percutaneous bone conduction devices) and a middle ear implant, providing that the art enables such, unless otherwise noted. To be clear, any teaching herein with respect to a specific sensory prosthesis corresponds to a disclosure of utilizing those teachings in/with any of the aforementioned hearing prostheses, and vice versa. Corollary to this is at least some teachings detailed herein can be implemented in somatosensory implants and/or chemosensory implants. Accordingly, any teaching herein with respect to a sensory prosthesis corresponds to a disclosure of utilizing those teachings with/in a somatosensory implant and/or a chemosensory implant.

Thus, merely for ease of description, the first illustrative medical device is a hearing prosthesis. Any techniques presented herein described for one type of hearing prosthesis or any other device disclosed herein corresponds to a disclosure of another embodiment of using such teaching with another device (and/or another type of hearing device including other types of bone conduction devices (active transcutaneous and/or passive transcutaneous), middle ear auditory prostheses (particularly, the EM vibrator/actuator thereof), direct acoustic stimulators), etc. The techniques presented herein can be used with implantable/implanted microphones (where such is a transducer that receives vibrations and outputs an electrical signal (effectively, the reverse of an EM actuator), whether or not used as part of a hearing prosthesis (e.g., a body noise or other monitor, whether or not it is part of a hearing prosthesis) and/or external microphones. The techniques presented herein can also be used with vestibular devices (e.g., vestibular implants), sensors, seizure devices (e.g., devices for monitoring and/or treating epileptic events, where applicable), and thus any disclosure herein is a disclosure of utilizing such devices with the teachings herein (and vice versa), providing that the art enables such. The teachings herein can also be used with conventional hearing devices, such as telephones and ear bud devices connected MP3 players or smart phones or other types of devices that can provide audio signal output, that use an EM transducer. Indeed, the teachings herein can be used with specialized communication devices, such as military communication devices, factory floor communication devices, professional sports communication devices, etc.

By way of example, any of the technologies detailed herein which are associated with components that are implanted in a recipient can be combined with information delivery technologies disclosed herein, such as for example, devices that evoke a hearing percept, to convey information to the recipient. By way of example only and not by way of limitation, a sleep apnea implanted device can be combined with a device that can evoke a hearing percept so as to provide information to a recipient, such as status information, etc. In this regard, the various sensors detailed herein and the various output devices detailed herein can be combined with such a non-sensory prosthesis or any other nonsensory prosthesis that includes implantable components so as to enable a user interface, as will be described herein, that enables information to be conveyed to the recipient, which information is associated with the implant.

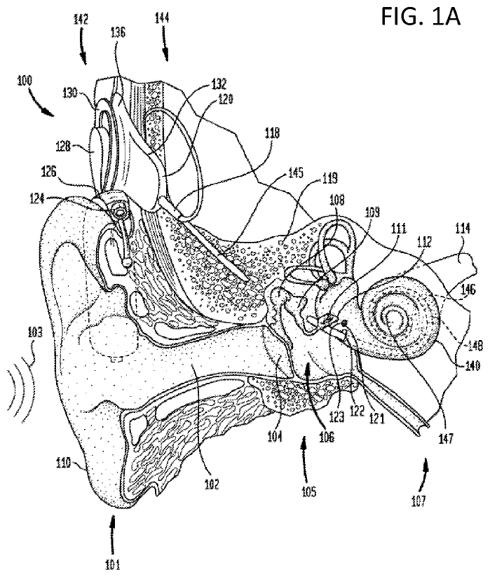

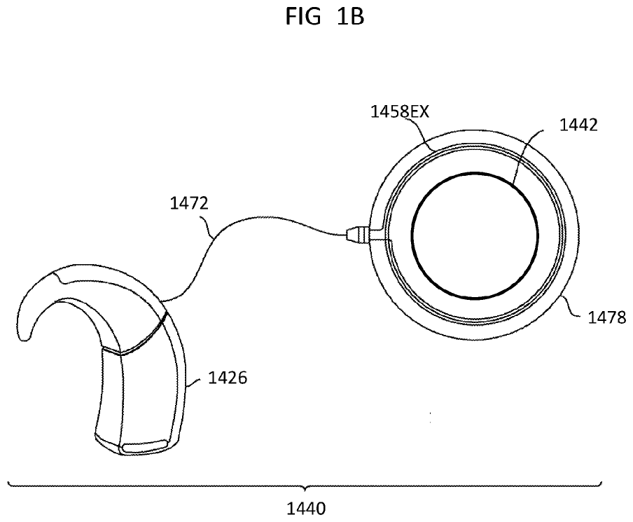

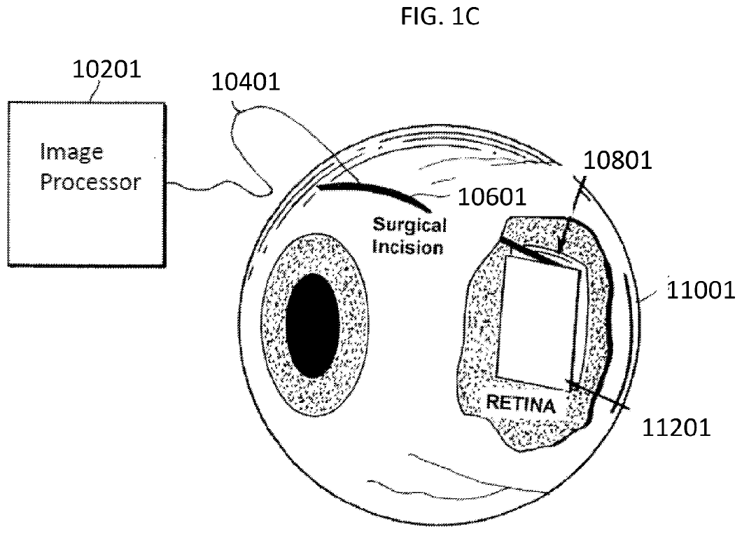

FIG. 1A is a perspective view of an exemplary cochlear implant 100 implanted in a recipient having an outer ear 101, a middle ear 105, and an inner ear 107. In a fully functional ear, outer ear 101 comprises an auricle 110 and an ear canal 102. Acoustic pressure or sound waves 103 are collected by auricle 110 and channeled into and through ear canal 102. Disposed across the distal end of ear canal 102 is a tympanic membrane 104 that vibrates in response to sound waves 103. This vibration is coupled to oval window or fenestra ovalis 112 through the three bones of the middle ear 105, collectively referred to as the ossicles 106, and comprising the malleus 108, the incus 109, and the stapes 111. Ossicles 106 filter and amplify the vibrations delivered by tympanic membrane 104, causing oval window 112 to articulate, or vibrate. This vibration sets up waves of fluid motion of the perilymph within cochlea 140. Such fluid motion, in turn, activates hair cells (not shown) inside the cochlea which in turn causes nerve impulses to be generated which are transferred through spiral ganglion cells (not shown) and auditory nerve 114 to the brain (also not shown) where they are perceived as sound.

The exemplary cochlear implant illustrated in FIG. 1A is a partially implanted stimulating medical device. Specifically, cochlear implant 100 comprises external components 142 attached to the body of the recipient, and internal or implantable components 144 implanted in the recipient. External components 142 typically comprise one or more sound input elements for detecting sound, such as microphone 124, a sound processor (not shown), and a power source (not shown). Collectively, these components are housed in a behind-the-ear (BTE) device 126 in the example depicted in FIG. 1A. External components 142 also include a transmitter unit 128 comprising an external coil 130 of a transcutaneous energy transfer (TET) system. Sound processor 126 processes the output of microphone 124 and generates encoded stimulation data signals which are provided to external coil 130.

Internal components 144 comprise an internal receiver unit 132 including a coil 136 of the TET system, a stimulator unit 120, and an elongate stimulating lead assembly 118. Internal receiver unit 132 and stimulator unit 120 are hermetically sealed within a biocompatible housing commonly referred to as a stimulator/receiver unit. Internal coil 136 of receiver unit 132 receives power and stimulation data from external coil 130. Stimulating lead assembly 118 has a proximal end connected to stimulator unit 120, and extends through mastoid bone 119. Lead assembly 118 has a distal region, referred to as electrode assembly 145, a portion of which is implanted in cochlea 140.

Electrode assembly 145 can be inserted into cochlea 140 via a cochleostomy 122, or through round window 121, oval window 112, promontory 123, or an opening in an apical turn 147 of cochlea 140. Integrated in electrode assembly 145 is an array 146 of longitudinally-aligned and distally extending electrode contacts 148 for stimulating the cochlea by delivering electrical, optical, or some other form of energy. Stimulator unit 120 generates stimulation signals each of which is delivered by a specific electrode contact 148 to cochlea 140, thereby stimulating auditory nerve 114.

FIG. 1B depicts an exemplary external component 1440. External component 1440 can correspond to external component 142 of the system 10 (it can also represent other body worn devices herein/devices that are used with implanted portions). As can be seen, external component 1440 includes a behind-the-ear (BTE) device 1426 which is connected via cable 1472 to an exemplary headpiece 1478 including an external inductance coil 1458EX, corresponding to the external coil of FIG. 1. As illustrated, the external component 1440 comprises the headpiece 1478 that includes the coil 1458EX and a magnet 1442. This magnet 1442 interacts with the implanted magnet (or implanted magnetic material) of the implantable component to hold the headpiece 1478 against the skin of the recipient. In an exemplary embodiment, the external component 1440 is configured to transmit and/or receive magnetic data and/or transmit power transcutaneously via coil 1458EX to the implantable component, which includes an inductance coil. The coil 1458X is electrically coupled to BTE device 1426 via cable 1472. BTE device 1426 may include, for example, at least some of the components of the external devices/components described herein.

FIG. 1C presents an exemplary embodiment of a neural prosthesis in general, and a retinal prosthesis and an environment of use thereof, in particular, the components of which can be used in whole or in part, in some of the teachings herein. In some embodiments of a retinal prosthesis, a retinal prosthesis sensor-stimulator 10801 is positioned proximate the retina 11001. In an exemplary embodiment, photons entering the eye are absorbed by a microelectronic array of the sensor-stimulator 10801 that is hybridized to a glass piece 11201 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 10201 is in signal communication with the sensor-stimulator 10801 via cable 10401 which extends through surgical incision 00601 through the eye wall (although in other embodiments, the image processor 10201 is in wireless communication with the sensor-stimulator 10801). The image processor 10201 processes the input into the sensor-stimulator 10801 and provides control signals back to the sensor-stimulator 10801 so the device can provide processed output to the optic nerve. That said, in an alternate embodiment, the processing is executed by a component proximate with or integrated with the sensor-stimulator 10801. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 10801 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of an array for a hearing prosthesis corresponds to a disclosure of an array for a retinal prosthesis. Any disclosure herein of a method of using or operating or otherwise working with a hearing prosthesis herein corresponds to a disclosure of using or operating or otherwise working with a retinal prosthesis in an analogous manner.

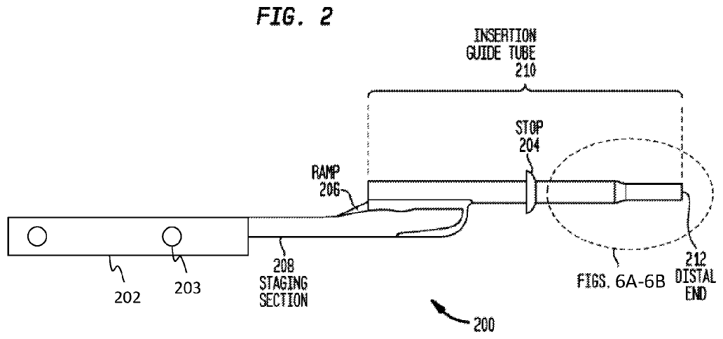

Electrode assembly 145 may be inserted into cochlea 140 with the use of an insertion guide. FIG. 2 is a side view of an embodiment of an insertion guide for implanting an elongate electrode assembly generally represented by electrode assembly 145 into a mammalian cochlea, represented by cochlea 140. The illustrative insertion guide, referred to herein as insertion guide 200, includes an elongate insertion guide tube 210 configured to be inserted into cochlea 140 and having a distal end 212 from which an electrode assembly is deployed. Insertion guide tube 210 has a radially-extending stop 204 that may be utilized to determine or otherwise control the depth to which insertion guide tube 210 is inserted into cochlea 140.

Insertion guide tube 210 is mounted on a distal region of an elongate staging section 208 on which the electrode assembly is positioned prior to implantation. A robotic arm adapter 202 is mounted to a proximal end of staging section 208 to facilitate attachment of the guide to a robot, which adapter includes through holes 203 through which bolts can be passed so as to bolt the guide 200 to a robotic arm, as will be detailed below. During use, electrode assembly 145 is advanced from staging section 208 to insertion guide tube 210 via ramp 206. After insertion guide tube 210 is inserted to the appropriate depth in cochlea 140, electrode assembly 145 is advanced through the guide tube to exit distal end 212 as described further below.

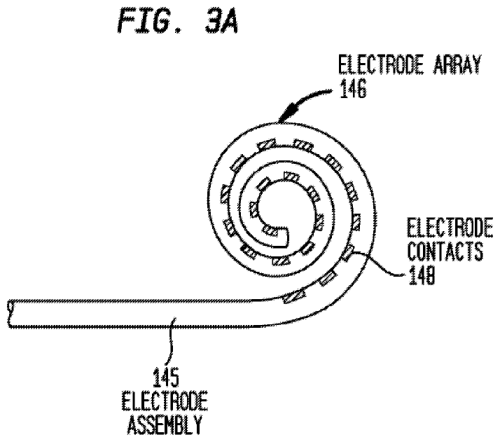

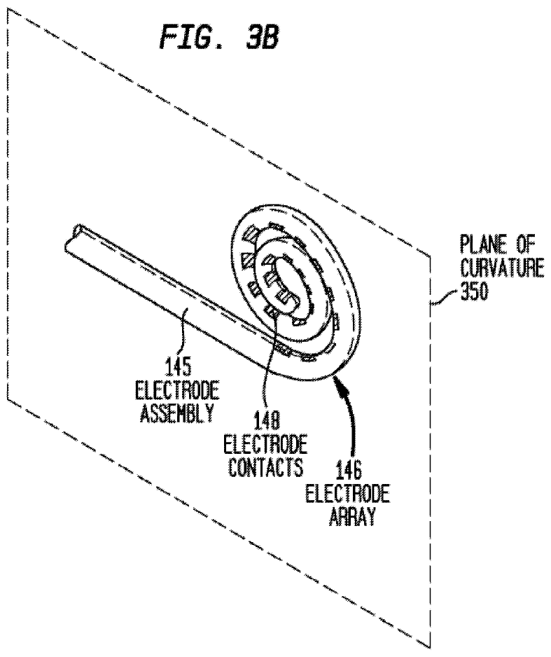

FIGS. 3A and 3B are side and perspective views, respectively, of representative electrode assembly 145. As noted, electrode assembly 145 comprises an electrode array 146 of electrode contacts 148. Electrode assembly 145 is configured to place electrode contacts 148 in close proximity to the ganglion cells in the modiolus. Such an electrode assembly, commonly referred to as a perimodiolar electrode assembly, is manufactured in a curved configuration as depicted in FIGS. 3A and 3B. When free of the restraint of a stylet or insertion guide tube, electrode assembly 145 takes on a curved configuration due to it being manufactured with a bias to curve, so that it is able to conform to the curved interior of cochlea 140. As shown in FIG. 3B, when not in cochlea 140, electrode assembly 145 generally resides in a plane 350 as it returns to its curved configuration. That said, it is noted that embodiments of the insertion guides detailed herein and/or variations thereof can be applicable to a so-called straight electrode array, which electrode array does not curl after being free of a stylet or insertion guide tube etc., but instead remains straight.

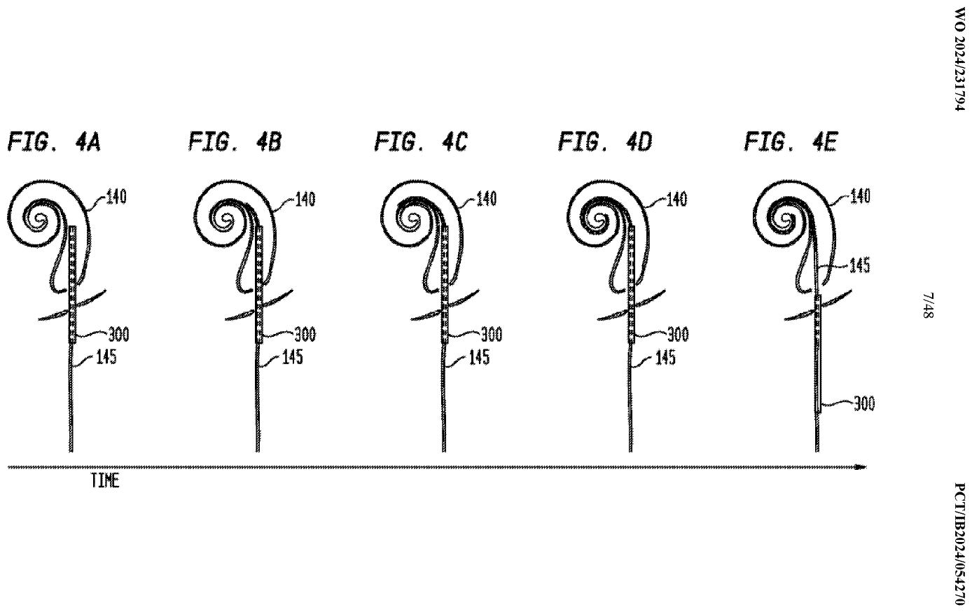

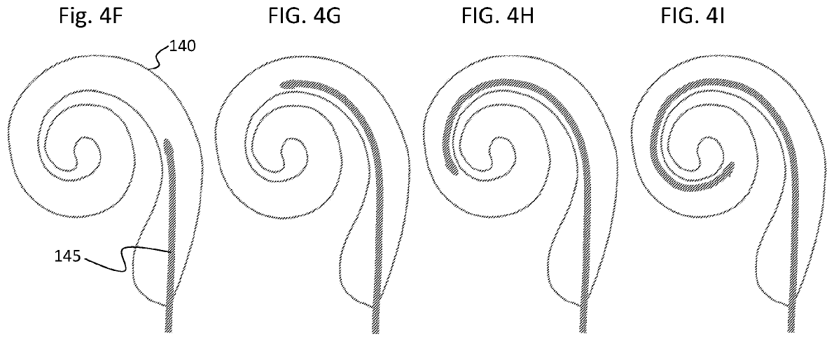

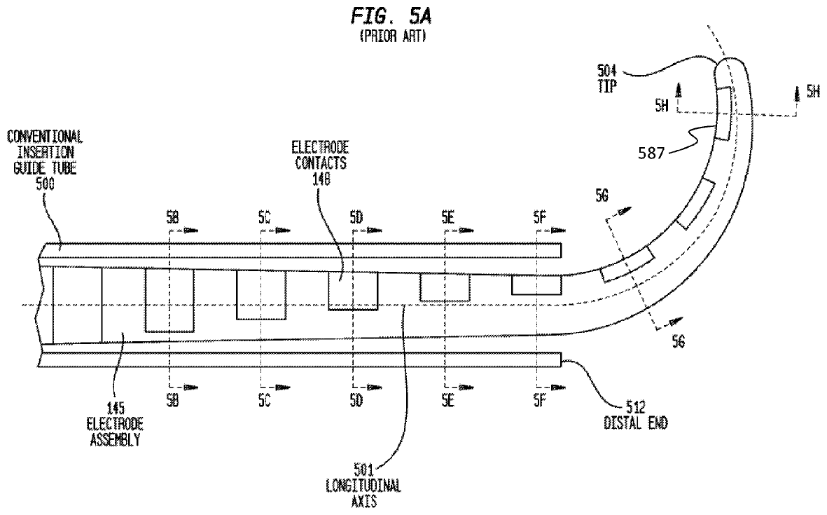

FIGS. 4A-4E are a series of side-views showing consecutive exemplary events that occur in an exemplary implantation of electrode assembly 145 into cochlea 140. Initially, electrode assembly 145 and insertion guide tube 310 are assembled. For example, electrode assembly 145 is inserted (slidingly or otherwise) into a lumen of insertion guide tube 300. The combined arrangement is then inserted to a predetermined depth into cochlea 140, as illustrated in FIG. 4A. Typically, such an introduction to cochlea 140 is achieved via cochleostomy 122 (FIG. 1) or through round window 121 or oval window 112. In the exemplary implantation shown in FIG. 4A, the combined arrangement of electrode assembly 145 and insertion guide tube 300 is inserted to approximately the first turn of cochlea 140.

As shown in FIG. 4A, the combined arrangement of insertion guide tube 300 and electrode assembly 145 is substantially straight. This is due in part to the rigidity of insertion guide tube 300 relative to the bias force applied to the interior wall of the guide tube by pre-curved electrode assembly 145. This prevents insertion guide tube 300 from bending or curving in response to forces applied by electrode assembly 145, thus enabling the electrode assembly to be held straight, as will be detailed below.

As noted, electrode assembly 145 is biased to curl and will do so in the absence of forces applied thereto to maintain the straightness. That is, electrode assembly 145 has a memory that causes it to adopt a curved configuration in the absence of external forces. As a result, when electrode assembly 145 is retained in a straight orientation in guide tube 300, the guide tube prevents the electrode assembly from returning to its pre-curved configuration. This induces stress in electrode assembly 145. Pre-curved electrode assembly 145 will tend to twist in insertion guide tube 300 to reduce the induced stress. In the embodiment configured to be implanted in scala tympani of the cochlea, electrode assembly 145 is pre-curved to have a radius of curvature that approximates the curvature of medial side of the scala tympani of the cochlea. Such embodiments of the electrode assembly are referred to as a perimodiolar electrode assembly, and this position within cochlea 140 is commonly referred to as the perimodiolar position. In some embodiments, placing electrode contacts in the perimodiolar position provides utility with respect to the specificity of electrical stimulation, and can reduce the requisite current levels thereby reducing power consumption.

As shown in FIGS. 4B-4D, electrode assembly 145 may be continually advanced through insertion guide tube 300 while the insertion sheath is maintained in a substantially stationary position. This causes the distal end of electrode assembly 145 to extend from the distal end of insertion guide tube 300. As it does so, the illustrative embodiment of electrode assembly 145 bends or curves to attain a perimodiolar position, as shown in FIGS. 4B-4D, owing to its bias (memory) to curve. Once electrode assembly 145 is located at the desired depth in the scala tympani, insertion guide tube 300 is removed from cochlea 140 while electrode assembly 145 is maintained in a stationary position. This is illustrated in FIG. 4E.

In an embodiment, the goal of the insertion process is to achieve the insertion regime seen in FIGS. 4A-4E, at least for a pre-curved array.

And note that embodiments include insertion of electrode arrays without a guide tube or a guide device whatsoever. FIGS. 4F to 4I show such an insertion. These figures present a series of diagrams that show an optimal insertion of a pre-curved perimodiolar electrode array at different points during insertion. In this example the electrode takes an optimal path around the modiolus, maintaining proximity to the modiolus throughout the insertion.

Returning to FIGS. 3A-3B, perimodiolar electrode assembly 145 is pre-curved in a direction that results in electrode contacts 148 being located on the interior of the curved assembly, as this causes the electrode contacts to face the modiolus when the electrode assembly is implanted in or adjacent to cochlea 140. Insertion guide tube 500 retains electrode assembly 145 in a substantially straight configuration, thereby preventing the assembly from taking on the configuration shown in FIG. 3B.

FIG. 5A is a side view of perimodiolar electrode assembly 145 partially extended out of a conventional insertion guide tube 500, showing how the assembly may twist while in the guide tube.

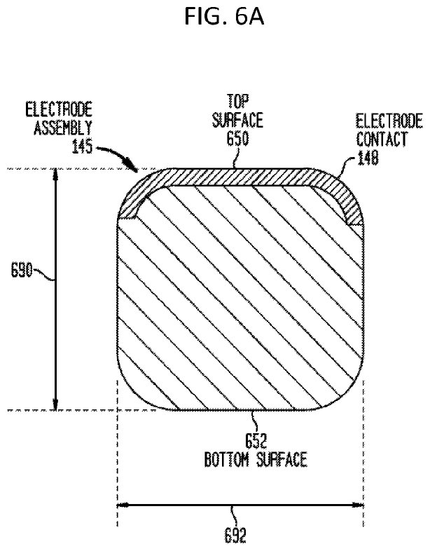

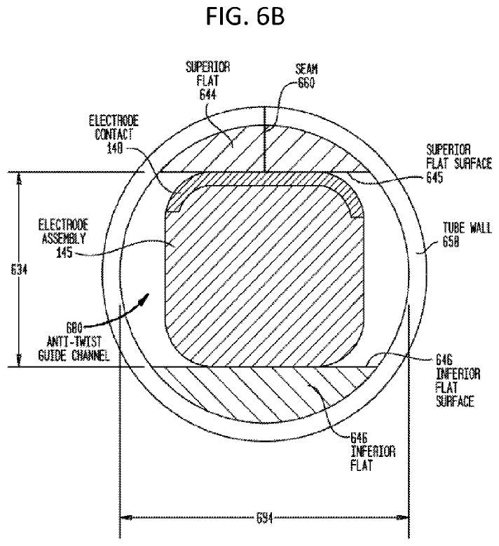

As shown in FIG. 6A, electrode assembly 145 has a rectangular cross-sectional shape, with the surface formed in part by the surface of the electrode contact, referred to herein as top surface 650, and the opposing surface, referred to herein as bottom surface 652, are substantially planar. In other embodiments, the array is circular or ovaloid in cross-section.

FIG. 6B shows a cross-section of the insertion guide tube and the electrode assembly 145. Tube wall 658 has surfaces 644 and 646 which extend radially inward to form an anti-twist guide channel 680. Specifically, a superior flat 644 provides a substantially planar lumen surface along the length of a section of the tube. Superior flat 644 has a surface that is substantially planar and which therefore conforms with the substantially planar top surface 650 of electrode assembly 145. Similarly, inferior flat 646 has a surface that is substantially planar which conforms with the substantially planar bottom surface 652 of electrode assembly 145. As shown in FIG. 6D, when a distal region of electrode assembly 145 is located in anti-twist section 620, the surfaces of superior flat 644 and inferior flat 646 are in physical contact with top surface 650 and bottom surface 652, respectively, of the electrode assembly. This prevents the electrode assembly from curving.

With the above as context, embodiments include cochlear implant electrode array implantation techniques that include providing input or otherwise guidance/recommendations to a surgeon or other professional who is inserting the array into the cochlea, in real time, while the healthcare professional is inserting the array into the cochlea.

It is briefly noted that embodiments are not limited to insertion of an electrode array into a cochlea. Any disclosure herein related to an electrode array corresponds to another alternate disclosure of an insertion of another device, into a cochlea, for the purposes of textual economy, providing that such is utilitarian value providing that the art enables such. Corollary to this is that any disclosure herein related to a cochlea corresponds to an alternate disclosure of another cavity within a human, provided that the art enables such and providing that there is utilitarian value with respect to applications of the teachings detailed herein there to.

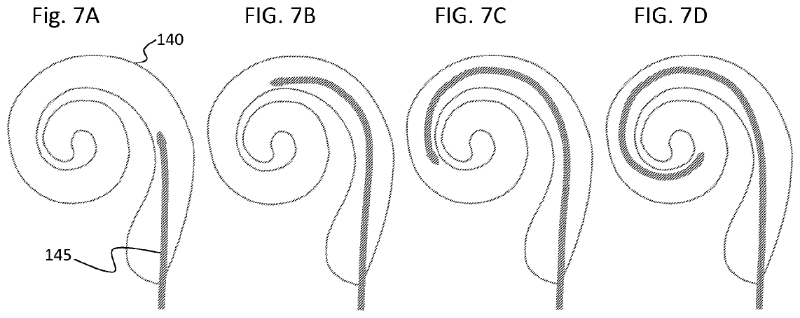

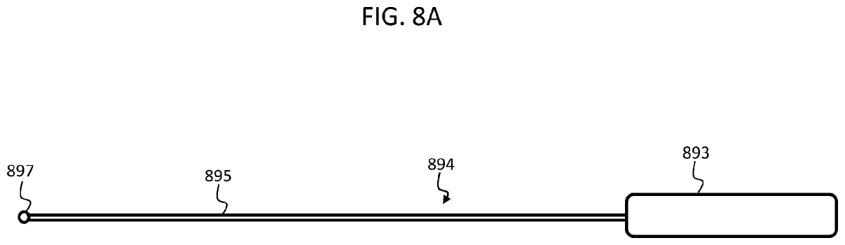

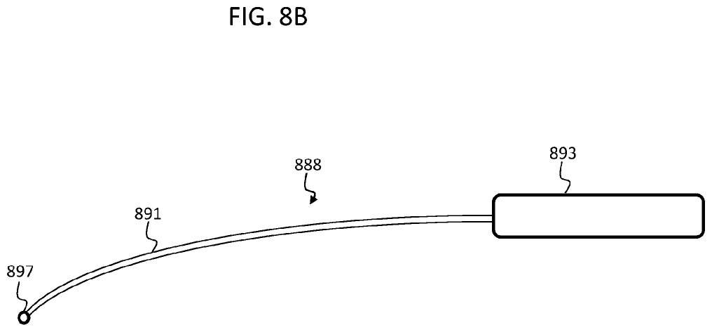

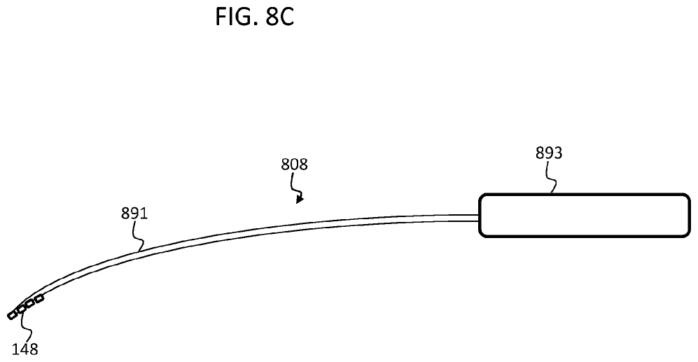

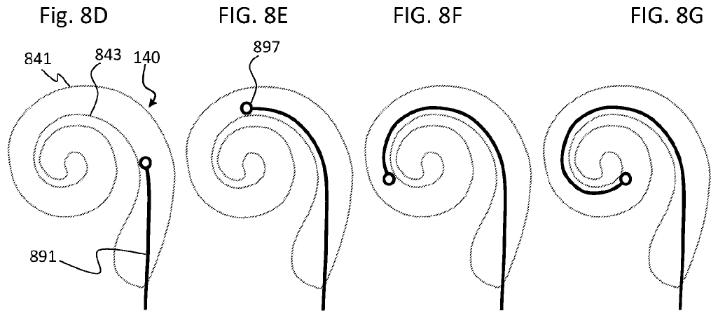

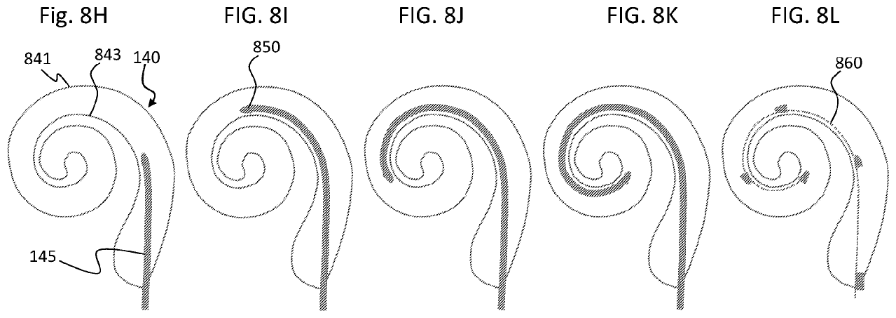

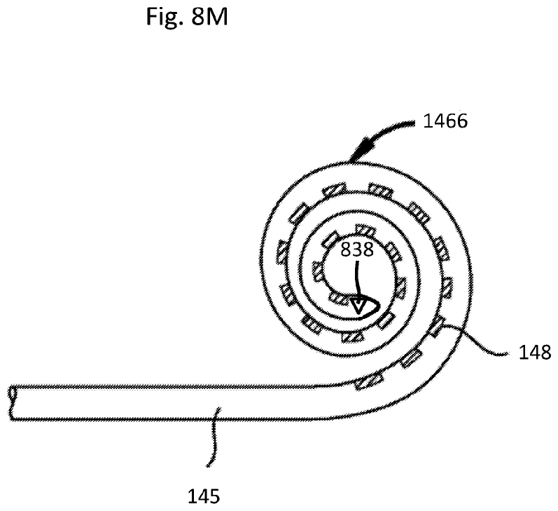

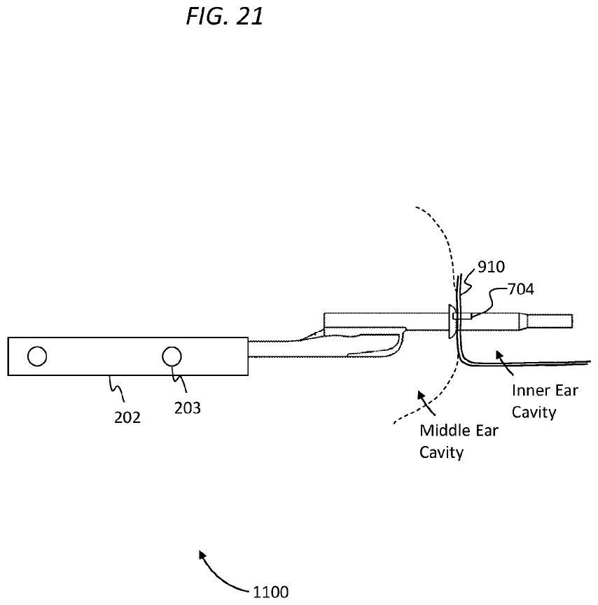

Pre-curved perimodiolar electrode arrays are intended to follow a trajectory around the modiolus, staying close to the modiolus wall and away from the delicate structures at the lateral wall of the otic capsule. FIGS. 4A-4I show insertion of the array following such a trajectory. A design intent of at least some perimodiolar electrode arrays is to be positioned as close as possible (herein, proximate) to the modiolus wall of the cochlea. However, the precise contour/position of the modiolus wall in a patient undergoing cochlear implant surgery is relatively unknown prior to surgery and even during surgery. This makes it difficult to estimate if a perimodiolar electrode array is positioned close to the modiolus wall. This can be because the complex morphology of the inner ear and intervening structures obscure the contour/position of the modiolus wall in imaging. In contrast, the location/contour of the lateral wall of the otic capsule can be relatively well identified in pre-operative (the operation to implant the cochlear implant electrode array) imaging and/or imaging during the operation implanting of the cochlear implant. Also, the insertion of the array is typically done blindly vis-à-vis what is actually happening in the cochlea with respect to the array, literally and often figuratively. In at least some exemplary embodiments there is no imaging that occurs during insertion with respect to what is going on in the cochlea. Corollary to this is that tactile response during the insertion process is open to too much judgment and otherwise can be misleading. Indeed, in the case of a pre-curved electrode array being inserted with the use of an insertion tube or insertion sheath, the friction of the array in the sheath dominates what the surgeon feels, leading to, in some instances, tactile blindness. Various features, including natural features, associated with the cochlea, such as for example a scalloped wall of the cochlea duct into which the array is being inserted, the angle of insertion, the friction associated with the surgical opening into the cochlea from the middle ear or from outside the cochlea and other natural features of the cochlea that can vary from human to human and can result in the tactile response being non-utilitarian with respect to understanding what is exactly happening within the cochlea with respect to progression of the electrode array therein. Corollary to this is that even when tactile response occurs, and if that tactile response was perfectly translatable and/or understandable by the surgeon, or even with imaging or some artificial method of determining the placement and/or prose of the electrode array in the cochlea in real time, the surgeon still may not have a lot of guidance or any guidance for that matter as to what should happen next with respect to insertion action relative to the next insertion action. In this regard, should the surgeon or other healthcare professional continue to insert the electrode array further, or should the surgeon stop and potentially retract the array? Should the surgeon twist the array, etc.? No guidance is given. It is totally up to the subjective and personally experience-based understanding of the surgeon as to what should be done next. And in at least some exemplary scenarios of insertion, sometimes pre-curved electrode arrays follow a less-optimal path. This is seen in FIGS. 7A-7D. In this scenario, the mid section of the array does not follow the modiolus during insertion. Friction at the tip prevents forward movement of the tip which causes the mid-section of the array to flex outward toward the delicate structures of the lateral wall. If the mid-section of the array presses on the lateral wall there is potential to cause disturbance or trauma to the delicate structures on the lateral wall. FIG. 7D shows the final position of the electrode array, where the array is not as close to the modiolus as in FIG. 4I (FIG. 4I showing the more utilitarian placement of the array). The scenario shown in FIGS. 7A-7D can occur due to a higher than normal level of friction at the tip, which can prevent the tip from moving forward the same distance as the insertion movement. The friction can be due to an uneven surface along the modiolus wall by way of example and not by way of limitation. Histology shows that some modular walls exhibit a scalloped surface which can catch the tip. When this happens the mid-section of the electrode array flexes out until there is sufficient forward push from the tensioned array to overcome the friction of the tip on the modiolus. In some cases, the mid-section of the electrode array can move out far enough to press on the delicate structures of the lateral wall and have potential to cause some disturbance or trauma to those structures. This can be not good in some instances. If an array presses on the lateral wall, there can be forces upward toward the basilar membrane 888, as seen in FIG. 8, where F₁ is the force that results from the electrode array boing outward away from the modiolus wall, and Rᵣ is the reaction force owing to the curvature of the modiolus wall that drives the electrode array upwards towards the basilar membrane 888, with β being the angle relative to F₁ and the resulting force Rᵣ. (FIG. 8 comes from It is from a paper by Prof. J Thomas Roland of NYU. A Model for Cochlear Implant Electrode Insertion and Force Evaluation: Results with a New Electrode Design and Insertion Technique, Laryngoscope 115: August 2005. FIG. 21.)

Even when a surgeon or otherwise a healthcare professional has reason to believe or suspect or otherwise has knowledge that there is an existing placement and/or pose an electrode array that can be deleterious at the moment or could result in a deleterious scenario in the future, as mentioned above, the surgeon or otherwise the healthcare professional (hereinafter, reference will be made to the surgeon, but such corresponds to an alternate disclosure in the interest of textual economy to another type of healthcare professional) has no guidance as to what action to next take. That is, for example, the surgeon does not have any guidance regarding what to do should an electrode tip stop or slow progression such that the mid-section of the array starts to flex out toward the lateral wall. Should the surgeon keep pushing forward in the hope that the tip will start to move? Should the surgeon pull back to retract the tip from the point of high friction and then re-start to insert? Embodiments can provide utilitarian value with respect to providing guidance to the surgeon on a next maneuver (e.g., the likely “best” next maneuver, based on statistics—more on this below) to take in the circumstances that exist at the moment.

Embodiments include insertion actions where the electrode array is inserted in a slow and/or step-wise fashion with some forward and, in some instances, some backward movements, where the movements (forward and/or backward) might be intentional or unintentional. An unintentional movement could happen for example if an electrode array is released and the stored elastic energy in the electrode lead (the part outside the cochlea) causes a movement.

Embodiments include an algorithm to improve an estimation of the contour/position of the modiolus wall relative to the lateral wall for each individual, and such can be utilitarian in some embodiments, such as when this data could be used with other algorithms that can locate the position of a perimodiolar electrode array relative to the lateral wall and other structures of the human by way of example to assist surgeons in optimizing the position of a perimodiolar electrode array close to the modiolus. In contrast to the current state of technology which is to make an estimate of the modiolus wall by comparison of the outer wall and other anatomical features against an atlas or mathematical model built from cadaver samples which have been analyzed with micro-CT or histology to precisely locate the modiolus wall in the sample cases, the teachings herein can provide real-time data on the actual modiolus wall location of a specific individual without reference to other anatomical structures or landmarks (although such can be used in some embodiments). Anatomical landmarks and dimensions that are visible in the pre-operative imaging need not be used to scale and register against a specific patient image to estimate the contour and location of the modiolus wall relative to the known structures to develop a location of the modiolus wall.

Embodiments include devices, systems, and methods that can be used to improve, including optimize, electrode array insertion and/or placement/final positioning thereof close to the modiolus. The teachings herein can use any type of monitoring that can have utilitarian value vis-à-vis determining position of at least a portion of a medical device, such as a probe or an electrode array during insertion. Embodiments can use, for example only and not by way of limitation, imaging techniques such as x-ray, fluoroscopy, CT scans, MRI. Also, electrical techniques of estimating electrode position (whether as part of the electrode array or another medical device used to analyze the anatomical structure) in the scala such as described in PCT Patent Application Publication No. WO 2021/028824, or WO 2018/173010, or WO 2019/162837 or WO 2019/175764 or US Patent Application Publication No. 2022/0016416 or US Patent Application Publication No. 2018/0140829 or US Patent Application Publication No. 2018/0050196, to inventor Nicolas Pawsey, Published on Feb. 22, 2018 or U.S. Provisional Patent Application Ser. No. 63/277,253 and PCT/IB2022/060473.

FIG. 8A shows an exemplary medical device 894 that includes a handle 893, a flexible rod 895, and a spherical probe 897. In this exemplary embodiment, the medical device 894 is configured to be held by the handle 893 by a surgeon or other healthcare professional, and the probe and a portion of the rod 895 is inserted into an opening into a cavity in a human. The probe 897 is made of a material that is readily imaged in an X-ray or a CT scan, or other types of imaging techniques detailed herein. In an exemplary embodiment, the probe 897 is a solid sphere of radiopaque material, such as barium. FIG. 8B shows another medical device 888, where the rod 891 is pre-curved as shown in a manner concomitant with a pre-curved cochlear implant electrode array/perimodiolar array. In an embodiment, sphere 897 is made of barium. In an embodiment, sphere 897 can be a material that gives off radiation or reacts to fluoroscopy, etc. Any device, system, and/or method that can enhance imaging can be used in some embodiments. Corollary to this is that FIG. 8C shows another exemplary medical device 808 that instead of having a probe of radiopaque material, the medical device 808 includes a plurality of electrical contacts 148 located at the end of the flexible rod as shown. In an exemplary embodiment, these contacts are connected to leads (not shown) which extended the handle 893, and the handle 893 has a jack that allows for connection to a computer system to enable electrical measurements to be made when the contacts 148 are located inside a cavity in a human so as to obtain spatial data associated with a wall of the cavity. Some additional details of this will be discussed below. In an embodiment, the sphere 897 can be Peek and can have a barium ball at a center (with a known distance from the outside of the sphere to the center of the barium ball so that this can be scaled/that the center of the ball can be used as a reference to determine the distance from the center to the outside of the sphere, and thus the location of the modiolus wall). The device 808 can have utilitarian value of the surgeon is comfortable with inserting two different devices into the cavity. Alternatively and/or in addition to this, an ultrasound sensor can be provided in the tip to provide distancing to the wall. Also, this can be utilitarian for a person with a compromised lumen due to disease. Also, in an embodiment, the probe could have contacts spaced radially around the ball, and this can be used to map the volume of the cavity. In an embodiment, the barium body (e.g., ball) can be located in a tip of the electrode array, such as within 0.5, 1, 1.5 or 2 mm from the most distal end thereof.

In an embodiment, as the probe is inserted into the cavity (e.g., a cochlea), the probe tracks around the scala tympani close to the modiolus. In an exemplary embodiment, if the position of the probe 897 is recorded one or more times or otherwise repeatedly during insertion, and in some embodiments, if the insertion depth is estimated at the same time, embodiments can include utilizing such to trace out the contour of the modiolus. In an embodiment, the probe 897 is covered with a low friction material or otherwise is lubricated so that the probe 897 slides along the modiolus wall as the probe is inserted into the cochlea.

FIGS. 8D-8G are a series of diagrams showing the probe 897 at different points during insertion of a pre-curved perimodiolar flexible rod, or any other component of suitable flexibility that can enable the teachings herein, but again, the teachings herein can be used with a straight flexible rod. The probe 897 tracks around the modiolus wall 843 and the position of the probe is determined (e.g., recorded) at several different temporal locations as the probe is advanced into the cavity (here, a scala of the cochlea). By obtaining a record of the probe at the 4 insertion points shown (by way of example), a contour of the modiolus wall 843 is developed, here, virtually/by way of a dataset, can be developed.

But embodiments can also use an electrode array as part of a process to model the contour of the wall 843. In an embodiment, as a pre-curved array (or a straight array in some other embodiments) is inserted, the tip and/or a most forward electrode tracks around the scala tympani close to the modiolus. Hereinafter, this is sometimes referred to as the distal end portion—this can include the tip and/or 1, 2, 3, or 4 electrodes from the tip (the four most distal electrodes). Embodiments will be referred to herein variously as the tip portion, the tip, the forward electrode, the forward portion, etc. Reference to one herein corresponds to an alternate disclosure of any of these others (e.g., any one or more or all of the most distal 1, 2, 3, or 4 electrodes, for example) in the interests of textual economy. In an exemplary embodiment, if the position of the tip electrode (i.e., the most distal electrode) by way of example is recorded one or more times or otherwise repeatedly during insertion, and in some embodiments, if the insertion depth is estimated at the same time, embodiments can include utilizing such to trace out the contour of the modiolus as shown in FIGS. 8H-8L.

FIGS. 8H-8L are a series of diagrams showing the electrode array at different points during insertion of a pre-curved perimodiolar electrode array, but again, the teachings herein can be used for a straight array. The tip portion 850 and in this embodiment, the most distal contact (electrode contact 587 of FIG. 5A) tracks around the modiolus wall 843 and the position of the tip electrode contact (most distal electrode) is determined (e.g., recorded) at several different temporal locations as the electrode array is advanced into the cavity (here, a scala of the cochlea). By obtaining a record of the tip electrode at the 4 insertion points shown (by way of example), a contour of the modiolus wall 843 is developed, here, virtually/by way of a dataset, as shown by the dashed line 860 in FIG. 8L. Embodiments utilize these datapoints to make a reasonable estimate with as few as 3 or 4 points which is efficient/quick. In an exemplary embodiment, curve fitting techniques, such as computer implemented curve fitting techniques such as those utilized by CricketGraph™ or Excell™ or a computer based cubic spline algorithm, etc., can be utilized with these three or four points to estimate the contours of the modiolus wall and thus the location of the modiolus wall relative to some fixed reference (for example, the lateral wall 841, which lateral wall can be imaged, in at least some scenarios, with one or more of the imaging techniques detailed herein and/or can be located utilizing one or more of the electronic techniques detailed herein, and, for example, the entrance point into the cochlea for the array, such as a cochlear ostomy or the round window or the oval window, or more accurately, the bony structure around such/establishing the support for the membrane of those windows).

Note further that embodiments can utilize potentially, as few as two or even one point. For example, in an embodiment, there is historical data or atlas data taken from a number of humans relating to the actual contours of the lateral wall and/or the modiolus wall, such as by dissection techniques or other techniques (invasive imaging, etc.). Embodiments can include utilizing as few as one or two of the data points in combination with this data to construct a model of the modiolus wall. For example, demographic data and/or pre-operative (or during operative) imaging can be utilized to identify a class or a type of human cochlea to which the cochlea associated with the individual undergoing the implantation most closely corresponds or otherwise corresponds within a statistically acceptable value. The class of the type of human cochlea that is identified has pre-existing data associated therewith, such as by way of example only, contours and/or dimensions and/or other surface features, such as roughness for example, pitch, local tangent line with angular distance and/or depth, etc., of the cochlea in general, and the modiolus wall in particular. The two or one data points can be utilized in algorithm that also includes this pre-existing data to automatically develop a model of the modiolus wall based on the combined data. For example, the one or two data points can be virtually superimposed onto contours of a pre-existing modiolus wall model (or the points can be used to “virtually align” the pre-existing modiolus wall model) and correlating the locations of the points that are known (e.g., due to the depth of insertion and/or the angle of insertion, etc.) to the pre-existing modiolus wall module, the location of the modiolus wall of the individual can be estimated. And note that embodiments can utilize this pre-existing model concept with more than two or three or four data points.

Note further that any disclosure herein of insertion distance corresponds to an alternate disclosure of insertion angle and vice versa, unless otherwise noted, providing that the art enables such with respect to the overall teachings associated therewith.

In at least some embodiments, there are less than (i.e., 1), greater than and/or equal to 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 70, 80, 90, or 100 or more, or any value or range of values therebetween in one increment discrete data points associated with the tip portion (e.g., 43, 54, 19 to 59, etc.) that are obtained and/or utilized to construct the model or otherwise the data set that represents the modiolus wall of the individual.

Embodiments also include an electrode array that has a radiopaque component added to the traditional electrode array. By way of example only, FIG. 8M shows an electrode array 1466 that includes a radiopaque marker 838 in the tip portion of the array. The marker 838 is located forward of all of the electrode/more distal than all of the electrodes although in other embodiments, this could be different. The marker shown is a triangular marker that has a flat section facing the portion of the electrode array that is to face the modiolus wall when the array is inserted in the cochlea. The idea is that this gives the greatest reference and otherwise an identifiable reference plane for measurements, including computer-assisted measurements which can rely on tangent planes of surfaces for example (measuring the distance between such planes). Other types of markers can be utilized. In an embodiment, the marker is made of a material detailed above. In an exemplary embodiment, the marker's detailed herein can be markers that give off some form of radiation.

In an exemplary embodiment, the above-noted curve fitting techniques can be utilized to establish the trajectory 860. In an exemplary embodiment, the trajectory 860 can be utilized as a proxy for the modiolus wall 843. In an exemplary embodiment, a known offset can be applied. For example, based on demographic data or other data obtained associated with the cochlea of the individual, or utilizing a stock standard value, the location of the modiolus wall 843 can be estimated to be a certain value “inside” the trajectory 860. This owing to the fact that the imaging and/or the electrical data that provides the locations of the tip portion to obtain the data points results in a centroid that is away from the actual modiolus wall. Accordingly, the modiolus wall model/dataset can be constructed by obtaining local tangent lines of the trajectory 860 and moving those local tangent lines a distance that is normal to the tangent line towards the center of the spiral (away from the lateral wall 841), which distance could be less than, greater than, and/or equal to 0.01, 0.02, 0.03, 0.04, 0.05, 0.06, 0.07, 0.08, 0.09, 0.1, 0.125, 0.15, 0.175, 0.2, 0.225, 0.25, 0.275, 0.3, 0.325, 0.35, 0.375, 0.4, 0.45, 0.5, 0.55, 0.6, 0.65, 0.7, 0.8, 0.9, or 1 mm or more, or any value or range of values therebetween in 0.005 mm increments. Note further that the distance could be standard over the entire trajectory, or could be a distance that varies over the trajectory so as to take into account the fact that there is dimensional compression the further that the array is extended into the cochlea. For example, a reduction in the offset of five or 10 percent can be present for every 7½° of insertion angle by way of example only and not by way of limitation, or the change in offset can be staggered for every insertion angle and/or every distance of insertion, etc.

The point is that in an exemplary embodiment, a location of a portion of the electrode array is utilized as a proxy for the location of the modiolus wall and/or lateral wall. The location of the portion of the electrode array is a latent variable that indicates the location of the modiolus wall. In an exemplary embodiment, the portion of the electrode array will never be “inside” the curvature of the modiolus wall, unless there is a puncture, and such a scenario would result in a trajectory that is sufficiently aberrant as a matter of statistical and/or empirical or otherwise possible geometries of the cochlea that the algorithm would detect such. And in this regard, embodiments include algorithms that evaluate the tangent of the trajectory 860 over a range of locations along the trajectory and if there is a steep change or otherwise a change in the slope of the tangents within a given distance that is above a certain value (or below a certain value—e.g., if there is no change, such would indicate that the electrode array is going straight and thus is pierced tissue instead of following the contour of the cochlea), the algorithm indicates such and thus flags the data is not being representative of the modiolus wall for example and also can, in some embodiments, providing indication to the surgeon or otherwise a healthcare professional that there is a problem with the insertion, such as that there is a piercing of tissue by the array. Another exemplary event can be the insertion of the electrode array into a hypotympanic air cell. The array can be curled too tightly. Another example is coiling the array into the vestibule. These placements are unusual and can be detected by the teachings herein (such as for example, unusual impedances between multiple electrodes).

Still, in this exemplary embodiment (the embodiment shown in FIGS. 8H to 8L), the electrode array is taking the optimal path around the modiolus, and thus maintaining proximity to the modiolus throughout the insertion as is desired and otherwise is the goal of the insertion process.

The position of the tip portion relative to the visible lateral wall structures can be estimated by any technique that can identify the distance of the tip from the lateral wall as it is inserted. For example, the tip portion position relative to the lateral wall could be estimated by a series of x-rays, or a continuous or discontinuous fluoroscopy, or CT scans, or a number of electrical measurement techniques for identifying the position of the tip portion. For each location of the tip portion that is used as a datapoint, the insertion depth can be estimated by monitoring the length of the electrode array that has entered the cochlea. One way to do this is to take a visual record of which electrode contact is at the insertion point at each stage. This could be done by a manual visual check, or an electronic video system for example. Smart-Nav, which is available from Cochlear LTD, under its Surgical Care “product’ line, which can provide wireless, real-time actionable insights that support navigation during cochlear implantation surgery, could be used. In an embodiment, this system or another comparable system can use a measure of impedances as electrodes enter the cochlea to estimate insertion depth. Other ways to monitor insertion depth can also be applied including monitoring the extension of a robotic insertion system or a surgical navigation system. It is also possible to estimate insertion depth of an electrode contact using a Trans Impedance Matrix as described by Aebischer, P., Meyer, S., Caversaccio, M., & Wimmer, W. (2020). Intraoperative Impedance-Based Estimation of Cochlear Implant Electrode Array Insertion Depth. IEEE transactions on biomedical engineering, 68(2), 545-555. All of these techniques can be used in some embodiments.

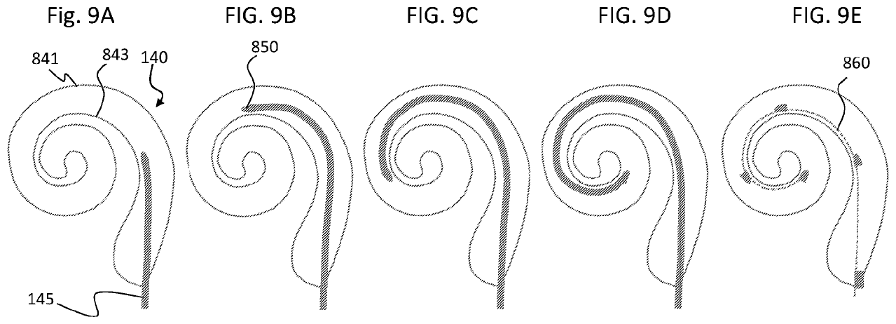

The trajectory of the entire electrode array in FIGS. 8H-8L follows the modiolus wall and finishes in a perimodiolar position, as desired. It is tempting to think that the location of the modiolus can therefore be detected simply by taking the location of all the electrode contacts in the final position. While this can happen, it is not always so. It can be that portions away from the tip portion, such as a middle portion of an electrode array for example, end up at a point somewhat distant from the modiolus wall as shown in FIGS. 9A to 9E. This can be due to over insertion or because the tip portion is hung up or otherwise experiences some friction forces against the modiolus wall, or due to over-insertion. It can be that the middle portion of an electrode array or these other portions follow a path that is somewhat distant from the modiolus during insertion. However due to the mechanics of a pre-curved electrode, the tip portion and/or the tip contact can still follow the modiolus in the same path as for the scenario in FIGS. 8H-8L, even if the rest of the electrode array is wide of the modiolus. This can happen due to the modiolus wall being somewhat rough or bumpy which creates friction of the tip portion against the modiolus wall and thus requires a slightly higher insertion force which causes the middle portion of the array to flex away from the modiolus. Therefore, it is still possible to record the position of the tip at a number of points during insertion allowing the contour of the modiolus wall to be estimated in the same way as for the scenario in FIGS. 8H-8L. This is shown in FIGS. 9A-9E. Here, in this example, the tip portion follows a similar path to that of FIGS. 8H-8L, however the mid-section of the array does not follow the modiolus, and the final position of the electrode array is not close to the modiolus. Despite this, the tip portion still tracks sufficiently close to the modiolus wall. By obtaining a record of the electrode array tip portion at the 4 insertion points shown (or however many points), a contour of the modiolus wall can be estimated from the trajectory 860, just as was done in the above scenario for FIGS. 8H-8L.

In an embodiment, the locations of other portions other than the tip portion are tracked/recorded. For example, embodiments track the location of one or more electrode contacts or even all the electrode contacts other than and/or including the tip portion (whether the tip portion includes one or more electrode contacts.) In an embodiment, the tracked portions are tracked relative to the lateral wall of the cochlea, which appears on imaging and/or electronic locational techniques. Again, the tracked portions are tracked relative to/while measuring insertion depth of the contact(s) being tracked.

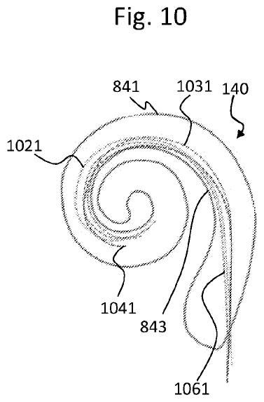

In this regard, FIGS. 8H-8L presented a single trajectory of the tip portion (e.g., the distalmost electrode contact). But by following/tracking or otherwise recording/determining positions of one or more other or all electrode contacts, a series of tracks/trajectories can be developed/estimated, and from one or more or all of these, a contour of the modiolus wall can be estimated. This concept can be conceptually seen in FIG. 10. FIG. 10 shows estimated insertion paths of 4 different electrode contacts (curves 1061, 1021, 1031, and 1041), which in this embodiment correspond to the four distalmost electrodes). Some of the electrodes take a path wide of the modiolus at different times. Different electrodes are closest to the modiolus at different points around the modiolus. Thus, by tracing out the electrode contact furthest away from the lateral wall (as measured for example in a direction normal to a tangent line of the lateral wall, with the direction normal being where the tangent line contacts the lateral wall) and/or closest to the center of the spiral and/or the local trajectory portions that have the smallest radius of curvature (inside) and/or the closest to the estimated modiolus at various depths/angles of insertion (the more the more accuracy in at least some embodiments), a more accurate estimate of the contour of the modiolus wall can be made. In some embodiments, this can create an estimate with more confidence than that which results from the single portion. This can be because there can be more data to draw on in estimating the contour of the modiolus wall. Corollary to this is that this can be utilized to eliminate or otherwise discount extraneous data points. By way of example, it should not be that at angular insertion 95° for example, one electrode is “inboard” (on the opposite side of the modiolus wall from the array), providing that there is no piercing (of the basilar membrane for example, or even the modiolus)—it is possible for an array to pierce the basilar membrane and trace around the scala vestibuli, and thus result in an incorrect estimation of the modiolus of the scala tympani—and thus any data that results from such a scenario that would not exist in a normal implantation can be discounted (or used to indicate that there is a problem with the implantation). Thus, the data point that is most inboard or otherwise the data point associated with the trajectory that has the smallest radius of curvature should be most indicative of the location of the modiolus wall. The electrode contact at issue should not be able to get closer to the center of the spiral than the modiolus wall.

Embodiments can thus include taking the various data points for respective insertion depths and taking the one that gives the “closest” value to the center spiral or the largest distance from the lateral wall (utilizing the above-noted constraints for example, utilizing the direction normal to the tangential surface) or the smallest radius of curvature, etc., and utilizing such to establish the estimate for the modiolus wall at that insertion depth. In an exemplary embodiment, all the trajectories can be averaged. A mean, median, and/or mode average can be used with respect to any of the just-noted values.

In an embodiment, discrete data points are obtained and correlated to insertion depth. For example, depths measured from the beginning, midpoint, and/or endpoint of an electrode contact when such point first enters the passage into the cochlea and/or first enters the duct of the cochlea can be used from which to measure “forward”/apical locations. For example, if electrode contact 1 is the most distal electrode, the distance could be measured in terms of electrode 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (in increasing numerology from the most distal electrode) meets the just-noted criteria. Thus, for example, the spatial datapoints associated with any one or more of the just-noted contacts can be obtained when the 10^{th}, 11^{th}, 12^{th}, 13^{th}, 14^{th} and 15^{th} electrode contact meet the just-noted criteria (e.g., the beginning of the contact enters the passage into the cochlea). Thus, 6 datapoints for electrode contacts 1, 2, 3, 4, 5, 6, 7, 8, and maybe 9 can be obtained, each correlated to the insertion depth. Note also, that actual distances can be used. These distances can be measured along the longitudinal direction of the array—if the most distal electrode contact is 25 mm away from the most proximal contact (as measured from the most distal locations of the electrode contacts, the most apical locations or the geometric centers), and each electrode is spaced apart by 1 mm (as measured from the geometric center) other than the most proximal electrode, there could be datapoints for electrode contact 1 for every 1 mm of advancement, or every ½ mm of advancement, etc. Indeed, if the imaging is constant or effectively constant, there could be a quasi infinite number of datapoints (if the data is analogue—if the data is digital, there will be a relatively high number).

Datapoints can be taken when the beginning, middle, and end of each contact enters the passage and/or as that portion enters the duct of the cochlea. This would of course provide more data points than if only the beginning, middle, or end were utilized.

In an embodiment, discrete datapoints are obtained and correlated to insertion angle for given portions of the electrode array.

Time could be used as a measurement regime, if the array is being inserted at a constant speed, or even a variable speed, if a computer system logs the speeds and accounts for the variations in speed. But even without some form of accounting for speed, or without using any measured timing that is directly correlated to a portion of the array (other than for example, initiation of the process—more on this in a moment), some exemplary embodiments do not include the measurement of distance or otherwise have a recordation of distance of insertion into the cochlea or angular insertion into the cochlea or timing of insertion. Embodiments that are based on imaging can have multiple datapoints by taking/obtaining multiple images separated by time, whether constant or non-constant separation timing. For example, consider a scenario of implantation where the cochlea and the portion of the array that is located in the cochlea is imaged every half second for example, or every second, or randomly within a given second period (so potentially, there could be two images separated by just under two seconds). Providing that there are images that show the portion of the array of interest, or at least images that enable identification of such portion relative to other portions of the array (although even then, in some embodiments, it can be sufficient to simply identify different portions at different locations within the cochlea—for example, electrode number 1 at 13 mm insertion depth into the cochlea, electrode number 3 at 15 mm insertion depth into the cochlea, electrode number 4 at 10 mm insertion depth into the cochlea, electrode number 12 at 7 mm insertion depth into the cochlea—if each one represents a spatial data point that can be utilized to estimate the location of the modiolus wall (e.g., because none of those electrode contacts can be inboard of the wall), such could be sufficient), such could be utilized to determine the location or otherwise estimate the location of the modiolus wall according to the teachings above. There can be utilitarian value with respect to being able to match the location of the modiolus wall relative to another anatomical portion of the cochlea, such as the lateral wall, and in this regard, the images will likely provide such reference, because the lateral wall, unlike the modiolus wall, is more readily imaged by the imaging techniques detailed herein. Still further, the opening into the cochlea would be something that would show up on imaging, and thus the modiolus wall estimate could be correlated thereto instead of or in addition to correlation to the lateral wall. In some embodiments, it is the portions of the array to which the various anatomical structures are correlated. Thus, with respect to any one or more of the contacts, the lateral wall in reality and/or the estimated modiolus wall could be correlated thereto.

In an exemplary embodiment, the various trajectories of FIG. 10 can be combined, such as by averaging, or by weighting (e.g., the trajectory established by the most distal electrode could be weighted more than say electrode 8, or another electrode, etc.) The combined (averaged) curve could then be used to estimate the modiolus wall.

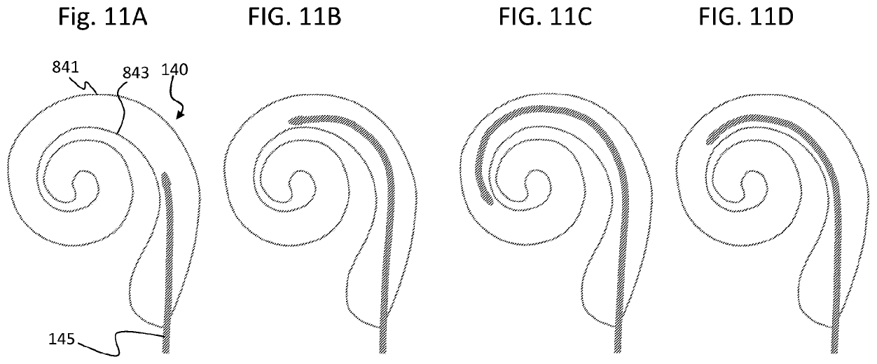

Embodiments include withdrawing the electrode array a bit/pulling the array backwards and also tracking the path of the portions of the array during and/or as a result of (at the completion of) this procedure. This is sometimes referred to herein as a partial withdraw procedure. In at least some scenarios, as will be described in greater detail below, this would tend to pull the electrode array that has bowed away from the modiolus wall back on to the modiolus. FIGS. 11A-11D show an exemplary scenario of such a procedure, where FIG. 11C presents the array 145 part-way through the full insertion process. If pulled back a short way, the electrode array will tend to pull back toward the modiolus as shown in FIG. 11D. A record of the location of one or more or all of the electrode contacts can be taken, such as relative to the lateral wall, or relative to a particular electrode contact, or to the opening, can be obtained. This adds to the total data and accuracy of the estimate of the contour of the modiolus wall. To be clear, such a pull-back technique could be done after an electrode array has been fully inserted, or potentially at one or more points part-way during insertion (e.g., less than, greater than and/or equal to 30, 35, 40, 45, 50, 55, 60, 65, 70, 75, 80, 85, 90, 95, or 100% or any value or range of values therebetween in 1% increments of the total distance inserted (from the most distal portion of the tip portion for example, or the furthest that the most distal electrode is inserted (leading portion, trailing portion or middle portion)) that will be the case at the point where the array is fully seated and where the array will be located at the completion of the implantation procedure/when “packing” of the opening (tissue placement to close the opening around the array) takes place. This technique could add further data and thus provide an improved estimate of the contour of the modiolus wall. Again, the measure could use the tip portion, the tip electrode contact or two or more or all contacts to gather more data, or any component of the array that can enable the teachings herein.

After recording the tip portion positions, or whatever portion positions are being used, a variety of smoothing and error checking algorithms could be used to weed out spurious results. For example, in a normal anatomy of a patent's (the individual's) cochlea, as determined by analysis of CT and/or MRI scans by way of example, preoperative or during operation, or by other analysis techniques (electrical for example), the contour of the modiolus wall would not likely include significant and sudden steps laterally or medially. Not that those techniques just mentioned might not definitively image (“see”) the modiolus wall, but those techniques could image other portions of the cochlea, or otherwise obtain data on other portions of the cochlea, which can be utilized as latent variables or otherwise as a proxy to estimate or otherwise gauge the likelihood that the modiolus wall is “normal” or abnormal. This can be based on a data set of previously collected data for other humans where the features of the modiolus wall are known or otherwise recognized for those other humans or otherwise can be based on a dataset for other humans where the features of other portions of the cochlea are known, whereby educated guess implementation it is extrapolated that the normal lateral wall for example indicates the presence of a normal modiolus wall etc. Thus, a recording that is inconsistent with the greater body of data would be regarded as spurious and not used in estimating the contour of the modiolus wall. Moreover, as noted above, if there are values that appear to be statistically aberrant from a given contour, those can be eliminated from the data set or otherwise from the data that is used to develop the overall contour.

Another aspect that can be used in error-checking is determining whether the electrode array has dislocated from the scala tympani into the scala vestibuli, or whether the array has been inserted directly into the scala vestibuli, or dislocated from scala vestibuli into the scala tympani. If any of these conditions have occurred, the estimation of the contour made by the method described herein would be deemed not to be valid in at least some instances, or otherwise suspect. These conditions can be detected and reported to an algorithm that executes the teachings herein, as will be described below, which could report a potential anomaly in the analysis for this reason. Translocations from ST to SV and vice versa can be detected by analysis of psychophysical measures as described for example by Mittman et al 2015 (Intraoperative Electrophysiologic Variations Caused by the Scalar Position of Cochlear Implant Electrodes, 36:1010-1014, Otology & Neurotology 2015). Alternatively, intraoperative imaging can be used to determine in which scala an electrode is placed as described by Mitchell, et al, Cost-Effectiveness of Intraoperative CT, Scanning in Cochlear Implantation in Fee-for-Service and Bundled Payment Models, Ear, Nose & Throat Journal, 2020. Embodiments include implementing one or more of those methods/using devices implementing one or more of those methods/systems implementing one or more of those methods, to determine if any one or more of the just detailed deleterious scenarios has occurred, and discounting and/or flagging or otherwise throwing out the data if such has occurred.

Embodiments include approximating the location of the modiolus wall because techniques utilized to identify the position of an electrode contact or other reference component used (probe or marker, etc.) relative to the lateral wall are subject to the precision of the measurement technique. This has implications with respect to embodiments that utilize the lateral wall as the frame of reference to place the estimated location of the modiolus wall. Thus, there can be error upon an error. Even with the utilization of cone beam CT, there is a level of error with respect to locating the modiolus wall. Thus, embodiments can include error accounting features that provide a tolerance band with respect to the ultimate determination as to how far the electrode array is from the modiolus wall or otherwise whether or not a placement of the electrode array is acceptable. For example, it could be that the distances from electrodes 8, 9, and 11 are outside the acceptable tolerance band, but a level of confidence of the location of the modiolus wall is sufficiently low enough that it may not warrant adjustments of the location of the electrode array, at least if the electrode 10, which is in between the electrode 8 and electrode 11, is found to have an acceptable value. The point is that the system can include a level of confidence evaluation feature that can account for the fact that the values with respect to the locations of the distances are not precise but are instead estimates, even if they are keyed off of the location of the lateral wall, and thus can at least advise the healthcare professional accordingly that the data with respect to the distances are locations is subject to a higher level of error than that which would otherwise might be the case or otherwise is normal. And the converse can be true, the system can indicate that there is a high level of confidence such that even if there is only one value that is outside of the tolerance range, an indication can be made that the electrode array is not close enough to the modiolus wall.

Note also that embodiments include a system that automatically develops a separate estimate of the location of the modiolus wall based solely on the location of the lateral wall. In this regard, at least some exemplary embodiments are practiced where the modiolus wall follows the lateral wall with a relatively well-known deviation therefrom with respect to historical data associated with other patients and other people, especially those that are of similar demographics or have other similar characteristics to the patient in question. In this regard, without the use of the markers herein, a preoperative image or a during operation image can reveal the contours of the lateral wall, and from those contours of the lateral wall, the contours and/or location of the modiolus wall can be estimated. The techniques herein can utilize that initial estimate to further refine the estimate of the location of the modiolus wall, such as by utilizing the locations of the markers as detailed above. And if there is a large deviance between the initial estimate and the developed estimate utilizing the markers, it could be that the developed estimate or all estimates are ignored, or the initial estimate is ignored or otherwise discounted to some extent or the latter is discounted. Corollary to this is that a second set of imaging could be taken or otherwise obtained to obtain a new lateral wall—modiolus wall relationship estimate, and if this new relationship is closer to the estimated contours of the modiolus wall developed via the marker process, this latter image can be utilized and the former image could be discounted or otherwise eliminated, or more specifically, the initial estimate of the modiolus wall developed with the initial set of imaging's could be discounted or otherwise eliminated. Alternatively, and/or in addition to this, the medical device can be completely withdrawn and the data set could be at least temporarily shelved and a new set of data can be obtained with a new insertion process of the medical device into the cochlea, and if the data with the second insertion process better corresponds to the estimated locations of the modiolus wall based on the estimated location of the lateral wall, this later data set can be utilized instead of the former data set. Also, the teachings herein can have utilitarian value with respect to a revision surgery. For example, in a revision surgery, the modiolus could be mapped as the electrode array is withdrawn from the cochlea. This assumes that the device is still basically working and not a complete failure. By way of example, this could be the case in the following instances:
Removal for some other surgery incompatible with keeping the device in place.
Removal due to infection.
Removal due to some number of electrodes non-functional (but still enough working to do an analysis).
Removal due to question on the performance due to unknown causes.
   In an embodiment, an algorithm would disregard, for example, any electrodes that are open or short circuit which can happen at any time. The point is that the teachings herein can be used also during a withdrawal process. Embodiments directed to withdrawal during an insertion process can be used during a revision surgery, and any disclosure herein of the former corresponds to doing so during a revision surgery. Embodiments include taking any teaching herein and reversing the action (withdrawal instead of advancement) providing that the art enables such.

Note further that electrical techniques can be utilized to determine the location or the contours of the lateral wall. This can be done in at least some exemplary embodiments where the electrodes are band electrodes that extend all the way around the electrode array or there are electrodes that are located on the lateral side of the electrode array, consistent with the teachings of U.S. provisional patent application No. 63/277,253 and PCT/IB2022/060473, where the teaching thereof can be utilized herein to establish the locations of the lateral wall and/or the modiolus wall, at least for error control or otherwise locational estimate refinement.

Still, embodiments include developing a model of the modiolus wall without reference to the features of the lateral wall.

In an embodiment, the final position of the electrode array in the scala relative to the lateral wall, which can be better imaged and otherwise the location thereof can be better estimated or otherwise determined, can be compared to the various paths identified herein to confirm that the final position of the electrode array throughout its length or a portion of its length, such as any of the percentages detailed herein, to determine or estimate how close the electrode array is to the modiolus wall, or otherwise to determine a state of closeness, such as optimally close or otherwise clinically close. By way of example only and not by way of limitation, if the final position of the electrode array as it appears in the imaging takes a tract that is aberrant from the contours of the lateral wall, a determination can be made that it is likely that the electrode array is not optimally positioned relative the modiolus wall. That said, if the tract is not aberrant, that in and of itself does not indicate that the electrode array is clinically close to the modiolus wall. Put another way, the features associated with the lateral wall could be utilized to identify false positives in at least some embodiments. These features may not, at least in some embodiments, be able to identify false negatives.

In any event, consistent with the teachings herein, embodiments can utilize the estimated location of the model of the data set associated with the estimated modiolus wall and compare that estimated modiolus wall location to the locations of one or more components on the electrode array, such as any of the electrodes detailed herein, to evaluate how well the electrode array is positioned relative to the modiolus wall, where the closer to the wall the better. That said, it could be that there is utilitarian value in simply determining that the relative locations are relatively constant. That is, it could be that there is a distance from the electrode array to the modiolus wall that is larger than that which would be desired, but if that distance is constant or otherwise falls within a narrow band, that could be acceptable because it is sufficiently uniform. That said, in at least some exemplary embodiments, the goal is to get the context of the electrode array as close as possible to the modiolus wall. Because the location of the modiolus wall is often difficult to determine, the teachings detailed herein allow for an evaluation as to the closeness of the electrode array to the modiolus wall, or more specifically, the context of the electrode array relative to the modiolus wall, or whatever marker is being utilized, having an accuracy that hereto for did not exist, at least not in a surgical setting. And to be clear, in at least some exemplary embodiments, at least one or more or all of the method actions detailed herein are executed within 1, 2, 4, 5, 6, 7, 8, 9, 10, 15, 20, 25, 30, 35, 40, 50, 60, 70, 90, or 120, or any value or range of values therebetween in one increment minutes of the first entrance into the cavity (e.g., a drill opening the passageway) or the first entrance of the medical device at issue into the cavity and/or the completion of the surgery, where, for example, the closure procedure begins.

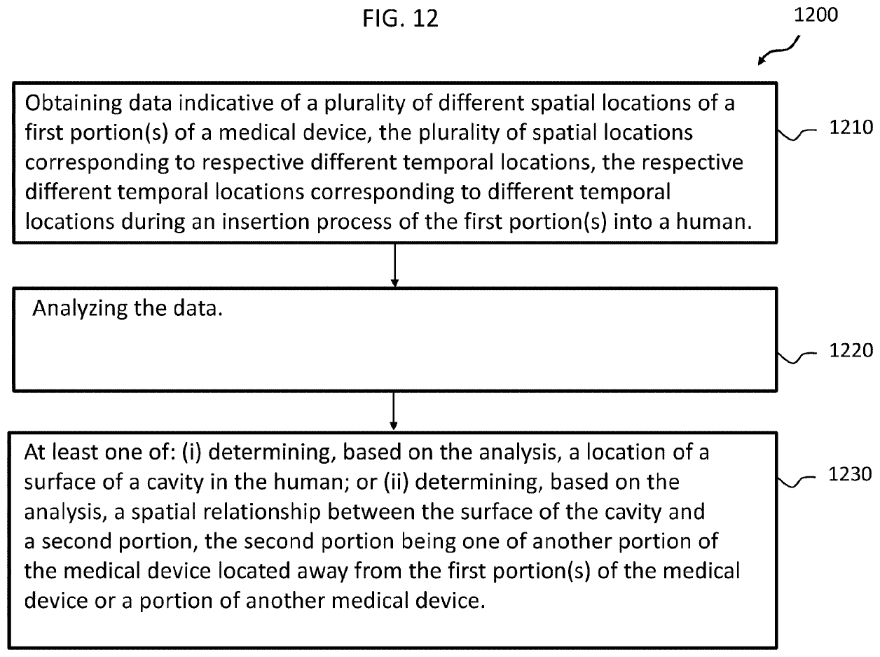

In view of the above, there are methods, such as the method represented by the flowchart of FIG. 12, method 1200. Method 1200 includes method 1210, which includes obtaining data indicative of a plurality of different spatial locations of a first portion(s) of a medical device (as noted above, different portions can be used to develop different trajectories, and the trajectories can be combined into a single trajectory or used individually to develop a dataset—thus, here, there could be one first portion (e.g., electrode 1, and there would be two or more different spatial locations for electrode 1) or there could be two first portions (e.g., electrode 1 and 2, and there could be a spatial location for electrode 1 and a different spatial location for electrode 2 (not the same spatial location as electrode 1), the plurality of spatial locations corresponding to respective different temporal locations, the respective different temporal locations corresponding to different temporal locations during an insertion process of the first portion into a human. In an embodiment, this medical device can correspond to an electrode array, such as a cochlear implant electrode array detailed above. The first portion of the medical device can correspond to the tip portion for example or any one or more of the electrode contacts noted above. The tip portion can include the most distal electrode as noted above.

The above notes that the spatial locations are obtained at different temporal locations. In an embodiment, there can be utilitarian value for doing so, especially if such is associated with respect to a single portion, such as the first electrode. The elapsed time is needed to move that first electrode from one position to another position and then another position to establish the data points as noted above. That said, in embodiments where different portions of the electrode array are being utilized, such as different electrode contacts, it is possible that the different spatial locations could be obtained at a given instant in time. By way of example only and not by way of limitation, in an exemplary embodiment, if the electrode array has been pulled back for example in the partial withdrawal procedure noted above, this could have the effect of moving a plurality of electrodes closer to the modiolus wall than that which would otherwise be the case, which could be closer than that which would be achieved during the movement of the electrode array into the cochlea or otherwise forward. Thus, in this exemplary scenario, it could be that the electrode arrays pulled back slightly or however much as utilitarian, thus “snugging” the array against the modiolus wall, and then a single images taken, and the plurality of different spatial locations is obtained from the image, where the plurality of different spatial locations are four different electrode contact by way of example.

Method 1200 further includes method action 1220, which includes analyzing the data. In an exemplary embodiment, the action of analyzing the data can correspond to the establishment of the trajectory or otherwise ascertaining the trajectory noted above. Method 1200 further includes method action 1230, which includes at least one of (i) determining, based on the analysis, a location of a surface of a cavity in the human or (ii) determining, based on the analysis, a spatial relationship between the surface of the cavity and a second portion, the second portion being one of another portion of the medical device located away from the first portion of the medical device or a portion of another medical device. With respect to the first option of method action 1230, this can correspond to determining the location of the modiolus wall as noted above, where the cavity is the cochlea of the human. In an exemplary embodiment, this can correspond to utilizing the trajectory that was developed as a result of the analysis of method action 1220 to determine the location of the surface, here, the modiolus wall. With respect to the second option, the location of the modiolus wall need not necessarily be developed per se or otherwise determined. Instead, there is a determination of a spatial relationship between the surface of the cavity and a second portion of the medical device. Here, the second portion could be an electrode contact away from the first electrode contact that was the subject of method action 1210 with respect to the first portion of the medical device. This can be any one or more of the electrode contacts of the electrode array providing that the electrode contact(s) do not correspond to that which is part of the first portion.

Note also that the second option in method action 1230 refers to the possibility that the second portion can be a portion of another medical device. Here, in an exemplary embodiment the medical device of method action 1210 can be the above-noted probe, and this probe is utilized to obtain the data that is analyzed in method action 1220. Consistent with the teachings above, this probe could be withdrawn from the cochlea, and then the cochlear implant electrode array inserted, into the cochlea, where, during the insertion process, by way of example, imaging techniques are utilized to determine the location of one or more of the electrodes for example relative to the surface of the cavity. Here, the lateral wall can be utilized as a reference that enables the spatial relationship between the surface of the cavity, here, the modiolus wall, and the portion of the electrode array to be determined.

In an embodiment of method action 1230, where the method includes determining a location of a surface of the cavity in the human, that surface can be the modiolus wall as noted above. This location can be a location that is relative to another location of the cavity, such as the lateral wall as noted above or the entrance into the cavity, such as the cochleostomy, etc. In an exemplary embodiment, there can be an additional method action of utilizing that determined location to evaluate the efficacy of the placement of the cochlear implant electrode array or otherwise determine if the electrode array is placed sufficiently close to the modiolus wall. In an exemplary embodiment, this can be performed by eyeballing for example an image of the array onto which is superimposed the estimated location of the modiolus wall, which location can be obtained from method action 1230. This can be performed automatically instead of eyeballing, such as utilizing distance determining algorithms which can be surface and the portions of the array at issue, such as, for example, the electrodes. Predetermined values such as go no/go values can be utilized to determine whether or not the placement is acceptable. As will be described in greater detail below, the system could automatically indicate whether or not the placement is acceptable or otherwise recommend that the array be moved and re-placed about the modiolus wall.

With respect to the second option in method action 1230, determining the spatial relationship between the surface of the cavity and the second portion, the location of the modiolus wall or otherwise the surface of the cavity need not necessarily be determined. Here, it is sufficient to simply obtain the spatial relationships between the surface and the second portion of the medical device or a portion of another medical device. For example, the distances between the surface and one or more electrodes can be determined. This information can be evaluated manually to determine whether or not the placement of the electrode array is acceptable and/or can be analyzed automatically utilizing an algorithm that can determine the distances and evaluate those distances and determine whether or not the placement of the array is acceptable or recommend moving and readjusting the location of the array.

In an embodiment, respective distance between one or more or all of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 electrodes and the surface (modiolus wall) can be determined, and based on the determined distance, an average (mean, median, and/or mode) can be determined and this compared to a go-no go value to determine whether or not the array should be repositioned. In an embodiment, a maximum or a minimum distance can be the qualifier. In an embodiment, a subset of values can be used (e.g., if say three electrodes have “bad” values, but all the rest have “good” values, that might be acceptable, but if four have “bad” values, then that might not be acceptable—channels of the cochlear implant could be adjusted to avoid 1 or more of the electrodes that have the “bad” values).

In an exemplary embodiment, there are less than, greater than, and/or equal to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 70, 80, 90, 100, 125, 150, 175, 200, 225, 250, 275, 300, 350, 400, 450, 500, 600, 700, 800, 900, 1000, 1250, 1500, 1750, 2000, 2500, 3000, 3500, 4000, 4500, 5000, 6000, 7000, or 8000, or any value or range of values therebetween in 1 increment spatial locations in method 1200.

In an embodiment, the method includes determining the location of the surface of the cavity in the human based on the analysis and the surface is a surface that is not readily imaged with a standard X-Ray or a standard CT scan or a cone beam CT scan or any one or more of the imaging techniques detailed herein. In an embodiment, a location of the surface cannot be determined, including accurately determined, within plus or minus 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 70, 80, 90, 100, 125, 150, 175, 200, 225, 250, 275, 300, 350, 400, 450, 500, 600, 700, 800, 900, 1000 micrometers, or any value or range of values therebetween in 1 micrometer increment of the actual location over more than 30, 35, 40, 45, 50, 55, 60, 65, 70, 75, 80, 85, or 80% of its surface area using the noted imaging techniques or those noted herein. By way of example only and not by way of limitation, in an exemplary embodiment, the surface is a modiolus wall of the cochlea, and the location thereof cannot be determined relative to the lateral wall of the cochlea within the above-noted ranges. For example, at a location on the lateral wall, in a direction normal to the tangent surface thereof, the modiolus wall cannot be placed a distance from the lateral wall within for example, ±800 μm.

In an exemplary embodiment, there are at least three different spatial locations that are obtained. Also, the first portion(s) can be a first portion (singular—e.g., a discrete component, such as electrode number 1, vs. multiple electrodes). In the exemplary embodiment, the spatial locations correspond to increasing depth insertion of the first portion. For example, this can correspond to increasing depth by less than, greater than and/or equal to 0.5, 1, 1.5, 2, 2.5, 3, 3.5, 4, 4.5, 5, 5.5, 6, 6.5, 7, 7.5, 8, 8.5, 9, 9.5, 10, 10.5, 11, 12, 13, 14 or 15 mm or any value or range of values therebetween in 0.1 mm increments. (Where these distances are along a curve when the array curves.) In an embodiment, with respect to angular insertion, this can correspond to increasing the angular insertion by less than, greater than and/or equal to 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 70, 80, 90, 100, 125, 150, 175, or 200 degrees, or any value or range of values therebetween in 1 degree increments. Thus, the difference between the spatial locations can be any of the just-noted values.

In an exemplary embodiment, the method includes determining the location of the surface of the cavity in the human based on the analysis, where the analysis can include using the at least three different spatial locations as a proxy for the surface. (Any of the numbers disclosed herein, and the values need not be the same. For example, 30 different spatial locations can be obtained, and only 20 of those spatial locations can be utilized as a proxy for the surface.) In an exemplary embodiment, the data indicative of a plurality of different spatial locations is based on imaging obtained during the insertion process. By “based on,” it is meant that the data can be obtained directly from the imaging, or can be obtained based on data based on the image (e.g., a touched up image or a wireframe model developed from the image, etc.).

In an embodiment, the first portion is a tip portion of a cochlear implant electrode array. As noted above, this can include the first electrode. This can include a beryllium bead or the like in the tip that enhances imaging.

In an embodiment, where the method includes determining the spatial relationship between the surface of the cavity and the second portion, where the second portion can be another portion of the medical device located away from the first portion of the medical device, wherein the second portion is an electrode of the array located less than (e.g., 1), greater than, and/or equal to 2, 3, 4, 5, 6, 7, 8, 9, 10, 11, 12, 13, 14, or 15, or any value or range of values therebetween in 1 increment electrodes away from the first portion. In an embodiment, the second portion is located less than, greater than and/or equal to 3, 3.5, 4, 4.5, 5, 5.5, 6, 6.5, 7, 7.5, 8, 8.5, 9, 9.5, 10, 10.5, 11, 12, 13, 14, or 15 mm, or any value or range of values therebetween in 0.1 mm increments away from the first portion (closest parts for example). In an embodiment, where the medical device is a cochlear implant electrode array, the first portion is distal of the most distal portion of electrode 1, 2, 3, 4, or 5, and the second portion is proximal the most proximal portion of electrode 1, 2, 3, 4, 5, 6, 7, 8, 9, 10, 11, 12, 13, 14, 15, 16, or 17 (and, in some embodiments, distal of the most distal portion of at least one of those electrodes, or any range therebetween/thereof).

In an embodiment, the first portion(s) can be any one or more of electrodes 1, 2, 3, 4, 5, 6, 7, 8, 9, 10, 11, 12, 13, 14, 15, 16, 17, 18, 19, 20 and the second portion can be any other of electrodes 1, 2, 3, 4, 5, 6, 7, 8, 9, 10, 11, 12, 13, 14, 15, 16, 17, 18, 19, 20, and the second portion can be more than one of the electrodes. There can be 1, 2, 3, 4, 5, 6, 7, 8, 9, 10, 11, 12 or more or any value or range of values therebetween of first portion(s), and there can be 1, 2, 3, 4, 5, 6, 7, 8, 9, 10, 11, 12 or more or any value or range of values therebetween of second portions.

Note also that if a second portion is the same as the first portion(s), that does not mean that the method 1200 is not met. If there is a first second portion that is different from the first portion(s) that is enough.

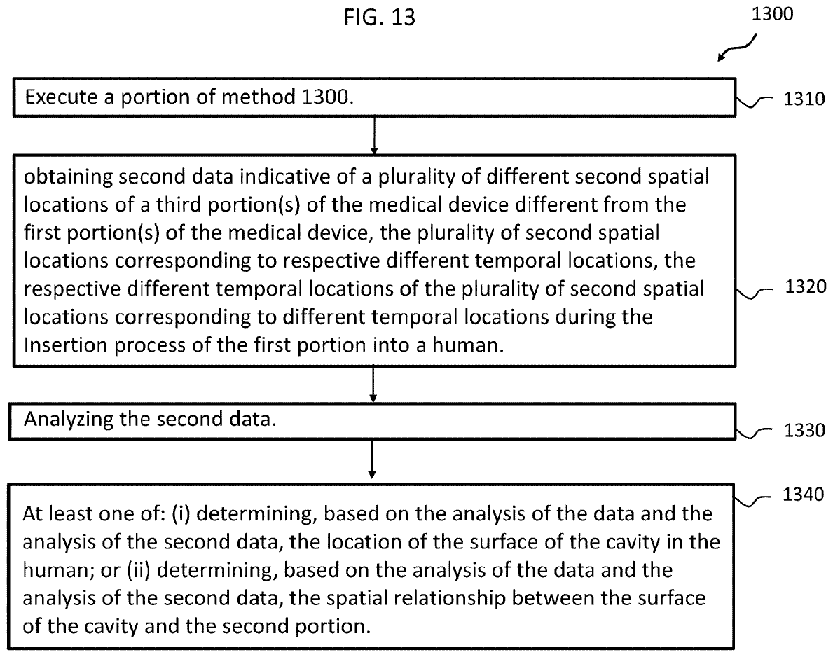

FIG. 13 shows another exemplary method, method 1300. Method 1300 includes method action 1310, which includes executing a portion of method 1200, such as method action 1210. Method 1300 includes method action 1320, which includes obtaining second data indicative of a plurality of different second spatial locations of a third portion(s) of the medical device different from the first portion(s) of the medical device, the plurality of second spatial locations corresponding to respective different temporal locations, the respective different temporal locations of the plurality of second spatial locations corresponding to different temporal locations during the insertion process of the first portion into a human. But as noted above, in some embodiments, as with the first portion(s), the temporal locations of the third portion(s) need not be different. Any number noted herein of the first portion(s) can correspond to those of the third portion(s), and they need not be the same (this is stated in the interest of textual economy). Method 1310 includes method action 1330, which includes analyzing the second data. Note that the action of analyzing the second data can be executed as part of method action 1220 as part of the same analysis. The analysis of method action 1330 can parallel the analysis of method action 1220.

Method 1300 includes method action 1340, which includes at least one of (i) determining, based on the analysis of the data and the analysis of the second data, the location of the surface of the cavity in the human or (ii) determining, based on the analysis of the data and the analysis of the second data, the spatial relationship between the surface of the cavity and the second portion. As with method action 1330 and method action 1220, this can be part of method action 1230/can parallel the analysis of method action 1230.

In an embodiment, any of the features noted above with respect to the first portions and/or the second portion can correspond to the third portions, providing that the limitations on duplication are addressed (stated in the interest of textual economy).

In an embodiment, there is a method that also includes the action of obtaining third data indicative of a plurality of different third spatial locations of a fourth portion(s) of the medical device different from the first portion(s) and third portion(s) of the medical device, the plurality of third spatial locations corresponding to respective different temporal locations, the respective different temporal locations of the plurality of third spatial locations corresponding to different temporal locations during the insertion process of the first portion(s) into a human. As with the second data, the fourth portion(s) can be any of those detailed above providing the duplication limitations are met. The method includes analyzing the third data, which can be part of the analysis of the other data. The method also includes at least one of (i) determining, based on the analysis of the data and the analysis of the second data and the analysis of the third data, the location of the surface of the cavity in the human or (ii) determining, based on the analysis of the data and the analysis of the second data and the analysis of the third data, the spatial relationship between the surface of the cavity and the second portion. And again, the temporal locations need not be different in some embodiments. Any of the second data shorthand descriptions above apply to the third data in an analogous matter.

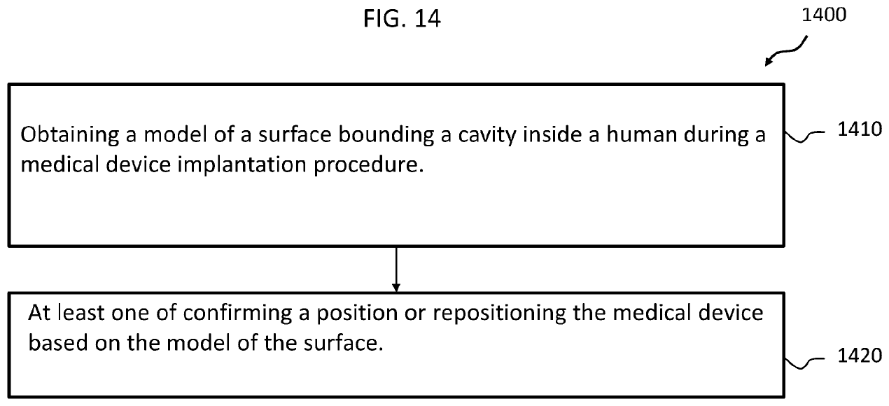

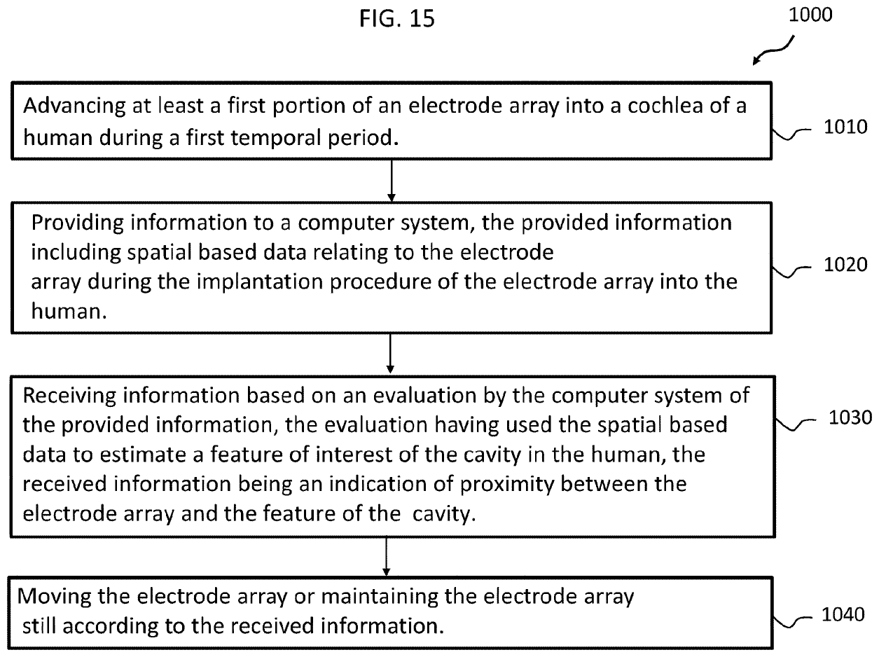

FIG. 14 presents another exemplary flowchart for an exemplary method, method 1400, which includes method action 1410, which includes obtaining a model of a surface bounding a cavity inside a human during a medical device implantation procedure. In an exemplary embodiment, this can be executed utilizing any one or more of the techniques detailed above. It is noted that the action of obtaining a model does not require that the actor develop the model. In an exemplary embodiment, during a cochlear implantation surgery, a healthcare professional could upload the spatial locational data associated with the portion(s) of the medical device to a remote server, which server automatically analyzes the data and develops the model of the surface, such as the model of the modiolus wall. The server would then provide the model to the healthcare professional. The healthcare professional's receipt of the model would be obtaining the model. That said, in an exemplary embodiment, the model could be developed on site at the healthcare facility where the operation is taking place, such as utilizing computer devices at the facility.

The obtained model could be a virtual model of the surface; a digital model. The model can be a 3D printed model. In an exemplary embodiment, the model could be displayed on a computer screen or in a virtual reality system. The model could be displayed in a vision field of the surgical microscope. The model can be presented as a hologram.

Method 1400 further includes method action 1420, which includes at least one of confirming a position or repositioning the medical device based on the model of the surface.

In an embodiment, the cavity is an aorta or a ventricle of a heart of the human. In an embodiment, the cavity is a cranial cavity of the human. In an embodiment, the cavity is a cavity in an eye of a human. In an embodiment, the cavity is a cochlea of the human. In an embodiment the cavity is a vestibule (such as where the array is used to treat balance problems).

In an exemplary embodiment, the model is based on data based on data having a correlation with tactile contact between the surface and the medical device. By data based on data, this can be the data that has the correlation, or can be a manipulated version or an extrapolated data from the data that has the correlation. As described above, in an exemplary embodiment, the tip portion of the medical device can be in direct contact with the surface during the insertion process that is part of the implantation procedure of the medical device. This does not necessarily mean that the marker of the tip portion is in contact with the surface. For example, if the marker that is utilized is electrode number 1, that electrode need not be in contact with the modiolus wall to achieve the tactile contact. It is sufficient that another portion of the tip portion be in contact with the wall. Note further that the marker need not necessarily be part of the tip portion in this exemplary embodiment. This embodiment simply requires that the model is based on data having a correlation with tactile contact between the surface and the medical device.

In an embodiment, the model is based on data based on data obtained while the medical device is in the cavity. Here, the data upon which the model is based need not have been obtained while the medical devices and the cavity. It is only required that the data upon which the model is based be based on data that was obtained while the medical device is in the cavity. In an exemplary embodiment, the model is based on data based on data obtained while the medical device is being inserted into the cavity. In an embodiment, the model of the surface that is developed is developed and/or obtained (recall that the action of obtaining can include the action of developing) within 30, 25, 20, 15, 10, 9, 8, 7, 6, 5, 4.5, 4, 3.5, 3, 2.5, 2, 1.5, 1, 0.5 minutes, or any value or range of values therebetween in 10 second increments from the first entrance of the medical device into the cavity and/or the establishment of an artificial opening from outside the cavity into the cavity and/or from the completion of the action of obtaining the data that is utilized to develop the model (e.g., the obtaining of the last spatial location).

Consistent with the teachings detailed above, the model is based on data based imaging of the medical device while the medical device is in the cavity and/or electrical measurement techniques while the medical device is in the cavity. In an exemplary embodiment, the imaging of the medical device is imaging of the medical device during the implantation procedure, such as while the medical device is being advanced into the cavity, and the electrical measurement techniques are techniques implemented during the implantation procedure, such as while the medical device is being advanced into the cavity. In an exemplary embodiment, the imaging and/or the electrical techniques are obtained/implemented (and the data associated therewith is created) while the medical device is moving, while in other embodiments, the imaging and/or the electrical techniques and the associated data is created within 60, 50, 40, 30, 20, 10, 5 or 1 second or any value or range of values therebetween in one second increments from a movement of the medical device (e.g., within 3 seconds of when the medical device was last moved).

It is briefly noted that while the phrase model is used herein, any such disclosure corresponds to an alternate disclosure of a data set and vice versa in the interest of textual economy, providing that the art enables such, unless otherwise noted. Thus, in an exemplary embodiment, method action 1410 can entail obtaining a data set of a surface bounding a cavity, and the action of confirming a position or repositioning the medical device is based on the data set of the surface (dataset representing the surface).

In an embodiment, method 1400 includes adjusting the medical device based on the model of the surface. As was described briefly above and will be described in greater detail below, the location of the medical device relative to the estimated location of the modiolus wall can be evaluated, and if the medical device is in a position that is not as utilitarian as that otherwise could be, a determination can be made that the medical device should be adjusted or more accurately, the position of the medical device should be adjusted. In an exemplary embodiment, a system could be configured to make this determination automatically, and convey a recommendation to the surgeon or other healthcare professional. Indeed, with respect to embodiments that are implemented utilizing a robotic system, the system could automatically reposition the medical device. In an exemplary embodiment, the surgeon confirm the position of the medical device based on the model of the surface. By way of example, an image of the surface could be presented on a computer screen with the cochlear implant electrode array superimposed over the surface. The computer system could have 3D abilities so that the surgeon or other healthcare professional could “rotate” or otherwise move the view to evaluate the relative positioning of the electrode array to the surface. This could be implemented using that said, this could be implemented utilizing the standard technologies a CAD system or the framework of such (e.g., Dassault's CATIA™). That said, this can be implemented utilizing technology such as that found on web-based product advertisements that enable a product to be rotated in three dimensions so that a view from any angle or very many angles can be obtained.

The system could augment or highlight distance. For example, a color coded system could be used where blue is “close” and red is “far.”

Embodiments have focused on the modiolus wall as being the surface. Embodiments could instead of or in addition to this include the lateral wall as the surface.

In view of the above, there is a method which includes the action of advancing at least a first portion of an electrode array into a cavity (e.g., a heart cavity or a cochlea) of a human (a recipient) during a first temporal period. In an embodiment, this is part of an implantation procedure (e.g., a cochlear implant implantation procedure) done in a surgery room or a health care facility. This can correspond to inserting an electrode array partially or completely into the cavity, such as a cochlea. (Embodiments will focus on the cochlea by way of example and not by way of limitation.) In an embodiment, of the total length of the array to be inserted and that will be inserted upon completion of implantation, at least and/or equal to and/or more than ABC %, where ABC 15, 20, 25, 30, 35, 40, 45, 50, 55, 60, 65, 70, 75, 80, 85, 90, 95, or 100 or any value or range of values therebetween in 1 increments, of the length of the array can be inserted into the cochlea by the point that any one or more of the method actions herein are executed.

In an embodiment, there is an action of advancing the array by at least and/or equal to and/or more than ABC % of the length of the array. For example, any one or more of the actions herein regarding obtaining spatial data can be executed by inserting less than 7% of the length of the array, which results in 67% of the array being inserted into the cochlea. Here we are keying the amount of insertion during an insertion action to the entire length of the array. An amount that is 7% of the length of the array is inserted. That could be the first 7% or the last 7%, or anywhere in between. In this regard, given temporal periods can be the variable that defines the action. In this regard, a first temporal period can be a period that is less than, equal to or greater than DEF seconds, where DEF is 0.1, 0.2, 0.3, 0.4, 0.5, 0.6, 0.7, 0.8, 0.9, 1.0. 1.1, 1.2, 1.3, 1.4, 1.5. 1.6, 1.7, 1.8, 1.9, 2, 2.25, 2.5, 2.75, 3, 3.25, 3.5, 3.75, 4, 4.25, 4.5, 4.75, 5, 6, 6.5, 7, 7.5, 8, 8.5, 9, 9.5, 10, 11, 12, 13, 14, 15, 16, 17, 18, 19, 20, 25, 30, 35, 40, 45, or 50, or any value or range of values in 0.025 increments. The reason for the fine increments can be for the discretization of the movement(s) and/or positions of the array. In this regard, a constant movement of the array can be broken down into discrete movements. Conversely, some scenarios of implantation can correspond to insertion of the array in a slow and/or methodical step-wise fashion, with some forward and backward movements and/or or pauses (intentional or unintentional). The teachings herein capture those movements, and convert them to numerical format, by, for example, receiving locational input using the devices disclosed herein and establishing a dataset of location vs. time and/or speed vs. time, and thus create a virtual “log” of the movements. This can be converted to an equation (curve fitting) or just be used as a discrete dataset. The time entries can be constant or can vary, providing that such has utilitarian value. Accordingly, there could be a dataset for the first temporal period where, for example, at 0.4 seconds, the insertion depth is 3 mm or X percent, at 0.6 seconds, the insertion depth is 3.4 mm or Y percent, etc. This is logged in some embodiments. And note that while the embodiments herein are often and typically described in terms of distance of insertion (e.g., 5 mm), embodiments can also be implemented where the amount of insertion is presented in terms of angular insertion amount. Any disclosure herein corresponding to a distance of insertion and/or withdrawal corresponds to an alternate disclosure of an angular amount of insertion and/or withdrawal amount. And embodiments include providing the instructions a recommendation in terms of angular amount as opposed to distance, etc.

But note that in some embodiments, there is no temporal and/or distance/angular correlation.

Embodiments include insertion actions where the electrode array is inserted over a time period such as, for example, from first entry of the array into the opening into the cochlea to final placement of the electrode array into the cochlea and/or to the first entrance of “packing” into the opening (packing tissue around the array to seal the opening), the time is less than and/or equal to 10, 9, 9, 7, 6, 5, 4, 3, 2, or 1 minutes, or any value or range of values therebetween in 0.1 minute increments (e.g., 7.3 minutes, 3.4 minutes, 2.1 to 5.5 minutes, etc.). In an embodiment, the array is inserted in a step-wise fashion (e.g., no more than 10, 9, 8, 7, 6, 5, 4, 3, 2, or 1 mm, or any value or range of values therebetween in 1 mm increments within a 30, 25, 20, 15, 10, or 5 second intervals, or any value or range of values therebetween in 1 second increments).

There can also be an action of providing information to a computer system, the provided information including spatial based data relating to the electrode array during the implantation procedure of the electrode array into the human. In an embodiment, this is done automatically. This can be the imaging and/or electrical data disclosed herein. The computer system can implement one or more of the method actions herein/has one or more of the functionalities detailed herein, and such can be executed automatically.

By way of example only and not by way of limitation, in an exemplary embodiment, there is a sensor on an electrode array insertion device, such as those detailed in one or more of the above noted publications, or otherwise an arrangement that can read or otherwise determine an amount of array and/or a speed and/or a rate that the array is inserted in the cochlea. The sensor can provide output to a computer via a wired or wireless transmission system. Because speed and/or rate of insertion has a spatial component, that is included within “spatially based.” Further, an imaging system or an arrangement that can determine a pose or otherwise an orientation of the electrode array can provide the information to this computer system. Some additional details of these arrangements are described below. In an embodiment, the action of providing information to the computer system can be done in an audible manner, such as, for example, the surgeon or healthcare professional “reading out” or otherwise declaring how much of the electrode array has been inserted in the cochlea. This can be done based on the surgeon's (any disclosure herein of a surgeon corresponds to an alternate embodiment where some other type of healthcare professional is implementing the action and/or vice versa, providing that the art enables such and such has utilitarian value) ability to “estimate” the amount of array that has been inserted into the cochlea with or without the aid of markers on the electrode array. In an embodiment, the array could have indicators that can provide an indication as to how far the array has been inserted, such as an indicator that is present every three or five mm, and/or in an exemplary embodiment, the surgeon can count electrodes that have been inserted into the cochlea or otherwise can no longer be seen and/or otherwise still can be seen, and then subtracted from the total electrodes (e.g., if seven electrodes can be seen, the surgeon could call out seven and the computer system could determine that 15 electrodes have been inserted because the electrode array is a 22 electrode contact electrode array). And in this regard, an aide or a nurse or a helper could type in the information from the surgeon. In an embodiment, there is a computer system that includes a microphone that captures sound, and this microphone has a voice to text capability or voice to digital capability that can deduce what the surgeon is saying and convert it to numerical format or a computer readable format. In any event, the action of talking into the speaker and the speaker transducing the sound into an electronic signal for example, which signal is provided to the computer, corresponds to providing information to a computer system. Any device, system, and/or method that can enable the provided information to be provided to a computer can be utilized in at least some exemplary embodiments.

In an embodiment, the provided information to the computer includes temporally linked spatial based data relating to the electrode array during implantation procedure of the array. In this regard, the information could be a distance correlated to a time data set. By way of example only and not by way of limitation, in an embodiment, the aforementioned insertion device could have a computer system or a subsystem of its own with a timer where the output is correlated to time. Also, this could also be computed by a smart implant if powered up during surgery (such as for a totally implantable cochlear implant or a conventional implant normally powered from an external coil). Either could have a radio link to a smart phone or tablet or some other computing device in the operating room, for example. Computing could also be done in a cloud based system for example. (Any computational actions herein can be done in a could based system providing that the art enables such.)

The output could be data packets having distance correlated to time which is provided to the computer system. Conversely, in some embodiments, it is the computer system that correlates the input which can be spatial input only, with time. This could be more utilitarian with respect to the devices and systems that utilize automated sensors (where there is little to no time lag between the recording at the sensor and the receipt of the electrical signal or data set at the computer system in an exemplary embodiment) as opposed to the aforementioned verbal calling out of distance. In an embodiment, method action 1020 is executed in real time with the insertion process/the implantation process. In an embodiment, receipt by the computer system occurs within 5, 4, 3, 2, or 1 or less seconds from the actual spatial feature of the electrode array (that is, if the array is inserted 3.7 mm, the computer system receives the data corresponding to such within for example, two seconds).

In an embodiment, the action of method action 1020 can correspond to providing data over a server or over a cell phone connection or over a telephone connection, landline or otherwise, to a remote computer system. In an embodiment, the computer system can be at least tens or hundreds or thousands of miles away from the actor who is executing method action 1020. In an exemplary embodiment, the actor of method action 1020 acts to execute the method action by inputting this data into a local computing device, where that computing device can transfer the data or otherwise provide data based on the data that is inputted into the local computing device to the computer system. That said, in an exemplary embodiment, the computer system can be located in the surgical room where the implantation process is being executed, or can be located in another room of the hospital for example.

As will be described in greater detail below, the computer system is configured, in at least some embodiments, to evaluate the provided information or otherwise analyze the provided information for utilitarian effect. In this regard, in an exemplary embodiment, method 1000 includes the method action 1030, which includes the action of receiving information based on an evaluation by the computer system of the provided information, the evaluation having used the spatial based data to estimate a feature of interest of the cavity in the human, the received information being an indication of proximity between the electrode array and the feature of the cavity.

In an embodiment, there is a method action that entails moving the electrode array or maintaining the electrode array still according to the received information. In the aforementioned example, this would be stopping movement, and thus maintaining the electrode array still, such as for example, by any of the pause times detailed herein. The movement could be controlled by a system of which the computer system is a part. For example, the computer system could be a subsystem of a robotic system, where the received information is received by a microprocessor that controls the actuator of the robot. However, before such movement is executed, the surgeon or healthcare professional would approve such, by affirmative action, consistent with the fact that some of the actions herein are recommendations.

In an exemplary embodiment, where for example the feature of interest of the cavity in the human is a modiolus wall of a cochlea of the human, the received information could indicate that the electrode array is clinically against the modiolus wall. By “clinically against,” it is meant that the array is sufficiently close to the modiolus that for all practical purposes, it would be considered against the modiolus wall of the cochlea with respect to imaging or otherwise with respect to performing an analysis of the effects of the cochlear implant electrode array. Thus, “clinically against” includes in direct contact with the wall and also offset by a de minimus amount.

In an exemplary embodiment, the received information is distance data between the electrode array and the modiolus wall. In an exemplary embodiment, the received information is distance data between the electrode array and the estimated modiolus wall, obtainable by the teachings herein. That said, in an exemplary embodiment, the received information could be a color-coded image that shows different levels of distance between the electrode array and the wall. In an exemplary embodiment, the information could be an exaggerated graphic showing the locations where the electrode array is farther from the wall relative to other locations. A logarithmic scale can be utilized for example.

In an exemplary embodiment, the received information could also include recommended actions, such as whether or not to move the array or otherwise reposition or attempt to reposition the array. In an embodiment, the received information could be the amounts to move the electrode array further into the cochlea. In this regard, in an exemplary embodiment, the received information could indicate to move the electrode array by an amount or less than 15, 10, 9, 8, 7, 6, 5, 4, 3, 2, or 1 mm, or any value or range of values therebetween in a half millimeter increment. And note that this could be preference by the qualifier “about.” The exact measurements will not be utilized in an exemplary embodiment where the surgeon is manually inserting the electrode array. Conversely, exact amounts could be directed towards an arrangement where the actuator of the robot is moving the array.

The received information could be to not move the electrode array or otherwise stop moving the electrode array. This could be because the electrode array is fully inserted into the cochlea for example. This could also be because the history indicates that the prior movement forward caused the electrode array to move to a location where the electrode array was no longer clinically against the modiolus wall. Accordingly, the computer system could determine that based on the history, further movement will cause the array to move away from the wall, which in some embodiments is not desired (for example, some cochlea are obstructed by growths or other anatomical abnormalities, and there are few ways around this, and the computer will make a determination that as a matter of statistics, this is the best positioning that can be achieved, even though the array is not as far into the cochlea as would be desired), and therefore, the system will recommend that no further insertion be implemented.

Further, in an exemplary embodiment, the received information is also based on an evaluation by the computer system of the provided information, the evaluation having taken into account current spatial based data of the array. This is distinguished from, for example, history. In the context as used herein, the phrase history does not include current status. But note that the current status could be the same as a historical status such as where, for example, the electrode array has not moved since “historical times.” In an exemplary embodiment, a current pose is that a mid-section of the electrode array is clinically away from a modiolus wall of the cochlea. And in this method, in an exemplary embodiment, the received information is to move the electrode array backwards. This is done, because, based on the algorithm or otherwise the lookup table of the computer system, in these situations, based on the historical data and the current pose, there has been utilitarian value in the past with respect to withdrawing the electrode array a certain amount or otherwise pulling the electrode array backwards. The innovations associated with developing this database or otherwise a model that is utilized by the computer system will be described below.

With respect to the scenario where the mid-section of the electrode array is clinically away from the modiolus wall of the cochlea, this could be determined, including determined automatically (e.g., an average of the distances of the electrode array from the modiolus wall can be developed (e.g., the maximum distance in 0.5 mm segments of the array can be added together and divided by the total number of segments) and if the average exceeds a value, this could indicate that the mid-section is not clinically against the modiolus wall, or raw distances can be obtained, and if there is a single distance or two or more or three or more distances that exceed a certain value, it can be determined that the electrode array is not clinically against the modiolus wall, etc.) or manually (e.g., by assessing the numbers and making an “educated judgement” or by assessing a color-coded arrangement, etc.). In an embodiment where the average distance (mean, median, and/or mode) is less than, greater than and/or equal to 0.1, 0.125, 0.15, 0.175, 0.2, 0.25, 0.3, 0.35, 0.4, 0.45, 0.5, 0.6, 0.7, 0.8, 0.9, 1, 1.25, 1.5, 1.75, or 2 millimeters, or any value or range of values therebetween in 0.005 millimeter increments, over a distance of 1, 2, 3, 4, 5, 6, 7, 8, 9, 10, 11, 12, 13, 14, 15 mm or the number of electrodes, such can be an indication that the electrode array warrants repositioning in some instances (and not in some others). In an embodiment, the algorithms or the like can be configured to determine that if the average distance is greater than and/or equal to any one of the just detailed values (e.g., 0.155 mm, 0.0170 mm, etc.), an action should be taken to move the electrode array closer to the wall (which could be to withdraw the array a certain amount) or stop inserting or otherwise that this is an unacceptable distance away from the wall. In an embodiment, the algorithms or the like can be configured to determine that if the average distance is less than and/or equal to any one of the just detailed values, an action should be taken to continue to move the electrode array forward or otherwise stop moving the electrode array forward or otherwise that this is an acceptable distance from the wall. Note that these values may or may not be “clinically against” values. It could be that even if the array is not clinically against the wall, that is acceptable. Indeed, it could be that the history indicates that it is impossible or otherwise not feasible to have the array clinically against the wall. In this regard, the contour of the modular wall varies from patient to patient and a pre-curved electrode array is designed for the average patient. Accordingly, there will be instances where the design of the electrode array will simply be incompatible with a certain patient because the design is for the average patient and this particular patient could deviate from the average person's anatomy by a sufficient amount which would otherwise prevent the array from being against the modiolus wall. Accordingly, the algorithms of the computer system or the models of the system could be configured to determine that this is good enough or otherwise the best that is going to be achieved under the circumstances, without taking some other action that might not be desirable.

In an embodiment, the electrode array includes a tip portion at a distal end of the electrode array (which can be the portion that includes the radiopaque marker 838, or one or more of the electrodes as noted above), and the action of advancing includes moving the tip portion so that the tip portion is in contact with the wall of the cavity for at least about 20, 25, 30, 35, 40, 45, 50, 55, 60, 65, 70, 75, 90, 85, or 90% or any value or range of values therebetween in 1% increments of the distance that the electrode array is inserted into the cavity upon completion of the implantation procedure (i.e., measure the total distance of the array in the cavity, and the tip portion was in contact with the modiolus wall for a distance that is at least 70% of that total distance for example. In an embodiment, the tip portion includes the forwardmost portion (distalmost portion) of the array that is less than, greater than and/or equal to 0.5, 1, 1.5, 2, 2.5 or 3 mm or any value or range of values therebetween in 0.1 mm increments from the most forward (distal) portion of the array. In an embodiment, the tip portion is the portion that is within 0.25, 0.5, 0.75, 1, 1.25 or 1.5 mm from a geometric center of the radiopaque marker 838 or the distalmost contact (or up until the end of the array).

In an embodiment, the spatial based data includes a plurality of positions of a portion of the electrode array at different temporal locations during the action of advancing and/or a plurality of portions of respective different portions of the electrode array, the evaluation used the plurality of positions to develop a virtual model of the wall and/or a dataset representative of the wall and the received indication is based on the distance between another portion of the electrode array and the virtual model at a temporal location after the different temporal locations.

In an embodiment, the spatial based data includes a plurality of positions of a portion of the electrode array at different temporal locations during the action of advancing (e.g., the tip portion as it moves forward in the scala), the evaluation used the plurality of positions of the portion to develop a virtual model of the wall and the received indication is based on the distance between another portion of the electrode array and the virtual model at a temporal location after the different temporal locations. In an embodiment, the spatial based data includes a plurality of positions of respective different portions (e.g., the marker, electrode 3, electrode 5, electrode 10, electrode 15, etc.) of the electrode array during the action of advancing, the evaluation used the plurality of positions of respective different portions to develop the virtual model and/or dataset of the wall, and the received indication is based on the distance between some portion of the electrode array and the virtual model, the some portion corresponding to the different portions or another portion of the electrode array.

In an embodiment, at least one of: (i) the spatial based data includes a plurality of positions of a portion of the electrode array at different temporal locations during the action of advancing; or (ii) the spatial based data includes a plurality of positions of respective different portions of the electrode array during the action of advancing. In an embodiment, the evaluation used the plurality of positions of the portion and/or the plurality of positions of respective different portions to develop a virtual model of the wall. In an embodiment, the received indication is based on the distance between some portion of the electrode array and the virtual model, the some portion corresponding to the different portions or another portion of the electrode array.

In an embodiment, the spatial based data includes a plurality of positions (such as any of the number of positions detailed above) of a portion of the electrode array at different temporal locations during the action of advancing, the method includes developing a digital estimate of the wall based on the plurality of positions by treating the plurality of positions as latent variables indicative of position of the wall, and received indication is based on the distance between another portion of the electrode array and the digital estimate of the wall at a temporal location after the different temporal locations.

In an embodiment, the spatial based data includes a plurality of positions of a first portion of the electrode array at different temporal locations during the action of advancing and/or a plurality of positions of respective different second portions of the electrode array during the action of advancing. In an embodiment, the method includes developing a digital estimate of the wall based on one or both of the plurality of positions by treating the one or both of the plurality of positions as latent variables indicative of position of the wall. In an embodiment, the received indication is based on at least one of:
(i) the distance between another portion of the electrode array different from the first portion of the electrode array and the digital estimate of the wall at a temporal location after the different temporal locations; or
(ii) the distance between one or more of the first portion and/or the another portion and/or one or more of the second portions and the digital estimate of the wall.

In an embodiment, method 1000 includes moving the electrode array based on the received information. This could be a repositioning attempt as noted herein. The method includes providing second information after the action of moving, the provided second information including spatial based data relating to the electrode array during the implantation procedure of the electrode array into the human after the action of moving the electrode array. The method also can include receiving second information after the action of providing second information, the received second information being based on a second evaluation by the computer system of the provided second information, the second evaluation having used the estimated location of the wall and the spatial based data of the second information to estimate a location of the electrode array relative to the wall. The method can then include the action of advancing the array and then moving the electrode array backwards based on the received second information. The method can include receiving a third information to leave the electrode array at the location where it was moved backwards, and leaving the electrode array at the location where it was moved backwards based on the third information. Note that there could be intervening evaluations and informations (unless otherwise noted, the numerical indicators are simply for naming purposes, and thus a third X could come before a second X, etc.).

In an embodiment, the method includes moving the electrode array based on the received information, providing second information after the action of moving, the provided second information including spatial based data relating to the electrode array during the implantation procedure of the electrode array into the human after the action of moving the electrode array, receiving second information after the action of providing second information, the received second information being based on a second evaluation by the computer system of the provided second information, the second evaluation having used the estimated location of the wall and the spatial based data of the second information to estimate a location of the electrode array relative to the wall, advancing the array based on the received second information, providing third information after the action of advancing the array, the provided third information including spatial based data relating to the electrode array during the implantation procedure of the electrode array into the human after the action of advancing the electrode array, receiving third information after the action of providing third information, the received third information being based on a third evaluation by the computer system of the provided third information, the third evaluation having used the estimated location of the wall and the spatial based data of the third information to estimate a location of the electrode array relative to the wall and leaving the electrode array at the location where it was moved backwards based on the third information.

Embodiments also include computer readable media. In an embodiment, there is a non-transitory computer readable medium having recorded thereon, a computer program for executing at least a portion of a method, the computer program including code for receiving input based on at least one of (i) imaging of a cavity of a human and at least a portion of a medical device in the cavity during a first temporal period or (ii) electrical measurements taken by the at least a portion of the medical device in the cavity during the first temporal period. It is briefly noted that any disclosure herein of a method action or functionality corresponds to an alternate disclosure in the interest of textual economy of a disclosure for a computer readable medium, and vice versa, providing that the art enables such, unless otherwise noted. Accordingly, the just noted disclosure of the code corresponds to the alternate disclosure of receiving input based on at least one of imaging of a cavity of a human, etc.

In an embodiment, the media also includes code for automatically analyzing the received input to develop data indicative of an estimated boundary of the cavity. This could be a virtual model of the wall, such as the modiolus wall, or could be a data set of numerical values that are utilized to represent a boundary condition in further calculations.

The media also includes one or both of:
(a) code for determining a position of at least another portion of the medical device relative to the estimated boundary based on the received input; or
(b) code for receiving second input based on at least one of (i) second imaging of the cavity of the human and the at least another portion of the medical device or another medical device in the cavity during a second temporal period following the first temporal period or (ii) electrical measurements taken by the at least another portion of the medical device or the another medical device in the cavity during the second temporal period and code for determining the position of the at least another portion of the medical device or the another medical device relative to the estimated boundary based on one or both of the received input or the received second input.

With respect to the code of “a,” this could be a code that creates a plurality of tangent surfaces at predetermined intervals or non-predetermined intervals along the virtual surface of the modiolus wall and utilizes a standard computer algorithm to determine the distance from the tangent surface to the another portion of the medical device, such as, for example, electrode 10. This can be standard technology which applies the two closest points in a virtual model, not unlike the technology utilized in the movie “Italian Job” to determine the height of the armored car body relative to the tires, where the known dimensions of known references are utilized, or otherwise the imaging is scaled so as to determine the distance. Instead of tangent planes, a spread of dimensions sub tending 180° over the surface of 10 for example facing the modiolus wall can be acquired, and the closest dimension or more accurately, the smallest dimension, between the electrode 10 and the surface of the modiolus wall can electrode be utilized as the distance, and thus can be utilized to determine the position of the at least another portion of the medical device relative to the estimated boundary. Standard computer-aided design modeling techniques can be utilized.

Note the concept of the at least another portion of the medical device or another medical device. Again, with reference to the concept of utilizing a probe, the “another medical device could be the cochlear implant electrode array, where the probe was utilized to develop the data set or the virtual model for the modiolus wall.

With respect to the code of “b,” this can be based on second imaging of the cavity of the human and the at least another portion of the medical device as just noted. In an embodiment, the second imaging can be taken after the first imaging with the imaging precedent. Here, the images can be compared to each other, and reference structure of the cavity, such as the lateral wall which is readily imaged or otherwise much better imaged than other components of the cavity, can be utilized to establish a common frame of reference so as to match up the estimated location of the modiolus wall in the two images. This could be done by estimating the distances from the lateral wall to the estimated location of the modiolus wall in the first set of images, and then utilizing those distances to establish the location of the modiolus wall or more accurately, the estimated location of the modiolus wall, and the second set of images. For example, a computer algorithm can take the tangent lines of the surface of the lateral wall facing the modiolus wall, and utilize directions normal to those tangent lines to “computationally pace out” the distances to the modiolus wall (the estimated location thereof) thereby establishing the estimated location and the second set of images. This concept can be utilized in reverse for the first set of images (or not—the normal directions to the tangent lines can be utilized as the reference and the modiolus wall, the estimated location of the modiolus wall, can be superimposed over those normal directions and then the computer system can determine the distances from the tangent lines at the specific locations on the lateral wall to the estimated location of the modiolus wall, or vice versa. In an embodiment, there are less than greater than and/or equal to 5, 6, 7, 8, 9, 10, 11, 12, 13, 14, 15, 16, 17, 18, 19, 20, 25, 30, 35, 40, 45, 50, 55, 60, 70, 80, 90, 100, 150, 200, 250, 300, 400, 500, 600, 700, 800, 900, or 1000 or more or any value or range of values therebetween in one increments tangent lines and/or distance is normal to those tangent lines or otherwise data points on the image of the lateral wall that are utilized as a reference to “place,” the estimated location of the modiolus wall virtually, and vice versa (there could be, for example, 223 data points are tangent lines or normal directions from the estimated location of the modiolus wall to the lateral wall, and note that the numbers need not be the same with respect to the lateral wall and the modiolus wall). The algorithm can utilize this numerical technique to place the various features relative to known features. In a similar vein, the algorithm can utilize the images of the medical device of the other medical device and utilize the just detailed techniques to determine the distances from the lateral wall or from the modiolus wall, or otherwise place the portion of the medical device or other medical device at interest relative to the estimated location of the modiolus wall or the lateral wall, where the latter can be utilized as a reference for all the other elements as the lateral wall is a stable reference point.

Note also the option of utilizing electrical measurements taken by the at least another portion of the medical device or the another medical device located in the cavity relative to the estimated boundary based on one or both of the received input with a receipt second input. As noted above, electrical measurements can be utilized to determine locations of one or more of the structures and/or components detailed herein, at least location relative to one another. The computer can read the resulting impedances between electrodes for example, which impedances can change depending on the distance to the modiolus wall of the cochlea, and utilizing those impedances, or more specifically, comparing those impedances to a reference set of impedances obtained from statistically similar implantation scenarios/prior patients where the distances were known, and location of the modiolus wall can be estimated based on the comparison, where, for example, the algorithm can look at and impedance value of I for example and correlate that to a distance from the electrodes to the modiolus wall, and then estimate the location of the modiolus wall, and this estimate of the location can be relative to a known structure, such as the lateral wall, and note that in some embodiments, it is not required to determine the location if, for example, the distance is known because in at least some exemplary embodiments, it is the distance between the electrodes and the modiolus wall that is utilitarian to determine whether or not an acceptable placement of the electrode array has been achieved.

In an embodiment, if the code for receiving input based on at least the imaging during the first temporal period is present, the imaging during the first temporal period includes imaging of the cavity and the at least a portion of the medical device at different locations in the cavity and if the code for receiving input based on at least the electrical measurements taken during the first temporal period is present, the electrical measurements taken during the first temporal period includes measurements taken by the medical device at different locations in the cavity.

In an embodiment, the medium includes the code for determining the position of the at least another portion of the medical device relative to the estimated boundary based on the received input, and the code for determining the position of the at least another portion of the medical device relative to the estimated boundary based on the received input does so based on the imaging and/or the electrical measurements taken with the medical device at different locations in the cavity. In an embodiment, there can be less than, greater than and/or equal to 2 (and thus 1 if less than), 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200, 250, 300, 350, 400, 450, 500, 600, 700, 800, 900, 1000, 1500, 2000, 2500, or 3000 or more, or any value or range of values therebetween in one increment different locations. And in an embodiment, these different locations can be utilized to establish a virtual image of the medical device relative to the modiolus wall which can be displayed on a computer screen or in an optical microscope so as to provide an indication to the surgeon of the relative locations of the electrode array relative the modiolus wall.

In an embodiment, the different locations in the cavity correspond to locations of advancement of the medical device into the cavity. In an embodiment, the different locations correspond to increments of less than greater than and/or equal to 0.5, 1, 1.5, 2, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, or 20 mm, or any value or range of values therebetween in 0.1 mm increments (and the increments may not be the same or do not need to be the same, so the first increment could be less than 0.5 mm and the second increment could be greater than 0.7 mm and the third increment could be 1.3 mm and the fourth could be 0.5 mm, etc.). Corollary to this is that in an embodiment, the different locations correspond to increments of less than greater than and/or equal to 0.5, 1, 1.5, 2, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, or 20 degrees, or any value or range of values therebetween in 0.1° increments, and again, the increments may not be the same and do not need to be the same.

In an embodiment, such as where the cavity is a cochlea, the different locations in the cavity include at least and/or equal to and/or no more than 1, 2, 3, 4, 5, 6, 7, 8, 9, or 10 locations or any value or range of values therebetween in one increment, and the locations include at least a location at or greater than and/or less than and/or equal to 5, 10, 15, or 20 degrees of insertion, at least a location at or greater than less than and/or equal to 10, 15, 20 or 25 or 30 or 35 degrees of insertion, at least a location at or greater than less than and/or equal to 20, 25, 30, 35, 40, 45, 50, 55, 60, 65 degrees of insertion and at least a location at or greater than less than and/or equal to 35, 40, 45, 50, 55, 60, 65, 70, 75, 80, 85, 90, 95, or 100 degrees of insertion or any value or range of values there between the just detailed number of degrees in 1° increments. In an embodiment, each one of the aforementioned insertion values is greater than the prior value.

In an embodiment, such as where the cavity is a cochlea, the different locations in the cavity include at least and/or equal to and/or no more than 1, 2, 3, 4, 5, 6, 7, 8, 9 or 10 locations or any value or range of values therebetween in one increment, and the locations include at least a location at or greater than or less than and/or equal to 3, 4, 5, 6, 7, 8, 9, 10 mm of insertion, at least a location at or greater than less than and/or equal to 5, 6, 7, 8, 9, 10, 11, 12, 13, 14 or 15 mm of insertion, at least a location at or greater than less than and/or equal to 8, 9, 10, 11, 12, 13, 14, 15, 16, 17, 18, 19 or 20 mm of insertion and at least a location at or greater than less than and/or equal 10, 11, 12, 13, 14, 15, 16, 17, 19, 19, 20, 21, 22, 23, 24 or 25 mm of insertion or any value or range of values there between the just detailed number of millimeters in 0.1 mm increments. In an embodiment, each one of the aforementioned insertion values is greater than the prior value.

In an embodiment, the cavity is a cochlea and the medical device is a cochlear implant electrode array and the imaging and/or electrical measurements include images and/or measurements with a tip portion of the cochlear implant electrode array against a modiolus wall of the cochlea at different locations along the modiolus wall, the tip portion corresponding to the at least one portion of the medical device. In an embodiment, at least where this disclosure corresponds to a method, the tip portion remains in contact with the modiolus wall for any one or more of the aft aforementioned contact regimes detailed above. In an exemplary embodiment, again where the cavity is a cochlea and the estimated boundary is a modiolus wall of the cochlea, the code for automatically analyzing the received input to develop and estimated boundary of the cavity includes code that uses a plurality of positions of the least one portion of the medical device during the first temporal period, which positions are based on the received input, to establish the estimated boundary of the cavity.

In an exemplary embodiment, the code for receiving second input based on at least one of (i) imaging of the cavity of the human and the at least another portion of a medical device or another medical device in the cavity during a second temporal period following the first temporal period or (ii) electrical measurements taken by the at least another portion of the medical device or the another medical device in the cavity during the second temporal period and code for determining the position of the at least another portion of the medical device relative or the another medical device relative to the to the estimated boundary based on one or both of the received input or the received second input. In an embodiment, the code for determining the position of the at least another portion of the medical device relative to the estimated boundary based on the received input does so based on the imaging and/or the electrical measurements taken with the medical device at different locations in the cavity.

Embodiments include repositioning the array based on the analysis of the location of the array or the portions thereof relative to the estimated location of the modiolus wall. In many embodiments, this can entail pulling the electrode away backwards or otherwise withdrawing the electrode away a certain amount, such as the amounts detailed above. This could have the results of pulling the array closer to the modiolus wall as noted above. This could be utilitarian with respect to the simple concept of trying to get the closest location possible to obtain the best spatial data that will be utilized to estimate the location the modiolus wall. That is, even though it might not be desirable to have the electrode array withdrawn by that amount, the purpose of withdrawing the electrode array by that amount is for the estimation purposes and not necessarily the ultimate placement purposes. Accordingly, embodiments can include withdrawing the electrode array by certain amount, including not fully withdrawing the electrode array from the cochlea, and then applying the imaging or the measuring techniques detailed herein under the premise that the array is as close to the modiolus wall as possible because of the movement backwards or otherwise because of the tension applied to the electrode array in the opposite direction of the force that is applied during the insertion process, and then applying a forward force or otherwise reinserting at least a portion of the part that was withdrawn into the cochlea. Corollary to this is that such actions can also be utilized as part of the “reseating process” where based on the analysis of the distance of the electrode array to the modiolus wall, it is deemed utilitarian to try to get the electrode array closer to the modiolus wall, and such could entail pulling the electrode array backwards a bit and then driving the electrode array forward and then taking new images or new electrical measurements to determine whether or not the electrode array is any closer to the modiolus wall or at least to determine that it is not further from the modiolus wall relative to that which was previously the case.

In an embodiment, there are a number of ways to withdraw the electrode array. For example, if the electrode array is still in the sheath, then one can pull the electrode array back through the sheath, or one can pull the sheath and electrode out together. These might have different effects depending on how far the electrode is inserted. Of course, if a sheath is not being used, one can pull the array by itself. Also, if the sheath has already been removed, then one can pull the electrode back with no sheath involved. Accordingly, in some embodiments, the recommendations/instructions can include any one or more of the aforementioned withdrawal techniques. Moreover, based on the data collected, the system could advise/recommend to remove the electrode array completely, reload into the sheath (if a sheath is being used), and start afresh. Accordingly, embodiments herein can include recommending the relationships of movements relative to the insertion sheath and/or can include completely starting over, whether such is with the use of the sheath or not. Indeed, it could be that a determination is made that the insertion sheath is not useful with respect to a particular insertion, and thus the system could recommend to not use the sheath/stop using the sheath, or alternatively, could recommend to use and insertion sheath if the procedure up until that point has been utilized without one

The contour an electrode array will take will also depend, in some embodiments, on the current location of the array in the cochlea. For example, if the array is resting on the modiolus, or flexed away from the modiolus, or at a point in the insertion where the tip has previously hung up on the modiolus, the next movement can result in a different contour. (Note that in various scenarios, the next movement may result in a fully inserted or partially inserted array.) Accordingly, embodiments can include moving the electrode array so as to get the electrode array closer to the modiolus wall for the estimation purposes and/or for ultimate placement purposes. And to be clear, embodiments include scenarios where the spatial data of the components of the electrode array that is utilized to develop the model of the wall are so problematic that it produces a model of the wall that is clearly erroneous. In this regard, in an exemplary embodiment, the resulting model can be utilized not only to estimate the location of the modiolus wall, but as a proxy for a “bad” electrode array insertion or placement. In an embodiment, if the resulting model or data set or what have you is sufficiently aberrant from what would be normal as a matter of statistics, the system could automatically indicate that there is a bad placement or bad seating or otherwise that the surgeon should evaluate the placement or otherwise make additional efforts to verify that the electrode array is indeed correctly placed.

Embodiments include an automated arrangement that provides an indication that informs the surgeon on how to make and/or provides additional information to make a decision about the next movement of the electrode array. And as will be described below, embodiments can include communicating with the implantable component and/or the external components of the hearing prostheses for example, and in an embodiment, data could be stored in a smart implant and the implant itself could contribute to the analysis. The smart implant can have a direct Bluetooth communication with an external device which could be a standard smart phone or tablet, by way of example, or any of the components herein. In an embodiment, it is the implant, or the external component of the prostheses or both, that performs the analysis or otherwise makes the recommendation, or at least performs part of the analysis. Any disclosure herein of a system that performs the analysis or otherwise evaluate the data or otherwise collects data or otherwise records the data or otherwise stores the data corresponds to a disclosure in the interest of textual economy of the implant and/or the external component doing so providing that the art enables such unless otherwise noted.

By way of exemplary scenario, the electrode array is practically fully inserted, or otherwise is more than a certain amount inserted (e.g., at least 60, 65, 70, 75, 80, 85, 90, or 95% of the length of the array that will ultimately be inserted at completion of the insertion process is inserted, or any value or range of values therebetween in 1% increments) and the most recent action was to insert further, and there is evidence that the mid-section of the array has moved away from the modiolus (which indication could be by way of the wall modeling/estimating techniques detailed above, which could use the CT scan (cone or otherwise) and/or electrical energizement of electrodes, or by the any one or more of the above-referenced patent documents—in an embodiment, the teachings of those publications can be used to determine the spatial features and/or movement features detailed herein and/or any spatial feature and/or movement feature that can be determined using the teachings of those references can be used herein for the spatial features and/or movement features). In an embodiment, the electrodes of the electrode array can be energized and read electrodes thereof can be used to sense spatial features/movement features, and this sensed data can be provided to the systems disclosed herein—more on this below.

Embodiments can include breaking up the insertion into phases. A first phase could cover the introduction of the sheath to an optimal depth and angle to facilitate advancing the electrode out of the sheath with the optimal vector to avoid going too wide of the modiolus. The second phase could cover the advancement of the electrode array until it has reached the full depth of insertion and ensuring during this phase that the electrode array does not bow outward and cause trauma to the lateral wall on the way, and embodiments herein can be directed to avoiding such (damage to the lateral wall), and reversing the bowing/eliminating such if such occurs. And in the third phase, the array is at or around full insertion, and this phase can including optimizing/perfecting as best possible the final position of the electrode array as close as possible to the modiolus. During phase 2, a goal can be to avoid the electrode array impinging on the lateral wall, or at least limiting the amount of force thereon. In some embodiments, it is not essential to be as close as possible to the modiolus, however being far away from the lateral wall (and hence close as possible to the modiolus) gives more margin for error and room for adjustment of the insertion movements. During phase 3, a goal can be to have the array end up as close as possible to the modiolus, and while attempting to execute this, avoid pushing out onto the lateral wall. Embodiments, can thus include recommending movements and actions etc., including the application of force or the reduction of force onto the array in the direction of insertion and/or in the direction of withdrawal, so as to avoid the array impinging on the lateral wall and/or to reduce the likelihood of such and/or at least reduce the amount of force applied to the lateral wall relative to that which would otherwise be the case. Embodiments thus can include devices systems and methods that can forecast or otherwise estimate what actions would result in the array moving towards the lateral wall or contacting the lateral wall or increase the force onto the lateral wall or otherwise results in a force that is unacceptably high, and thus recommend actions to take that would reduce the likelihood or otherwise avoid such. Embodiments can also include devices systems and methods that can recommend what actions to take to keep the array is closed as possible to the modiolus and/or avoid pushing the array out onto the lateral wall. Embodiments can thus include devices systems and methods that can forecast what actions would result in the array moving away from the modiolus or otherwise pushing the array outward onto the lateral wall and thus recommend actions to take that would reduce the likelihood or otherwise avoid such and what actions to take that will result in the array being as close as possible to the modiolus wall. In any one or more or all of these phases, new data can be obtained, such as new spatial data, and the spatial data can be utilized to evaluate the relative locations of portions of the electrode array relative to the estimated location of the modiolus wall, which evaluation can be utilized to determine features associated with the current placement of the electrode array so as to determine whether or not an additional change should be attempted or otherwise some additional action should be taken so as to improve the placement of the electrode array.

Embodiments can break down, for evaluation purposes, in real time, literally or computationally (e.g., akin to a finite element analysis of a circle or a curved surface (a series of facets—in some embodiments, so minute that for all practical purposes, the facets are indistinguishable from a smooth surface)), the movement of the electrode array in relatively discrete and small steps and can correlate this data to the estimated location of the modiolus wall so as to develop distance and/or location of data. Embodiments can then use this breakdown to analyze the past and the present status of the array, and automatically deduce a recommended next action. By way of example, if the electrode array is practically fully inserted and/or inserted by any of the amounts noted above for example, and the most recent action was to insert the array further and there is reason to believe (evidence, or otherwise, such as because the algorithm used to analyze the status/pose indicates such, or indicates a possibility of such) that the mid-section of the array has moved away from the modiolus (in absolute terms, or by an amount that is deemed undesirable or otherwise problematic, or outside a predetermined tolerance), then the system/algorithm would potentially indicate, for example, that the next action should be to pull back the array and measure or evaluate if this brings the mid-section of the array closer. The system/algorithm could recommend that this could be repeated (insert a step and withdraw a step) to confirm and then leave the electrode in that place. The system could then make a determination that if, however, the electrode array is only half inserted, and there is an indication that the apical electrodes are moving away from the modiolus (based on the analysis), this could be due to the tip becoming caught on the modiolus (the analysis could automatically determine this based on inputted data). In this instance, for example, the system/algorithm could recommend that pulling back on the array should bring the apical electrodes and/or the mid electrodes closer to the modiolus. (Note also that for a pre-curved perimodiolar electrode array, it is possible for the tip to be stubbed into the modiolus and the most proximal section to the tip to be away from the modiolus.) In an embodiment, it is the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th} or 15^{th} or any one or more of those electrodes, where the most apical electrode is the first, that is moved towards the modiolus. However, in this exemplary scenario, the electrode array is only partly inserted, so the goal is to release the tip from being caught and then insert further, and the system/algorithm would determine that based on the data. In this instance, the system/algorithm could determine that it is utilitarian to withdraw the electrode array by a significant distance until there is evidence that the tip is starting to pull back out, thus indicating (based on the algorithm/analysis) that the tip is released from whatever has caught the tip. The system could then analyze the data (where, in some embodiments, the data is constantly updated or otherwise updated at least every 5, 4, 3.5, 3, 2.5, 2, 1.5, 1, 0.9, 0.8, 0.7, 0.6, 0.5, 0.4, 0.3, 0.2, 0.1, 0.075, 0.05, 0.025, 0.02, 0.01 seconds or less, or any value or range of values therebetween in 1 millisecond increments), and after this, based thereon, indicate that the electrode array should be pushed further to get the tip past the stoppage. In an embodiment, the system could evaluate the location of the array relative to the modiolus upon the further pushing, and ratification of this instruction could be indicated by the electrode array going deeper without pushing out from the modiolus (at least within the tolerances).

In an exemplary embodiment, upon an action to reduce or otherwise eliminate the separation of the electrode array from the modiolus wall and/or a determination that the electrode array has moved away from the modiolus wall by a certain amount, such as by an amount that renders the array no longer clinically against the modiolus wall, and/or by an amount specified, such as for portions of the electrode array that are away from the modiolus wall (not just clinically away), the average distance (mean, median and/or mode) is less than, greater than and/or equal to 0.1, 0.125, 0.15, 0.175, 0.2, 0.25, 0.3, 0.35, 0.4, 0.45, 0.5, 0.6, 0.7, 0.8, 0.9, 1, 1.25, 1.5, 1.75 or 2 millimeters or any value or range of values therebetween in 0.005 millimeter increments, further advancement of the electrode array does not increase the average (mean, median and/or mode) separation distance by more than and/or equal to JKL %, where JKL is 5, 10, 15, 20, 25, 30, 40, 50, 60, 70, 80, 90, 100, 125, 150, 175, 200, 225, 250 or any value or range of values therebetween in 1% increments. In an embodiment, the computer system estimates or otherwise predicts that this will be the case, and then recommends the further movement into the cochlea of the electrode array. In the verification or validation that the above parameters were met or are Matt can be achieved by comparing the location of the array to the estimated location of the modiolus wall.

In an embodiment, the action of moving the electrode array according to the received information is executed using an automated insertion component automatically controlled based on the received information. Additional details of this will be described below, but, briefly, in an exemplary embodiment, there can be an automated handheld actuation device such as an electrode guide with rollers that advance and/or retract the electrode array into the cochlea based on a received signal. This handheld actuation device can be handheld by a surgeon or other healthcare professional during the surgery. In an exemplary embodiment, the actuation device operates based on control signals from the computer system or of the computer system, but is subject to actuation only upon the approval of the surgeon. In other embodiments, the actuation device is controlled by the surgeon, such as with a toggle switch or the like, where the surgeon controls the actuation based on the received information.

Some embodiments can use the teachings herein to determine whether an electrode array becomes unusable (for a variety of reasons this can be the case) and it would be utilitarian for the surgeon to use another device (e.g., a backup device). The system could recommend such based on the data, and the system could also keep the history of the first device which failed to be inserted (or was not inserted) and refer to that history to advise on the insertion of a second device. The second device could be a different electrode array model (e.g., a straight array instead of a curved array, or another model of curved array, or visa-versa). The history could be collected in a traditional manner, such as by surgeons inputting data into a database or otherwise providing data to a central location where it is compiled, etc., or otherwise uploading the data to the cloud where it is compiled, consistent with the other data that is collected to establish the algorithms or otherwise train the neural network, or otherwise establish the statistical big data protocols, etc., Or the system could automatically log this and then update its own records or otherwise update the central database, etc. In some instances, sometimes the cochlea is obstructed somewhere beyond the visibility of the surgical microscope. In attempting to insert an electrode array to the full depth in such a scenario, the electrode array could become damaged and/or could damage the tissue of the cochlea. It could simply be that as a matter of statistics, it is essentially impossible or otherwise extremely unlikely that the array will ever be seated within the cochlea in a proper manner or otherwise in a manner that does not damage the tissue or the array with that particular electrode array. A second electrode array which could be the same or different type of electrode array, say a straight array instead of a curved array, or visa-versa, or a round cross-section array vs. a rectangular cross section array, or a styletted array vs. a styletless array, and all visa-versa, etc. Knowing the point of the apparent obstruction from the first insertion (based on latent variables, such as the insertion depth/insertion angle), the system could advise to slow down at that point and not push too hard or too far. If the second electrode array cannot get past the obstruction (or be further inserted than the first), the surgeon has the choice to leave the electrode array partly inserted, or perhaps to remove that electrode array before it is damaged and use a Contour depth gauge to open up the cochlea at the point of obstruction, or to take other actions that are typical solutions in the specific scenario. In an embodiment, the system can recommend one or more of these actions, and also this data can be collected and utilized to train the DNN and, etc., consistent with the teachings detailed herein. And all of this data can be collected/used during the insertion process to make the recommendations/forecasts herein.

In an exemplary embodiment, the analyzed data can include the current status of the electrode array, such as completely inserted into the cochlea or half inserted into the cochlea, or 7 mm thereof inserted into the cochlea, etc. The analyzed data can also include the past status of the electrode array. This can have utilitarian value with respect to the overall analysis by the computer system such as by linking the past status of the electrode array with a past pose of the electrode array, where the pairing can be utilized to more accurately predict how the electrode array might move in the future. By way of example, if the status of the array is only 1 or 2 mm inserted into the cochlea, and the pose is that there is a space between the modiolus wall and the electrode array, the system could determine that this is not a problem or otherwise should be ignored because this is what happens during the initial insertion process at least in some instances.

In an embodiment, the analyzed data includes at least GHI discretized movements based on movement(s) during a portion of the insertion process and the, the analyzed data includes at least GHI locations of the array during the first temporal period (and the values need not be the same—the variable GHI is used for textual economy), where GHI is 1, 2, 3, 4, 5, 6, 7, 8, 9, 10, 11, 12, 13, 14, 15, 16, 17, 18, 19, 20, 21, 22, 23, 24, or 25 or more, or any value or range of values therebetween in 0.25 mm increments. In an embodiment, the analyzed data includes at least GHI statuses of the array during the first temporal period. And note that the discretized movements can correspond to individual movements of the array made by the surgeon or the actuator. By way of example only and not by way of limitation, in an exemplary embodiment, the actuator that is utilized to insert the array moved to the array in a stepwise fashion, such as, for example, one ¼^{th} of a millimeter per second, or ½ of a millimeter or 1 mm for example. In an embodiment, the movement takes place within ¼ or ½ a second for example. The remainder of the time constitutes stationary time for the electrode array, at least relative to the insertion device/actuator or the cochlea. Corollary to this is that in at least some exemplary embodiments, the surgeon when hand driving the electrode array (e.g., the surgeon is pushing on the electrode array to drive the electrode array into the cochlea), could push the electrode array or otherwise move the electrode array two or three or four mm every two or three or four seconds for example. The movement could take place within a second or half a second. The remainder of the time is stationary time. And note that the time periods can be variable and can be different from one segment/step versus another. The point is that the discretization can be that which corresponds to real life discretization of the movements and/or can correspond to a division of a continuous movement into segments.

The concept of discretization can also be extended to different locations of different parts of the array relative to the modiolus wall. Accordingly, in an exemplary scenario, the insertion process can be discretized into tens or hundreds or thousands, tens of thousands or more “frames.” In an embodiment, any value or range of values of discretizations from 1 to 100,000 or more in 1 increment can be used in at least some exemplary embodiments.

In an embodiment, there is a system, such as the computer system detailed above, or another system, comprising an input suite configured to receive input based on a spatial feature of an implantable medical device during implantation into a human. This can be a server, or can be a USB sub-system, or can be a microphone or a video camera or still camera, consistent with the teachings above. In an embodiment, the input suite could be an insertion guide according to the teachings below, which insertion guide includes components that can determine the position of the electrode array. The input based on a spatial feature of the implantable medical device can be digital data or analog data, such as the output of the insertion guide, or can be an electrical signal or a magnetic signal, where the electrical signal corresponds to a change in voltage or current for example that results when an electrode of the electrode array passes by a contact. Other options can be, for example, the use of strain gauges within the electrode array or bragg gratings in an optical fibre in the electrode array that can provide information on the shape of the electrode array in real time or near real time. Any device, system, and/or method that can enable the system to receive input based on the spatial feature of the implantable medical device during implantation to be used in at least some exemplary embodiments.

The system also includes an output suite, which could be a server or a USB sub-system (where the input suite can be integrated with the output suite concomitant with the commonly implemented USB devices) or a speaker or a video monitor, etc. In an embodiment, the output suite could be the insertion guide according to the teachings below, which insertion guide includes components that move the array according to a received signal. The output of the output suite can be digital data or analog data, etc. Any device, system, and/or method that can enable the system to receive input based on the spatial feature of the implantable medical device during implantation to be used in at least some exemplary embodiments. The system also includes a data analysis computer in signal communication with the input suite and the output suite, the data analysis computer including circuitry and being configured to automatically analyze, at least in part with the circuitry, the received input as that input relates to the data herein associated with the medical device during implantation and automatically develop, based on the analysis, any one or more of the details detailed herein (actions or features, etc.). The system is configured to at least one of automatically output data based on the actions herein via the output suite or move the medical device based on the developed movement via the output suite. With respect to the former, this could be via a speaker in the operating room that provides an audible statement as to the developed subsequent movement, where the surgeon or other healthcare professional can evaluate the merits of such and act accordingly. Also with respect to the former, this could be providing a control signal or the like to a robot or the actuator of the insertion guide to control the insertion guide to move the array accordingly. With respect to the latter, this could be completed by movements of the robotic actuator.

Accordingly, in an embodiment, the system includes and/or is in signal communication with a robotic device that is configured to robotically move the medical device during implantation.

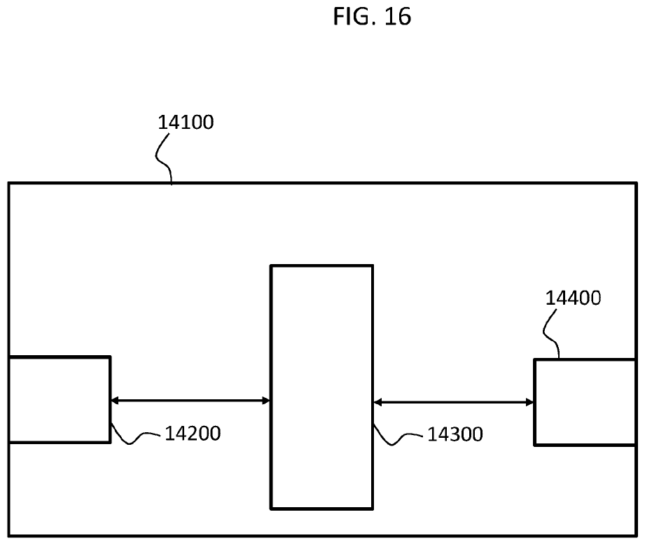

FIG. 16 shows the system 14100, with input suite 14200 and output suite 14400 in signal communication (e.g., by wires or by fiber optics, etc.) with the data analysis computer 14300.

Consistent with the teachings herein, there is a data analysis computer that is configured to automatically analyze the received input also as that input relates also to spatial features of the medical device and/or the imaging and/or the measurements. Any of the spatial features/imaging/measurements detailed herein can be used.

In some embodiments, the system includes and/or is in signal communication with an automated monitoring device that is configured to automatically monitor movements and/or position of the medical device during implantation, wherein the received input is based on output from the automated monitoring device. Additional details of this will be provided below, but briefly, in an embodiment, an insertion guide, in the case of a cochlear implant electrode array, can be equipped with an “electrode counter” that can determine when an electrode of the array moves past a sensor, and thus determine the depth of insertion based on the electrode that is at/has passed the sensor. In an embodiment, there can be an arrangement that measures the impedance change as electrodes move into the inner ear. Because the electrodes go from air to perilymph, one can detect a change in impedance from high to low impedance. This can be used to determine the insertion depth, or otherwise to “count electrodes.” This can be done with an implant that is configured to measure the impedance change, or the outright impedance for that matter.

Again, more on this below. Moreover, again by way of example where the medical device is a cochlear implant electrode array, in an exemplary embodiment, the system is configured to discretize the input data to establish discretized movement and/or positions of the array (as detailed above). Additionally, the system can be configured to use the discretized input data to establish the history of the spatial features of the medical device.

The data analysis computer can be implemented via a wide variety of regimes. In an embodiment, the data analysis computer includes electronics and/or software that is a product of and/or resulting from machine learning that is configured to execute the automatic analysis. More specifically, at least some exemplary embodiments according to the teachings detailed herein utilize advanced learning processing techniques, which are able to be trained to detect higher order, and non-linear, statistical properties of data. An exemplary analytical technique is the so called deep neural network (DNN). At least some exemplary embodiments utilize a DNN (or any other advanced learning analytical technique) to analyze the historical spatial features and/or current spatial features and/or other data relating to the medical device during implantation. At least some exemplary embodiments entail training data analysis algorithms/developing models to detect subtle and/or not-so-subtle changes, and provide an estimate of future statuses and conditions, etc., and specific information thereabout, that can correspond to how the array will move/the future spatial features of the array. That is, some exemplary methods utilize learning algorithms such as DNNs or any other algorithm that can have utilitarian value where that would otherwise enable the teachings detailed herein to analyze the data relating to the array to predict how the array will move and/or to determine how the array should be moved to achieve a utilitarian placement, etc.

A “neural network” is a specific type of machine learning system. Any disclosure herein of the species “neural network” constitutes a disclosure of the genus of a “machine learning system.” Moreover, any disclosure herein of the species “machine learning” constitutes a disclosure of the genus of “artificial intelligence.” While embodiments herein focus on the species of a neural network, it is noted that other embodiments can utilize other species of machine learning systems accordingly, or the broader genuses noted, any disclosure herein of a neural network constitutes a disclosure of any other species of machine learning system that can enable the teachings detailed herein and variations thereof. To be clear, at least some embodiments according to the teachings detailed herein are embodiments that have the ability to learn without being explicitly programmed. Accordingly, with respect to some embodiments, any disclosure herein of a device or system constitutes a disclosure of a device and/or system that has the ability to learn without being explicitly programmed, and any disclosure of a method constitutes actions that results in learning without being explicitly programmed for such.

Some of the specifics of the DNN utilized in some embodiments will be described below, including some exemplary processes to train such DNN. First, however, some of the exemplary methods of utilizing such a DNN (or any other system that can have utilitarian value) will be described.

It is noted that in at least some exemplary embodiments, the DNN or the product from machine learning, etc., or the results thereof, etc., is utilized to achieve a given functionality as detailed herein. In some instances, for purposes of linguistic economy, there will be disclosure of a device and/or a system that executes an action or the like, and in some instances structure that results in that action or enables the action to be executed. Any method action detailed herein or any functionality detailed herein or any structure that has functionality as disclosed herein corresponds to a disclosure in an alternate embodiment of a DNN or product or results from machine learning, etc., that when used, results in that functionality, unless otherwise noted or unless the art does not enable such.

Additional details of the DNN and the training thereof are provided below.

In an embodiment, by way of example only and not by limitation, the medical device is a cochlear implant electrode array, the data analysis computer is an artificial intelligence subsystem, which can be a neural network, such as a deep neural network.

The system of which the subsystem is a part can be configured to predict, with the use of the artificial intelligence subsystem, a next behavior of the array based on the input, or determine that the placement of the array is acceptable or unacceptable (or inconclusive). The system of which the subsystem is a part can be configured to determine, with the use of the artificial intelligence subsystem, the next movement of the array that should be taken based on the input, or that the array placement is good or not good, etc. (The subsystem can have “prediction model(s)” and/or “determination model(s)” that can be used to make the determinations and/or predictions. More on this below.) In this regard, different systems, which systems are not mutually exclusive, can be utilized for different purposes. On the one hand, the prediction of how the electrode array will move given the past history and/or the current features of the array can have utilitarian value with respect to providing information to the surgeon or other healthcare professional that will enable him or her to decide what action should be next taken, which could be to obtain the predicted next behavior, or to avoid the predicted next behavior. For example, if the current spatial features of the array are such that the spatial features indicate that a tip fold over has occurred, a prediction can be made that continued insertion will further exasperate the tip fold over condition. In this instance, a plausible scenario is that the surgeon will not further insert the electrode array upon notification by the system that this is the case. But note that this prediction can also be utilized by the system to determine that another action should be taken. In this regard, the artificial intelligence subsystem could not only predict the next behavior, but also could determine what action should be taken based on that prediction. That is, the artificial intelligence subsystem could stand in the place of the surgeon or the healthcare professional. Note that in an alternate embodiment, it can be another artificial intelligence subsystem that takes the prediction and determines what is to be done in the next movement or otherwise determined whether the array is in a good position or not in a good position. That said, it need not be an artificial intelligence subsystem. In an exemplary embodiment, the system includes a number of preordained actions that should be taken based on various predictions and/or preordained relative locations of the array to the modiolus wall. For example, in the after mentioned example, if the prediction is further exasperation of the tip fold over, a lookup table can exist in the system where for that prediction, there is the action of withdrawing the electrode array by a certain amount, or based on a set of distances from the electrode array to the modiolus wall (the estimated modiolus wall), this set of distance can be found on the lookup table and the associated go no go data therefore can be identified. And note that this can be in terms of degrees, such as if the tip fold over condition is more severe than other conditions, more withdrawal of the array could be in order, and thus the lookup table could have different distances for withdrawal correlated to different tip fold over conditions, or the lookup table can have different degrees of acceptability such as, for example, if the operation has been difficult so far, it may not be desirable to try to reposition the electrode array whereas if the insertion process is gone relatively smoothly, it might be advisable to try to reposition the electrode array even though there are risks associated with doing such. And it is briefly noted that while this embodiment utilizes an artificial intelligence subsystem to make the prediction, in other embodiments, “big data” can be utilized to make the prediction. More on this below.

But note that instead of making a prediction and/or in addition to making a prediction, the system can determine the next movement of the array that should be taken based on the input. In a similar vein, instead of making a determination as to how close the array as to the modiolus wall (the estimated modiolus wall), the system can determine whether or not such is acceptable. In this regard, the system need not necessarily know or otherwise “understand” or make a determination of what will happen with respect to the next behavior of the array, or the ramifications of a given distance or how good or bad a given distance is or whether that distance can be improved. An artificial intelligence subsystem can be used to determine what should be done and/or whether or not such is acceptable. By rough analogy, a child may not know what will happen if he or she puts a key into the hot portion of an electrical outlet. Electricity and the concepts thereof may be completely alien and unknown to the child. However, the child can know not to put the key in the hot portion. Conversely, the child can know to put the key into a door to unlock the door.

With respect to the modiolus wall locations, again, a prediction can be made that further insertion of a certain amount or at least a certain amount will result in an increase in the average distance of the array from the wall. A prediction could be made that while there is currently a clinically significant separation of the array from the wall, continued further advancement at a given rate will eliminate that separation or potentially reduce that separation to an amount that is acceptable.

And in an exemplary embodiment, any one or more of the aforementioned movements could be the determination made with the use of the artificial intelligence subsystem corresponding to the above-noted next movement of the array that should be taken based on the input. And again, a prediction of what will result need not necessarily exist. The artificial intelligence subsystem can be configured with sufficient training or otherwise with sufficient data to “know” what to do based on the past history and/or the current spatial factors. This determination can be provided to the surgeon as a recommendation and/or can be presented as a control data set that can be approved or overridden by the surgeon as noted above.

In an embodiment, the artificial intelligence subsystem is a neural network that is a partially taught neural network that is trainable with feedback provided through the input suite or another subsystem of the system. Additional information about the training is discussed below.

In an embodiment, there is an apparatus, comprising a medical device insertion device, such as, by way of example, a cochlear implant electrode array insertion device. As mentioned above, this can be a robotic actuator such as an actuator of insertion device or, in some other embodiments as will be described in greater detail below, a more expanded version of a robot. That said, the device need not be a robot/have an actuator. Not all embodiments include an actuator that moves the array. The device can be a recommendation/physician's assistant device that instructs/recommends actions to take, where the surgeon inserts the array by hand or inserts the array using an automated device not in signal communication with the device.

In an exemplary embodiment, this insertion device includes an input component configured to receive data based on data related to an insertion process of a cochlear implant electrode array, the received data being received in real time relative to the process. Additional details of this will be discussed below, but note that the input component can correspond to any of that disclosed herein for receiving data based on data relating to the insertion process. By data based on data, this can correspond to the raw data, or a transposed version or manipulated version of the data, or new data that is developed based on the raw data. In an exemplary embodiment, this could be the output from a sensor, and thus the raw data from the sensor. In an embodiment, this can be data that corresponds to an evaluation of what that role sensor data means, and hence data based on data. The data could be from the imaging device, etc. (And the systems can include an integrated system that includes the insertion device and the imaging device (e.g., robot and the cone CT scan device)).

In an embodiment, the insertion device at least one of (i) includes non-transitory logic or (ii) has access to non-transitory logic and/or results of analysis of the non-transitory logic. The former is part of the device, wherein the latter is not part of the device. This latter arrangement could have utilitarian value with respect to having the logic or otherwise the “brains” in a remote location, where the device accesses that remote logic via the Internet or the like. This can enable the logic to be controlled and otherwise supervised and managed by a single entity, which entity could be unrelated to the entity utilizing the insertion device. This can have utilitarian value with respect to quality control and, in at least some exemplary embodiments, constantly updating or otherwise retraining the product of machine learning if such corresponds to the logic. Thus, in an exemplary embodiment, the logic can be artificial intelligence logic, such as, for example, the results of the product of machine learning.

In an embodiment, the non-transitory logic identifies the action to take (the “at least one of” where the device has the output component) and/or the locations and/or the adequacy of the positioning based on the received data, the received data being related to the insertion process including a prior action of the actuator if present (not all embodiments include an actuator that moves the array—again, the device can be a recommendation/physician's assistant device that instructs/recommends actions to take, where the surgeon inserts the array by hand or inserts the array using an automated device not in signal communication with the device) or another actuator (the another actuator could be separate from the device) configured to move the array in a manually controlled manner and/or a prior movement of the electrode array.

In an exemplary embodiment, the actions of evaluating/analyzing, etc., can be executed on raw data and/or modified data or filtered data or transformed data (hence analyzing data based on the obtained data—this can be the data or data that has been transformed). In an exemplary embodiment, the product of and/or the results of machine learning, or otherwise artificial intelligence, used to execute the analysis/evaluation, etc., and to otherwise make the predictions/determinations/evaluations/analyzes, etc., is a chip that is fabricated based on the results of machine learning. In an exemplary embodiment, the product/result is a neural network, such as a deep neural network (DNN). The product can be based on or be from a neural network. In an exemplary embodiment, the product/results is code. In an exemplary embodiment, the product/results is a logic circuit that is fabricated based on the results of machine learning. The product/results can be an ASIC (e.g., an artificial intelligence ASIC). The product/results can be implemented directly on a silicon structure or the like. Any device, system, and/or method that can enable the results of artificial intelligence to be utilized in accordance with the teachings detailed herein, such as in a hearing prosthesis or a component that is in communication with a hearing prosthesis, can be utilized in at least some exemplary embodiments. Indeed, as will be detailed below, in at least some exemplary embodiments, the teachings detailed herein utilize knowledge/information from an artificial intelligence system or otherwise from a machine learning system. Embodiments include a device and/or system to execute any one or more or all of the functionalities detailed herein that utilize the aforementioned chip or products to implement those functionalities.

In an embodiment, the systems and devices and/or methods herein use prediction and/or determination and/or analytical etc., models to execute one or more or all of the “analytical” actions/functionalities detailed herein, and the models can be results of machine learning/a product of machine learning (either directly or indirectly/based thereon), or a DNN, etc., that has the functionality thereof, and executes the functionality based on input thereto in view of its training. Again, the prediction model/determination model, etc., can be/or can be part of/can be established/non-transitorally based in computer chip (as opposed to a processor) or an electronic circuit. The models can be electronics that have the function thereof. Consistent with the well-known phenomenon of machine learning, it may not be that the models can be specifically understood. It may not be known exactly how the prediction model works, consistent with how artificial intelligence works in general and machine learning works specifically. The models can be the results of the machine learning as noted above. This can be the machine learning as frozen in time (when the learning was halted to establish the product). In an exemplary embodiment, an algorithm can include and/or the models herein can be based on a linear model, such as y=β₀+Σβᵢxᵢ+ε. Here, y is the outcome being predicted, β are coefficients and x are inputs.

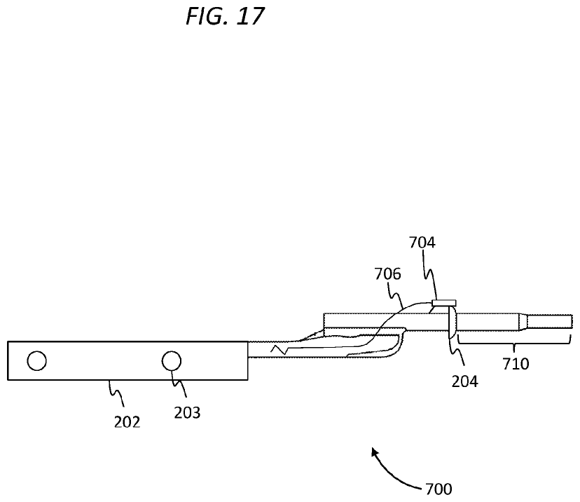

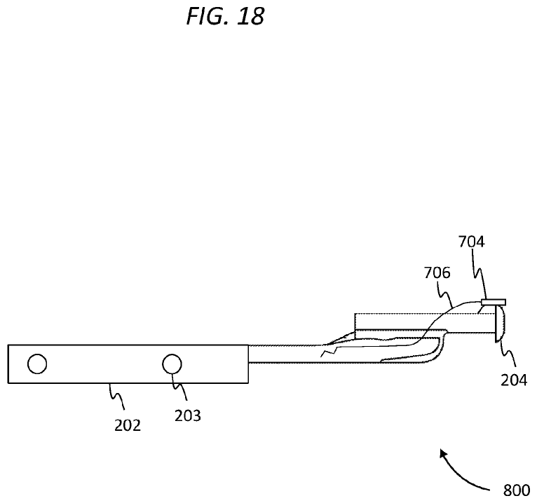

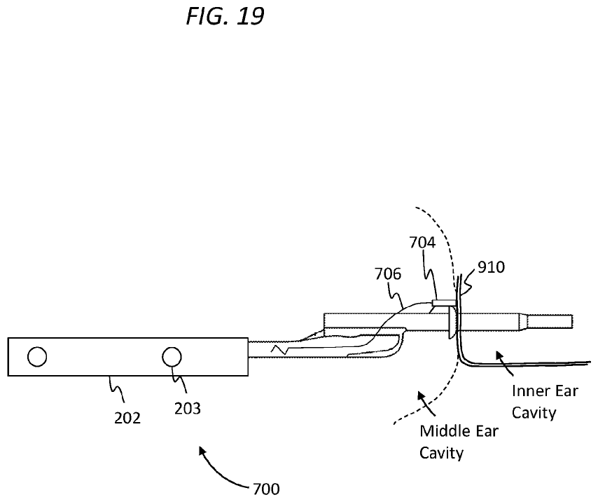

FIG. 17 depicts an exemplary embodiment of a cochlear electrode array insertion guide 700. In an exemplary embodiment, the insertion guide 700 corresponds to that of the insertion guide 200 detailed above, with the exception of the addition of electrode 704, and the modifications to the tool so as to support the electrode and the associated components thereof (e.g., electrical leads 706 (only the “distal” portion of the lead (distal relative to the tool 800) is depicted, the “break’ being conceptual), etc.—more on this below). Accordingly, FIG. 17 depicts a cochlear electrode array insertion guide comprising an array guide (e.g., the insertion guide tube (210 of FIG. 2)) and an active functional component (e.g., electrode 704). Some additional details of some exemplary functional components, including some exemplary active functional components, will be described in greater detail below. However, it is briefly noted at this time that not all embodiments of the cochlear electrode array insertion guide include an intracochlear portion. In this regard, FIG. 17 depicts a tool 700 that includes an intracochlear portion 710. This is the portion to the right of stop 204/the portion on the distal side of stop 204 (distal relative to the entire insertion guide). Conversely, FIG. 18 depicts a tool 800 that does not include an intracochlear portion. Instead, stop 204 is configured to be placed against the outside of the cochlea such that the passageway through the tool through which the electrode array is passed is aligned with the pertinent window and/or cochleostomy such that no parts of the tool 800 enters the cochlea.

It is noted that while the teachings detailed herein with respect to extra functionality of the insertion guide are based on the insertion guide detailed above with respect to FIGS. 5A-6B, these teachings can be applicable to other types of insertion guides. Indeed, as will be detailed below, some embodiments of the insertion guides do not have an intracochlear portion at all. Accordingly, the teachings above with respect to FIGS. 5A-6B serve as but one example of an insertion guide that the following teachings can be utilized in conjunction therewith.

With reference back to FIG. 17, the exemplary active functional component can be an electrode (read or energizing, etc.).

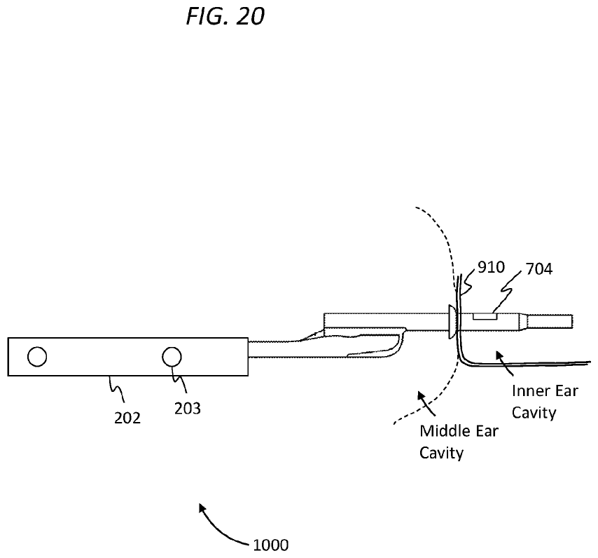

While the embodiments detailed above have focused on the electrode being located entirely outside the cochlea (e.g., entirely inside the middle ear), in an alternative embodiment, the electrode is located inside the cochlea during use. FIG. 20 depicts an exemplary insertion regime utilizing exemplary electrode array insertion guide 1000 where the electrode is located entirely in the inner cavity (in the cochlea) when the insertion guide is fully inserted into the inner ear cavity. Still further, FIG. 21 depicts an exemplary insertion regime utilizing exemplary electrode array insertion guide 1100 where the electrode being is located in the wall that separates the middle ear cavity from the inner ear cavity when the insertion guide is fully inserted into the inner ear cavity.

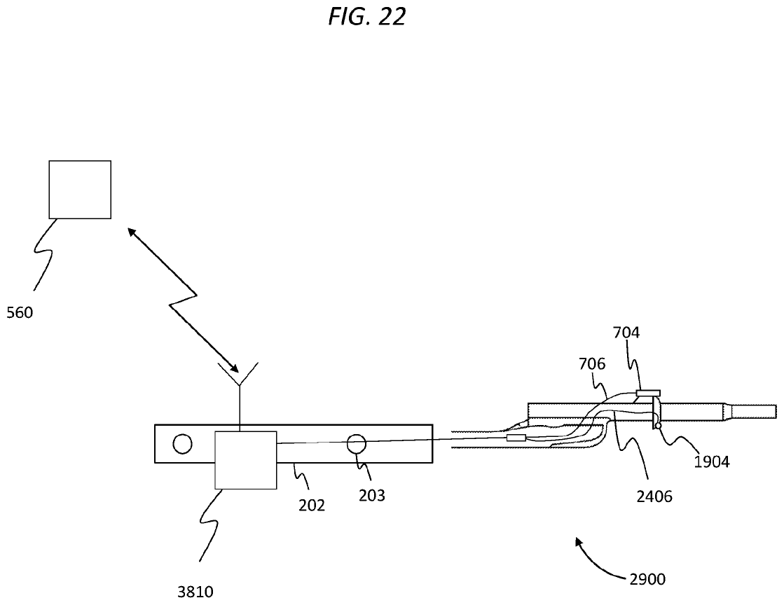

FIG. 22 depicts an insertion guide 2900 that is in wireless communication via element 3810 with a remote component 560, which could be a test unit or a control unit as disclosed further below.

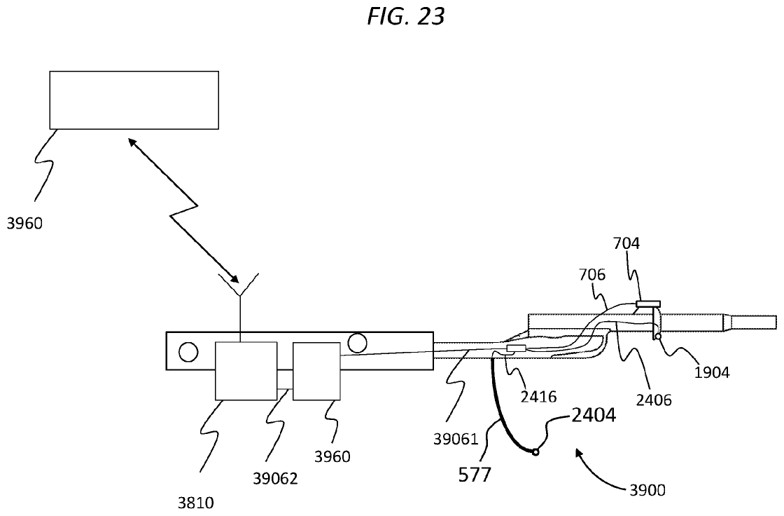

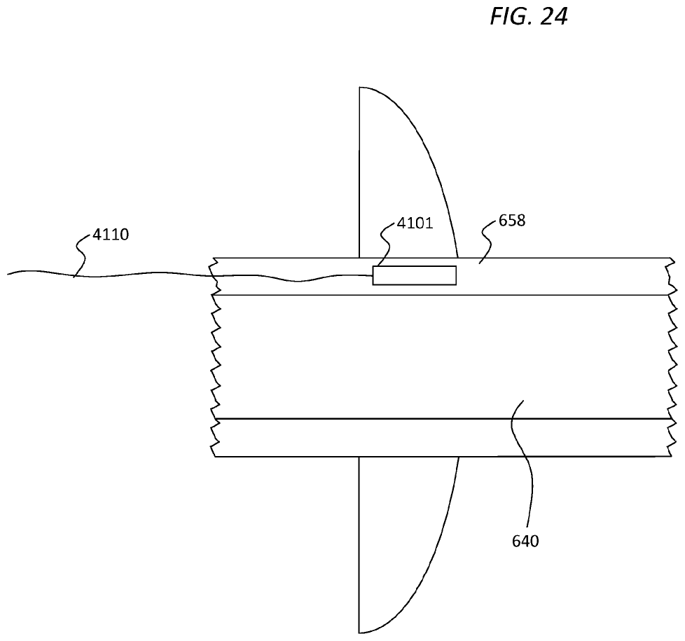

As briefly noted above, in at least some exemplary embodiments, some exemplary insertion guides can include a self-contained measurement system. FIG. 23 depicts such an exemplary embodiment of an insertion guide 3900. Insertion guide 3900 contains a complete measurement system. As can be seen, the insertion guide 3900 further includes a reference electrode 2404, which is in signal communication with the electrical leads of the system via lead 2416. Lead 39061 extends from the connector to test unit 3960, which can correspond to test a test unit configured to executes one or more or all of the test teachings herein, and can be a personal computer programmed to execute such. Test unit 3960 is in signal communication with communication unit 3810 via lead 39062. Communications unit 3810 can be in wireless communications with remote device 3960. In an exemplary embodiment, the remote device 3960 is a data storage device/data recording device that records the data transmitted via the communications unit 3810. For example, 3960 can be a desktop and/or a laptop computer having memory therein to record the data. In an alternate embodiment, device 3960 can be a control unit or the like, again such as a computer, that can control measurement system of the guide 3900. That said, in an exemplary embodiment, the guide 3900 includes an activation switch or the like so that the system can be activated and/or deactivated by the surgeon or other healthcare professional.

FIG. 24 depicts another exemplary embodiment of an insertion guide that has a functionality beyond that of an electrode array support/an electrode array insertion device. Particularly, the embodiment of FIG. 24 depicts a portion of the insertion guide tube at the stop 204 where a sensor 4101 is located in the wall 658 of the tube, although in other embodiments, the sensor 4101 is located on the inside wall of the tube and in other embodiments, the sensor 4101 is located on the outside wall of the tube. In this exemplary embodiment, the sensor is configured to sense or otherwise detect individual electrodes in the array as they pass by the sensor as the electrode array is inserted through the lumen 640 into the cochlea, and output a signal via lead 1410 indicative of at least one of an electrode passing the sensor 4101 or, in a more sophisticated embodiment, the speed of the electrode/electrode array passing by sensor 4101. In an exemplary embodiment, the sensor 4101 can be a sensor that utilizes capacitive sensing. In an exemplary embodiment, it could be a Hall effect sensor. In some embodiments, the sensor could be a sensor that comes into direct contact with the electrodes of the electrode array. In an exemplary embodiment, there is a system that receives the signal from lead 1410 and outputs data indicative of the insertion speed of the electrode. In an exemplary embodiment, the system can be a personal computer with an algorithm that analyzes the signal 4110, and outputs data to the surgeon. Exemplary output can be output by a speaker or the like indicating the speed of the insertion of the electrode array. (Feedback can be provided to a surgeon conducting a manual insertion such as by an audio system, or a visual system, the latter of which could be provided in a heads-up type display within the visual field of an operating microscope.)

Exemplary output can be output by a visual device indicating the speed of insertion of the electrode array. Exemplary output can correspond to the speed of insertion, a go/no go data package (e.g., insertion too fast/insertion speed fine). Such can be done via audio and/or visual devices. For example, a green light can indicate acceptable speed and a red light can indicate an unacceptable speed. Moreover, the system can be binary. The activation of the light will indicate that the speed is too fast/the audio indication (which could be a buzzer or a tone, etc.) activates when the insertion speed is too fast. The alternative could also be the case. The tone and/or light can be activated while the insertion speed is acceptable, and the tone or light is deactivated when the insertion speed is unacceptable. It will be noted that these indicators can also be utilized to indicate other sensed phenomenon or otherwise detected phenomenon as detailed herein.

In an exemplary embodiment, any of the teachings of U.S. Patent Application Publication No. 2018/0050196, to inventor Nicolas Pawsey, Published on Feb. 22, 2018, can be used to insert the array, and the teachings therein can be combined with the present teachings to implement the teachings herein.

Any arrangement of the insertion guide that can enable the teachings herein can be used in some embodiments.

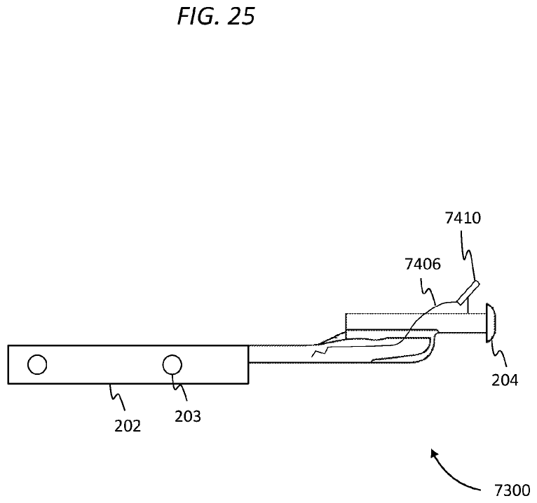

As noted above, the insertion guide can incorporate visual indicators to provide intraoperative feedback to the surgeon. As detailed above, exemplary embodiments have LEDs or the like arrayed about the stop. Still further, in an exemplary embodiment, a liquid crystal display or the like can be incorporated in or on the insertion guide. In this regard, FIG. 25 depicts an exemplary embodiment of an insertion guide 7300 which includes LCD 7410 mounted on the insertion guide tube. LCD 7410 is in electrical communication with other components of the guide and/or other systems remote from the guide via electrical lead 7406. In an exemplary embodiment, the LCD can provide text and/or numerical data to the surgeon during implantation/insertion of the electrode array. This can provide the instruction/recommendation as noted above to the surgeon. The LCD or the other visual indicators can be located anywhere on the guide that will be within the surgeon's immediate field-of-view, but also where the indicator will not obstruct the surgeon's field-of-view of the pertinent portions of the anatomy of the recipient and/or the pertinent portions of the guide 7300 during insertion of the electrode array. In an exemplary embodiment, the indicators provide information pertaining to insertion depth, which can include the absolute depth and/or an indication that the electrode array has reached the intended or programmed stopped depth. Indication can be an insertion speed, which can be absolute speed of insertion or can be an indication that the insertion speed limit has been exceeded. The indication can be an adverse measurement indication. This measurement can be a general indication, such as an indicator that something has gone wrong whatever that is, or specific indication, such as an indication explicitly relating to tip fold over, basilar membrane contact, scala dislocation, etc. Accordingly, in an exemplary embodiment, such indication can correspond to any of the anomalous electrode position indicators detailed herein.

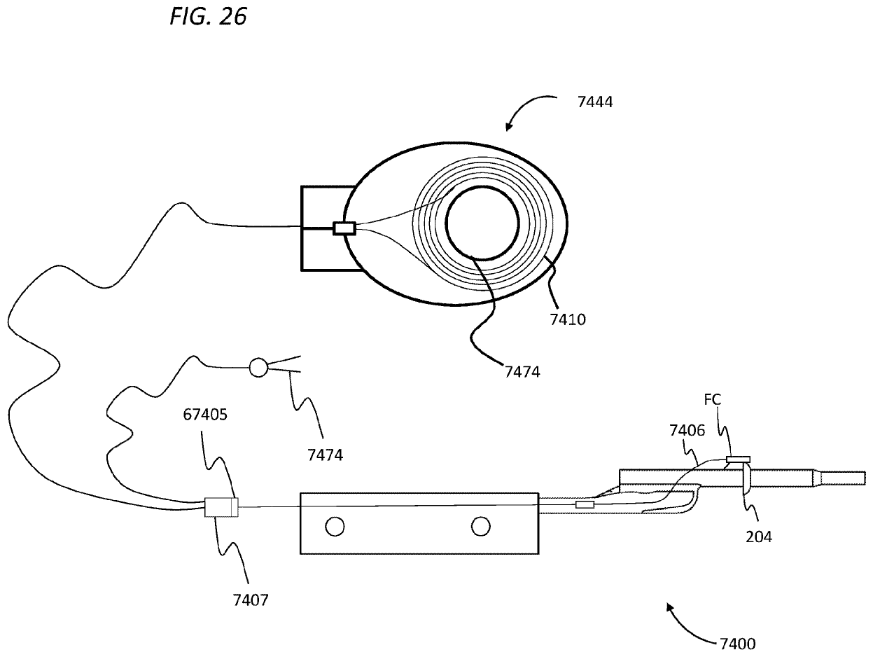

As noted above, embodiments include an insertion guide configured to communicate with a receiver/stimulator of a cochlear implant. In this regard, FIG. 26 depicts an exemplary insertion guide 7400 which is presented by way of concept. Insertion guide 7400 is a functional component FC mounted thereon. This functional component is representative of any of the additional functionalities of the insertion guide detailed herein and/or variations thereof. For example, element FC could be an electrode, it could be the acoustic stimulation generator, or it could be the ultrasonic transducer. FC could also be any of the indicators detailed herein (e.g., the LCD screen). As can be seen, insertion guide 7400 includes connector 64705 in electrical communication with the functional component FC via electrical lead 746. Connector 64705 is connected to connector 7407 of inductance coil 7444. In an exemplary embodiment, inductance coil 7444 includes coil 7410 configured to establish a magnetic inductance field so as to communicate with the corresponding coil of the receiver-stimulator of the cochlear implant. Inductance coil 7444 includes a magnet 7474 so as to hold the inductance coil 7474 against the coil of the receiver/stimulator of the cochlear implant in a manner analogous to how the external component of the cochlear implant is held against the implanted component, and how the coils of those respective components are aligned with one another. While the embodiment depicted in FIG. 26 depicts no other functional component between the functional component FC and the inductance coil 7444, in an alternate embodiment, one or more of the units detailed herein can be located there between. By way of example, generator 6520 with respect to the insertion guide 6500 detailed above can be located therebetween or otherwise be in signal communication with the leads so as to establish communication with that element with the cochlear implant. In an exemplary embodiment, a communications unit or the like is located between or otherwise is in signal communication with the leads so as to establish communication with the cochlear implant receiver-stimulator. In an exemplary embodiment, the insertion guide includes logic or a processor or other type of control unit that enables the insertion guide to work in conjunction with the cochlear implant so as to execute any of the methods detailed herein, such as, for example, where one or more electrodes of the electrode array insertion guide are utilized in a state of one or more electrodes of the electrode array as taught in those applications.

FIG. 26 also shows second lead from connector 7407 extending to alligator clip 7474, which in an exemplary embodiment, configured to clip onto the hard ball and/or the can of the implant, in which clip is in electrical communication with one or more electrodes on the electrode array that would be inside and/or outside of the cochlea during insertion. Indeed, it is also noted that in an exemplary embodiment, the entire portion that is inserted into the cochlea of the insertion guide can be the electrode, and thus be in electrical communication with the alligator clip 7474.

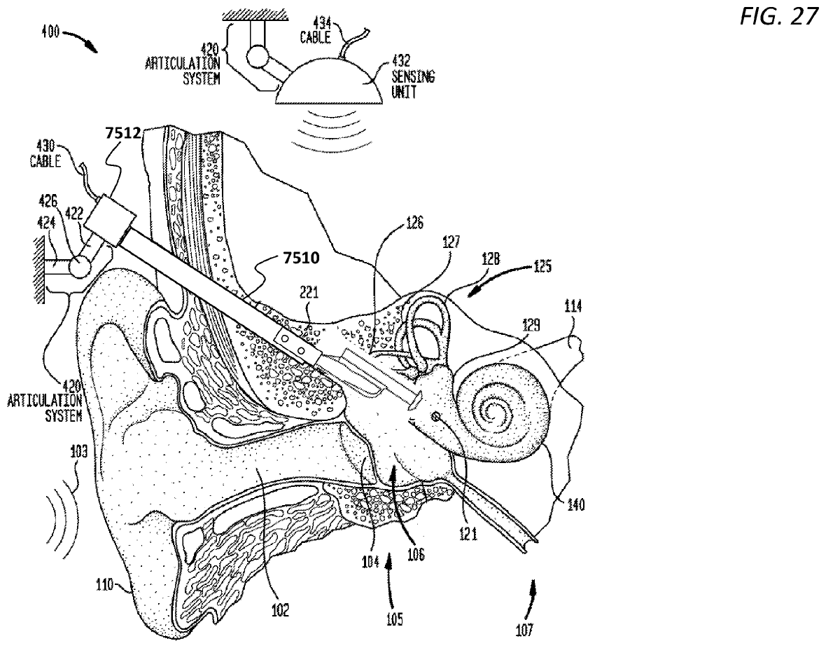

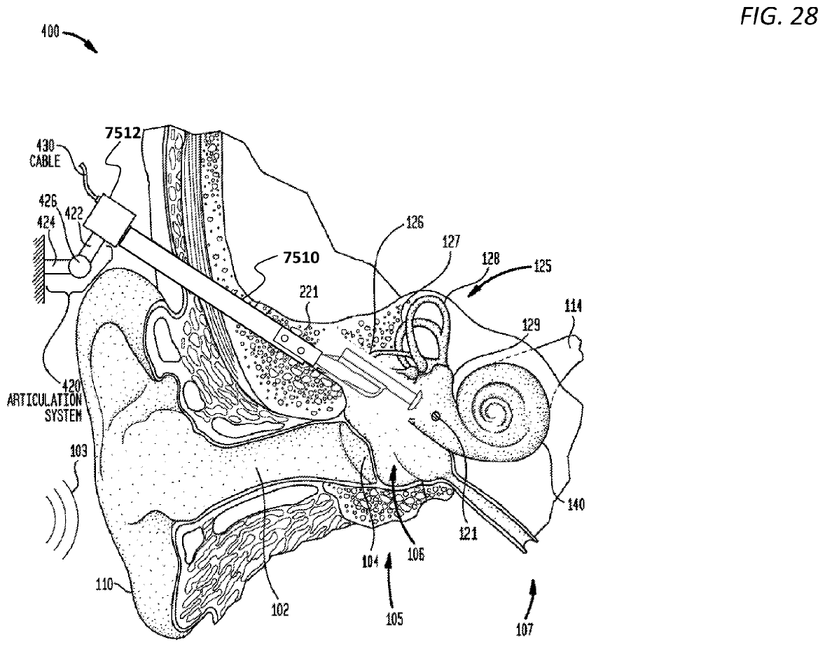

It is noted that at least some exemplary embodiments include utilization of the insertion guides detailed herein and/or variations thereof with a robotic electrode array insertion system. In this regard, FIG. 27 is a perspective view of an exemplary embodiment of an insertion system 400. It is noted that the embodiment depicted in FIG. 27 is presented for conceptual purposes only. Features are provided typically in the singular show as to demonstrate the concept associated therewith. However, it is noted that in some exemplary embodiments, some of these features are duplicated, triplicated, quadplicated, etc. so as to enable the teachings detailed herein and/or variations thereof. Briefly, it is noted that any teaching detailed herein can be combined with a robotic apparatus and/or a robotic system according to the teachings detailed herein and/or variations thereof. In this regard, any method action detailed herein corresponds to a disclosure of a method action executed by a robotic apparatus and/or utilizing a robot to execute that action and/or executing that method action is part of a method where other actions are executed by robot and/or a robotic system etc. Still further, it is noted that any apparatus detailed herein can be utilized in conjunction with a robotic apparatus and/or a robot and/or a system utilizing such. Accordingly, any disclosure herein of an apparatus corresponds to a disclosure of an apparatus that is part of a robotic apparatus and/or a robotic system etc. and/or a system that includes a robotic apparatus etc.

System 400 includes a robotic insertion apparatus including arm 7510 to which insertion guide 200 or any other insertion guide according to the teachings detailed herein and/or variations thereof is attached (e.g., bolted to arm 7510). In this exemplary embodiment, arm 7510 is depicted as a single structure extending from the insertion guide to mount 7512. However, in an alternate embodiment, arm 7510 can be a multifaceted component which is configured to articulate at various locations thereabout.

In an exemplary embodiment, arm 7510 is releasably connected by way of a releasable connection to mount 7512, which is supported by a support and movement system 420, comprising support arm 422 which is connected to joint 426 which in turn is connected to support arm 424. Support arm 424 is rigidly mounted to a wall, a floor, or some other relatively stationary surface. That said, in an alternative embodiment, support arm 424 is mounted to a frame that is attached to the head of the recipient or otherwise connected to the head of the recipient such that global movement of the head will result in no relative movement of the system 400 in general, and the insertion guide in particular, relative to the cochlea. Joint 426 permits arm 2510, and thus the insertion guide, to be moved in one, two, three, four, five, or six degrees of freedom. (It is noted again that FIG. 27 is but a conceptual FIG.—there can be joints located along the length of arm 7510, so as to enable arm 75102 articulate in the one or more of the aforementioned degrees of freedom at those locations. In an exemplary embodiment, joint 426 includes actuators that move mount 7512, and thus the insertion guide, in an automated manner, as will be described below. In an exemplary embodiment, the system is configured to be remotely controlled via communication with a remote control unit via communication lines of cable 430. In an exemplary embodiment, the system is configured to be automatically controlled via a control unit that is part of the system 400. Additional details of this will be described below.

The system 400 further includes by way of example only and not by way of limitation, sensor/sensing unit 432. That said, in some embodiments, sensor 432 is not part of system 400. In some embodiments, it is a separate system. Still further, in some embodiments, it is not utilized at all with system 400. While sensor 432 is depicted as being co-located simultaneously with the insertion guide, etc., as detailed below, sensor 432 may be used relatively much prior to use of the insertion guide. Sensing unit 432 is configured to scan the head of a recipient and obtain data indicative of spatial locations of internal organs (e.g., mastoid bone 221, middle ear cavity 423 and/or ossicles 106, etc.) In an exemplary embodiment, sensing unit 432 is a unit that is also configured to obtain data indicative of spatial locations of at least some components of the insertion guide and/or other components of the robotic apparatus attached thereto. The obtained data may be communicated to remote control unit 440 via communication lines of cable 434. As may be seen, sensor 432 is mounted to a support and movement system 420 that may be similar to or the same as that used by the robotic apparatus supporting the insertion guide.

In an exemplary embodiment, sensing unit 432 is an MRI system, an X-Ray system, an ultrasound system, a CAT scan system, or any other system which will permit the data indicative of the spatial locations to be determined as detailed herein and/or variations thereof. As will be described below, this data may be obtained prior to surgery and/or during surgery. It is noted that in some embodiments, at least some portions of the insertion guide are configured to be better imaged or otherwise detected by sensing unit 432. In an exemplary embodiment, the tip of the insertion guide includes radio-opaque contrast material. The stop of the insertion guide can also include such radio-opaque contrast material. In an exemplary embodiment, at least some portions of insertion guide in general, and the robotic system in particular, or at least the arm 7510, mount 7512, arm 422, etc., are made of non-ferromagnetic material or other materials that are more compatible with an MRI system or another sensing unit utilized with the embodiment of FIG. 27 than ferromagnetic material or the like. As will be described in greater detail below, the data obtained by sensing unit 432 is used to construct a 3D or 4D model of the recipient's head and/or specific organs of the recipient's head (e.g., temporal bone) and/or portions of the robotic apparatus of which the insertion guide is a part. That said, to be clear, in some embodiments, sensing unit 432 is not present, as seen in FIG. 28. Noter that these imaging/sensing systems can be used to determine one or more of the spatial features detailed herein.

It is also noted that in some exemplary embodiments of system 400, there are actuators or the like that drive the electrode array through the insertion guide into the cochlea. These actuators can be in signal communication with the control unit. In an exemplary embodiment, the control unit can control the actuators to push the electrode array into and/or out of the cochlea as will be described in greater detail below. Concomitant with the robotic assembly supporting the insertion guide, in an exemplary embodiment, the control unit is configured to automatically control these actuators.

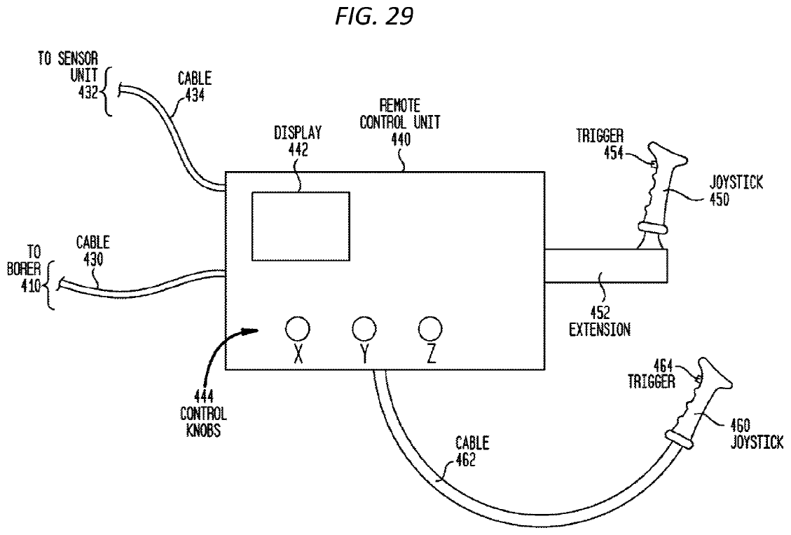

FIG. 29 is a simplified block diagram of an exemplary embodiment of a remote control unit 440 for controlling the robotic apparatus supporting the insertion guide and sensing unit 432 via communication lines 430 and 434, respectively. Again, it is noted that in some alternate embodiments, the remote control unit 440 is an entirely automated unit. That said, in some alternate embodiments, the remote control unit can be operated automatically as well as manually, which details will be described below.

Remote control unit 440 includes a display 442 that displays a virtual image of the mastoid bone obtained from sensor 432 and may superimpose a virtual image of the insertion apparatus onto the virtual image indicative of a current position of the drill bit relative to the ear anatomy. An operator (e.g., surgeon, certified healthcare provider, etc.) utilizes remote control unit 440 to control some or all aspects of the robotic apparatus and/or sensing unit 432. Exemplary control may include depth of insertion guide insertion, angle of guide insertion, speed of advancement and/or retraction of electrode array, etc. Such control may be exercised via joystick 450 mounted on extension 452 which fixedly mounts joystick 450 to a control unit housing. Such control may be further exercised via joystick 460 which is not rigidly connected to housing of remote control unit 440. Instead, it is freely movable relative thereto and is in communication with the remote control unit via communication lines of cable 462. Joystick 462 may be part of a virtual system in which the remote control unit 440 extrapolates control commands based on how the joystick 462 is moved in space, or joystick may be a device that permits the operator more limited control over the cavity borer 410. Such control may include, for example an emergency stop upon release of trigger 464 and/or directing the robot to drive the insertion guide further into the cochlea by squeezing the trigger 464 (which, in some embodiments, may control a speed at which the insertion guide is advanced by squeezing harder and/or more on the trigger). In the same vein, trigger 454 of joystick 450 may have similar and/or the same functionality.

Control of the robot assembly supporting the insertion guide may also be exercised via knobs 440 which may be used to adjust an angle of the insertion guide in the X, Y and Z axis, respectively. Other controls components may be included in remote control 440.

FIG. 29 depicts an exemplary insertion guide which can correspond to any of the insertion guide detailed herein and/or variations thereof, or any other insertion guide for that matter, further including an electrode array insertion actuator 7720. In an exemplary embodiment, actuator assembly 7720 includes a passageway therethrough through which the electrode array extends. The actuator assembly drives the electrode array in a manner replicating that by which the surgeon pushes the electrode array forward along the insertion guide and into the insertion tube and thus into the cochlea.

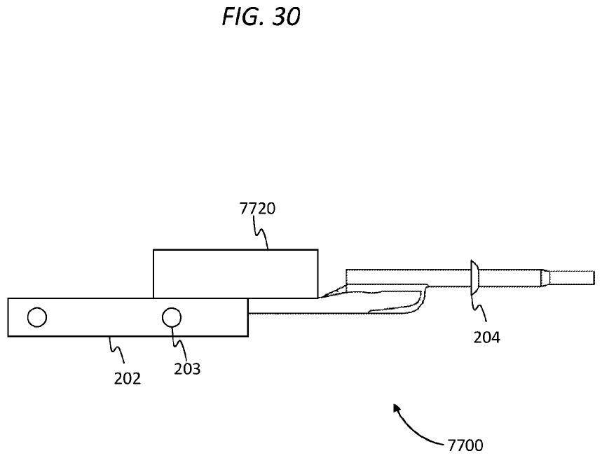

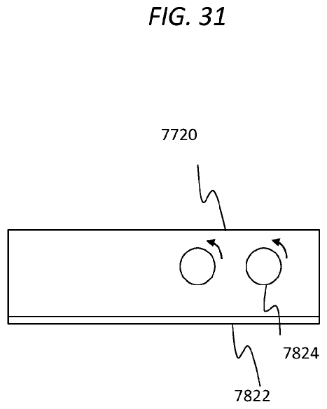

FIG. 30 depicts an exemplary embodiment of the actuator assembly 7720. As can be seen, actuator assembly includes two actuators 7824 in the form of wheels mounted to electric motors that rotate the wheels in a counterclockwise direction so as to advance the electrode array, and in a clockwise direction so as to retract the electrode array. Actuator assembly 7720 further includes a floor 7822. The floor 7822 works in combination with the actuators 7824 so as to “trap” the electrode array there between with a sufficiently compressive force so that the friction forces between the actuators 7824 and the electrode array enable the actuators 7824 to drive the electrode array forward and/or backwards, but not enough so as to damage the electrode array. FIG. 31 depicts an exemplary movement of the wheels 7824.

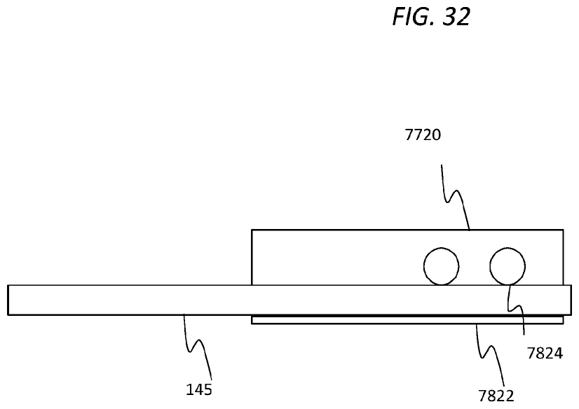

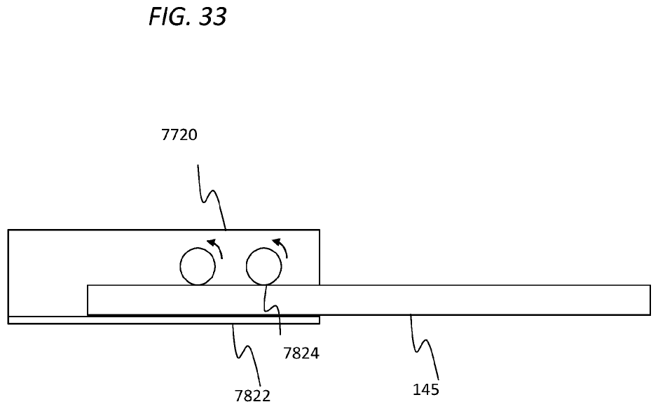

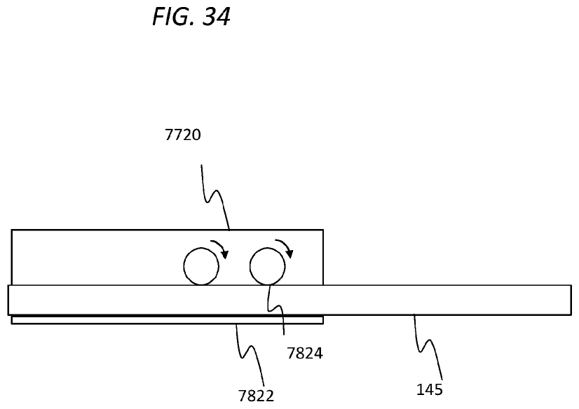

FIG. 32 functionally depicts an electrode array 145 “loaded” in actuator assembly 7720 prior to driving the electrode array into the insertion sheath. FIG. 33 functionally depicts the electrode array being driven forward (FIG. 33 is depicted in a functional manner—in reality, the electrode array 145 would extend up the ramp and then into the insertion sheath), and FIG. 34 functionally depicts the electrode array being retracted from the position seen in FIG. 33.

While the embodiment of the actuator assembly depicted in FIG. 32 includes two top actuators, in an alternate embodiment, only one top actuator is utilized and/or in another embodiment, three or four or five or six or more actuators are utilized. Also, in an exemplary embodiment, one or more bottom actuators can also be utilized. Note also that instead of the actuators being located on the top and the floor 7822 being on the bottom, the actuators can be located on the bottom and the floor can be located on the top.

It is noted that while the embodiment of FIG. 32 is depicted utilizing actuators having round wheels, in an alternate embodiment, other types of working and of the actuators can be utilized.

To be clear, the embodiment of FIG. 29 depicted above can also include the actuator assembly's detailed herein and/or variations thereof. That is, insertion guide 7700 can be attached to the arm 7510 of the system 400. Moreover, the actuators of the actuator assembly can be placed into signal communication with the control unit 440 or any other control unit of the system 400 to enable the control unit to advance and/or retract the electrode array. Note also that in some alternate embodiments, the system 400 is such that the only non-manually actuating component is the actuator assembly. That is, in an exemplary embodiment, system 400 can be such that the frame of the like is placed around the recipient's head and secured thereto, and the arm 7510 supporting the insertion guide attached thereto can be moved manually by the surgeon, such that the surgeon can align or otherwise place the insertion guide into the cochlea. In this regard, by way of example only and not by way of limitation, the insertion guide can be configured so as to attached to the arm 7510 on a trolley or the like. In an exemplary embodiment, the surgeon moves arm 7510 into position so that the insertion guide is aligned with the cochlea, at the desired angle, etc., and then be surgeon manually pushes the insertion guide forward into the cochlea (in the case of an intra-cochlear insertion guide) or against the cochlea in the case of a non-intra-cochlea insertion guide). After that, the actuator assembly can be utilized in a remote-controlled and/or automated manner.

That said, in an alternate embodiment, the general positions of the system 400 can be established utilizing manual methods, and then the positions can be refined utilizing automated/remote controlled methods (e.g., the actuators on the arm 7510 and/or the actuator at joint 426 can be actuated so as to finally position the insertion guide.

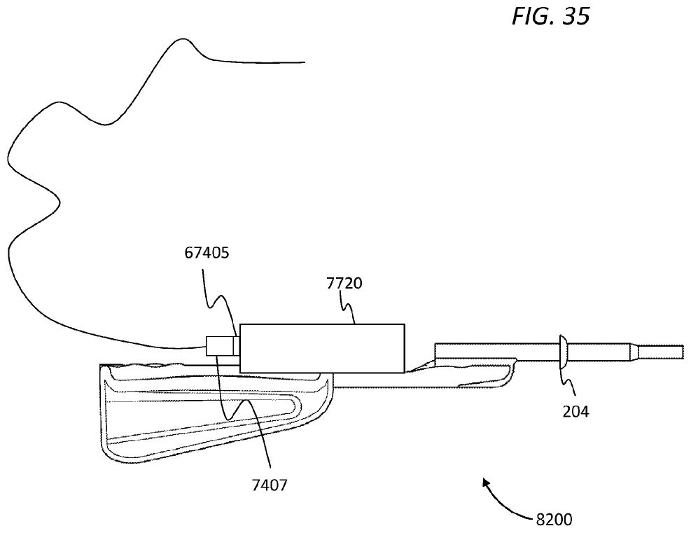

Note also that in some exemplary embodiments, the actuator assembly's detailed herein and/or variations thereof that are utilized to advance and/or retract the electrode array are configured to be utilized with an insertion tool that is handheld instead of being attached to arm 750 system 400. To this end, FIG. 35 depicts an exemplary insertion tool 8200 that includes actuator apparatus 7720 as seen. Hereinafter, the reference will often be made to actuator apparatus 7720 as utilized in conjunction with other components detailed herein. Any disclosure herein of the utilization of actuator apparatus 7720 in conjunction with other teachings detailed herein corresponds to a disclosure of the utilization of the actuator apparatus 8123 or any of the other actuator apparatuses detailed herein or variations thereof utilized to grip and support and/or insert the electrode array into the cochlea. FIG. 35 depicts a connector 67405 in signal communication with an actuator apparatus 7720, which connector is connected to connector 7407, which in turn is connected to a lead which extends to the control unit. In an exemplary embodiment, the surgeon holds the tool 8200 in the traditional manner of use, but the control unit controls the actuation of the actuator 7720 to advance and/or retract the electrode array. In an exemplary embodiment, the surgeon or other healthcare professional can exercise override control over the insertion of the electrode array and/or the retraction of the electrode array. For example, switching components of the like or other types of input devices can be located on the tool 8200 so that the surgeon or the like can provide input into the system of which the tool 8200 is a part. In an alternate embodiment, the tool 8200 can include an input device that interacts with the surgeon, where the surgeon provides the direction to the system advance and/or retract the electrode array, but the control unit evaluates the inputs from the surgeon and controls the actuation accordingly. By way of example only and not by way of limitation, such a system can be analogous to a fly by wire system on an aircraft, where the pilot moves the controls in a manner correlated to the direction that the pilot wants the aircraft to move, and the flight control system controls everything else to achieve the desired outcome. Note also that any the other actuators detailed herein and/or variations thereof can be part of a system that is operated in a similar manner. By way of example only and not by way of limitation, the system 400 can be configured such that the surgeon pushes on the arm 7510 to move the insertion guide is desired, but the system 400 moves the arm 7510 using actuators. That is, the system 400 is configured to sense or otherwise detect the force is applied on to the structure thereof by the surgeon, and then determine what actuator action should be executed so as to position the insertion guide at the desired location in a manner analogous to fly by wire.

It is noted that the electrical lead assembly and the connectors thereof depicted in FIG. 35 can be applicable to any of the insertion guides detailed herein and/or variations thereof so as to place the insertion guide in general, and the actuator assembly thereof in particular, into signal communication with the control unit or other controllers of the system. Note also that in an exemplary embodiment, the lead apparatus depicted in FIG. 35 can be utilized to also convey the other signals detailed herein and/or variations thereof with respect to the other functionalities associated with the insertion guides. Alternatively, and/or in addition to this, the other lead apparatuses detailed herein and variations thereof can be utilized to convey the signals from the actuator apparatus 7720 to the control unit or the like when the insertion guides detailed above are utilized in conjunction with the actuator assembly so as to provide a machine drive to advance and/or retract the electrode array. Any device, system and/or method of communication between any functional component of any of the insertion guides detailed herein and/or variations thereof with a control unit and/or vice versa and/or the implantable component of the electrode array, etc., can be utilized in at least some exemplary embodiments.

It is also noted that while the embodiments detailed herein have been directed towards an electrode array guide, it is also noted that in some alternate embodiments, an electrode array support is instead utilized, which support may not necessarily guide the electrode array, but otherwise might simply support the electrode array proximate to the cochlea. Note that in an electrode array support can also be an electrode array guide, and vice versa.

In view of the above, it can be understood that in an exemplary embodiment, there is an apparatus, such as any of the insertion guides detailed herein and/or variations thereof, that includes an electrode array support, and an actuator. In at least some of these exemplary embodiments, the apparatus is configured to inserts an electrode array into cochlea by a controlled actuation of the actuator. In an exemplary embodiment of such an exemplary embodiment, the controlled actuation is at least partially based on electrical phenomenon of the recipient. Some additional details of such will now be described.

Embodiments of the actuators and/or the insertion devices/robots herein, such as those shown in the figures just described, and/or the figures below, can be utilized to gather data regarding the spatial features that are utilized in the method systems and/or devices disclosed herein. In an exemplary embodiment, the detailed rollers, or more specifically, the movement of the rollers, can be utilized to gauge the distance of insertion of the electrode array. The idea being is that the outer circumference of the rollers will be known, and thus the angular rotation of the rollers can be correlated to movements of the array if there is no slippage between the rollers and the array. In this regard, the systems and devices can be configured so that the angular rotation can be determined, and this can be provided to the overall system to deduce the distance that the array has been inserted or withdrawn. But again, the other types of sensors can also be utilized. And note that while some embodiments have been described in terms of a device that has an automated insertion/retraction system, in other embodiments, the insertion devices do not have such, and instead are simply utilizes a guide or the like, where the surgeon's hand is utilized to apply the insertion and/or removal force. Any of the robotic devices disclosed herein can correspond to the robotic devices or otherwise the actuators described above with respect to the various embodiments. In some embodiments, the surgeon or other healthcare professional utilizes some of the robotic devices, while in other embodiments, the overall system controls these robotic devices.

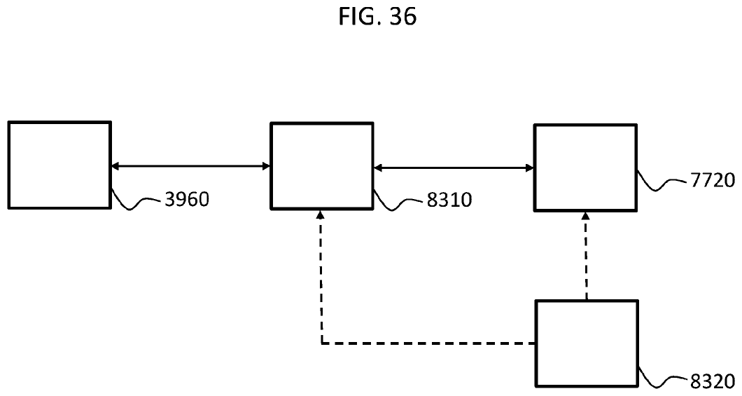

FIG. 36 depicts an exemplary functional schematic of an exemplary system that includes the test unit 3960 detailed above in signal communication with a control unit 8310 which is in turn in signal communication with the actuator assembly 7720. The test unit and the control unit can be one and the same in some embodiments.

It is also noted that in some embodiments, there is no control unit and/or there is no actuator assembly. That is, the system can be a purely test system, which conveys information to the surgeon or other healthcare professional to instruct (e.g., the output of the control unit and/or the test unit can be instead an instruction as opposed to a control signal) or otherwise provide an indication of the phenomenon to the surgeon or other healthcare professional.

Also functionally depicted in FIG. 36 is the optional embodiment where an input device 8320 is included in the system (e.g., which could be on an embodiment where the actuator assembly 7720 is part of a hand tool or where actuator assembly 7720 is part of an insertion guide, where the input device 8320 is located remote from the insertion guide, which could be part of a remote unit 440). In an exemplary embodiment, the input device 8320 could be the trigger for 54 and/or 464 of the remote control unit 440. In an exemplary embodiment, the input device 8320 could be a trigger on the tool 8200. Again, in an exemplary embodiment, the input device 8320 can be utilized to enable advancement and/or withdrawal of the electrode array, and the system 400 could control the advancement and/or withdrawal based on an automated protocol or some other flyby wire type system. In the embodiment of FIG. 36, the input device 8320 can be in signal communication directly to the actuator assembly 7720, and/or in signal communication with the control unit 8310.

In an exemplary embodiment, control unit 8310 can correspond to the remote unit 440. That said, in an alternate embodiment, remote unit 440 can be a device that is in signal communication with control unit 8310. Indeed, in an exemplary embodiment, input device 8320 can correspond to remote control unit 440.

More particularly, control unit 8310 can be a signal processor or the like or a personal computer or the like or a mainframe computer or the like etc., that is configured to receive signals from the test unit 3960 and analyze those signals to evaluate an insertion status of the electrode array. More particularly, the control unit 8310 can be configured with software the like to analyze the signals from test unit 3960 in real time and/or in near real time as the electrode array is being advanced into the cochlea by actuator assembly 7720. The control unit 8310 analyzes the input from test unit 3960 as the electrode array advanced by the actuator assembly 7720 and evaluates the input to determine if there exists an undesirable insertion status of the electrode array and/or evaluates the input to determine if the input indicates that a scenario could occur or otherwise there exists data in the input that indicates that a scenario is more likely to occur relative to other instances where the insertion status of the electrode array will become undesirable if the electrode array is continued to be advanced into the cochlea, all other things remaining the same (e.g., insertion angle/trajectory, etc., which can be automatically changed as well via—more on this below). In an exemplary embodiment, upon such a determination, control unit 8310 could halt the advancement of the array into the cochlea by stopping the actuator(s) of actuator assembly 7720 and/or could slow the actuator(s) so as to slow rate of advancement of the electrode array into the cochlea and/or could reverse the actuator(s) so as to reverse or otherwise retract the electrode array within the cochlea (either partially or fully). In at least some exemplary embodiments, control unit 8310 can be configured to override the input from input unit 8320 input by the surgeon or the user or the like of the systems herein.

In an exemplary embodiment, the outputs of test unit 3960 corresponds to the outputs indicated herein. Alternatively, and/or in addition to this, input into control unit 8310 can flow from other sources. Any input relating to the measurement of voltage associated executing the teachings herein into control unit 8310 can be utilized in at least some exemplary embodiments.

In an exemplary embodiment, control unit 8310 can be configured to determine, based on the input from test unit 3960, whether the electrode array has come into contact with the basilar membrane of the cochlea and/or that one or more of the anomalous electrode positions has occurred and/or whether there exists an increased likelihood that such will occur, and automatically control the actuator assembly 7720 accordingly. In an exemplary embodiment, control unit 8310 does not necessarily determine that such an insertion status exists or is more likely to exist, but instead is programmed or otherwise configured so as to control the actuator assembly 7720 according to a predetermined regime based on the input from the test unit 3960. That is, the control unit 8310 need not necessarily “understand” otherwise “know” the actual insertion status or the forecasted insertion status of the electrode array, but instead need only be able to control the actuator assembly 7720 based on the input.

In an exemplary embodiment, control unit 8310 can be configured to determine, based on the input from test unit 3960, the insertion depth of the electrode array and/or a forecasted insertion depth of the electrode array, and automatically control the actuator assembly 7720 accordingly. In an exemplary embodiment, control unit 8310 does not necessarily determine the insertion depth or forecasted insertion depth, but instead is programmed or otherwise configured so as to control the actuator assembly 7720 according to a predetermined regime based on the input from the test unit 3960. That is, the control unit 8310 need not necessarily “understand” otherwise “know” the actual insertion depth or the forecasted insertion depth of the electrode array, but instead need only be able to control the actuator assembly 7720 based on the input.

In an exemplary embodiment, control unit 8310 can be configured to determine, based on the input from test unit 3960, executing, for example, the methods/techniques disclosed herein, whether the electrode array has buckled and/or bent and/or any other anomalous electrode location as disclosed herein or otherwise may be the case and/or whether there exists an increased likelihood that such will occur, and automatically control the actuator assembly 7720 accordingly. In an exemplary embodiment, control unit 8310 does not necessarily determine that such buckling and/or bending exists or is more likely to exist, but instead is programmed or otherwise configured so as to control the actuator assembly 7720 according to a predetermined regime based on the input from the test unit 3960. That is, the control unit 8310 need not necessarily “understand” otherwise “know” that the electrode array has actually buckled or will buckle in the future, but instead need only be able to control the actuator assembly 7720 based on the input.

Thus, it can be understood that there is an apparatus that is configured to receive input indicative of the electrical phenomenon/phenomena inside the recipient, and develop data indicative of a position of the electrode array within the cochlea based on the input. (It is briefly noted that unless otherwise specified, the singular term phenomenon also includes a disclosure of the plural thereof, and vis-a-versa, as is also the case with the disclosure of data). Still further, such an exemplary embodiment can be configured to adjust the control of the actuation of the actuator based on the developed data indicative of the position of the electrode array.

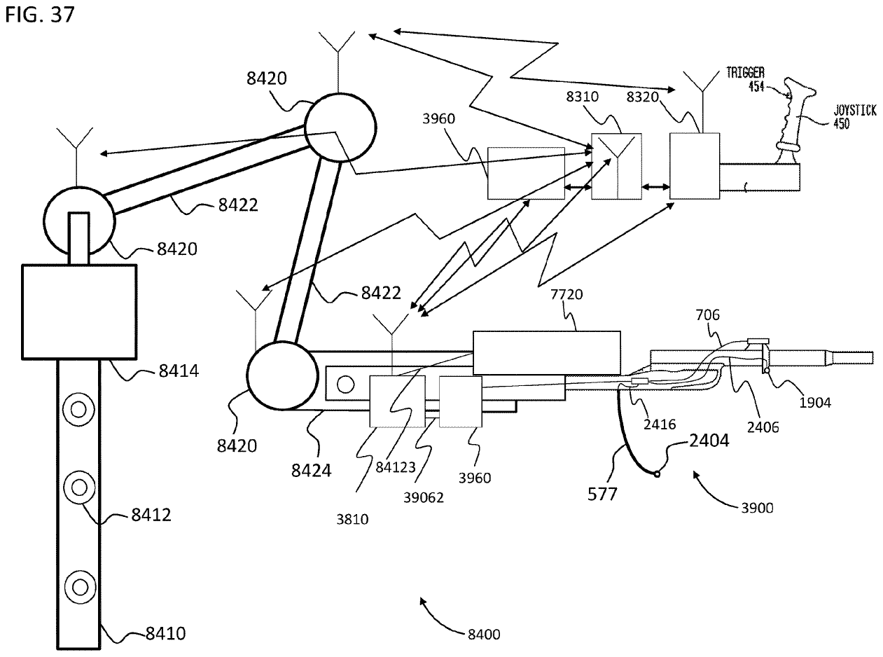

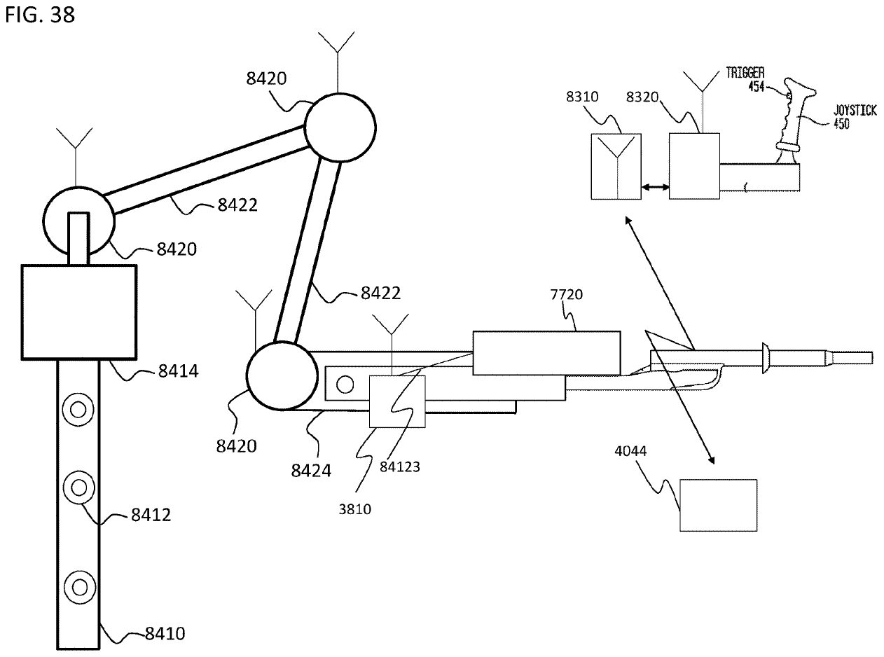

To be clear, while the embodiment detailed above is focused on controlling the actuator assembly 7720 based on data from the system so as to control the advancement and/or retraction of the electrode array based on the data disclosed herein and, in an alternate embodiment, the system 400 controls one or more other actuators of the robot apparatus of system 400. These one or more other actuators can be exclusive from the actuator assembly 7720, or can include the actuator assembly 7720. In this regard, FIG. 37 depicts an exemplary robot apparatus 8400, that includes the insertion guide 3900 detailed above with respect to the integration of a system ad disclosed herein therewith mounted on arm 8424 utilizing bolts in a manner concomitant with that detailed above. In an exemplary embodiment, robot apparatus 8400 has the functionality or otherwise corresponds to that of the embodiment of FIG. 29. In this regard, any functionality associated or otherwise described with respect to the embodiment of FIG. 29 corresponds to that of the embodiment of FIG. 37, and vice versa. In this exemplary embodiment, the actuator apparatus 7720 is in signal communication with unit 3810 via electrical lead 84123. In this regard, signals to and/or from the actuator assembly 7720 can be transmitted to/from the antenna of unit 8310 (in FIG. 38, the “Y” shaped elements are antennas) and thus communicated via lead 84123. It is briefly noted that while the embodiment depicted in FIG. 37 utilizes radiofrequency communication, in alternate embodiments, the communications can be wired. In an exemplary embodiment both can be utilized.

The robot apparatus 8400 includes a recipient interface 8410 which entails an arch or halo like structure made out of metal or the like that extends about the recipient's cranium or other parts of the body. The interface 8410 is bolted to the recipient's head via bolts 8412. That said, in alternate embodiments, other regimes of attachment can be utilized, such as by way of example only and not by way of limitation, strapping the robot to the recipient's head. In this regard, the body and interface 8410 can be a flexible strapping can be tightened about the recipient's head.

Housing 8414 is located on top of the interface 8410, as can be seen. In an exemplary embodiment, housing 8414 includes a battery or the like or otherwise provides an interface to a commercial/utility power supply so as to power the robot apparatus. Still further, in an exemplary embodiment, housing 8414 can include hydraulic components/connectors to the extent that the actuators herein utilize hydraulics as opposed to and/or in addition to electrical motors. Mounted on housing 8414 is the first actuator 8420, to which arm 8422 is connected in an exemplary embodiment, actuator 8420 enables the components “downstream” (i.e., the arm connected to the actuator, and the other components to the insertion guide) to articulate in one, two, three, four, five or six degrees of freedom. A second actuator 8420 is attached to the opposite end of the arm 8422, to which is attached a second arm 8422, to which is attached a third actuator 8420, to which is attached to the insertion guide attachment structure 8424. Elements 8422 and 8424 can be metal beams, such as I beams or C beams or box beams, etc. actuators 8420 can be electrical actuators and/or hydraulic actuators.

As can be seen, each actuator 8420 is provided with an antenna, which antenna is in signal communication with the control unit 8310. In an exemplary embodiment, control unit 8310 can control the actuation of those actuators 8420 so as to position the insertion guide 3900 at the desired position relative to the recipient. That said, in an alternate embodiment, a single antenna can be utilized, such as one mounted on housing 8414, which in turn is connected to a decoding device that outputs a control signal, such as a driver signal based on the decoded RF signal, to the actuators 8420 (as opposed to each actuator having such a device), which control signals can be provided via a wired system/electrical leads extending from housing 8414 to the actuators. Note also that in some alternate embodiments, control unit 8310 is in wired communication with the actuators, either directly or indirectly, and/or is in wired communication with the decoding device located in the housing 8414. Any arrangement that can enable control of the robot apparatus in general, and the actuators thereof in particular, via control unit 8310 can be utilized in at least some exemplary embodiments.

Note also that while the embodiment depicted in FIG. 37 is such that the actuators 8420 must actuate so as to extend the intracochlear portion of the insertion guide into the cochlea, in an alternate embodiment, as noted above, the insertion guide can be mounted on a rail system or the like, wherein a cylindrical actuator or the like pushes the insertion guide in a linear manner into the cochlea and withdrawals the insertion guide in the linear manner from the cochlea. In an exemplary embodiment, this actuator apparatus can enable one degree of freedom movements of the insertion guide, while in other embodiments, this actuator apparatus can enable two or three or four or five or six degrees of freedom. Indeed, in an exemplary embodiment, this actuator apparatus can enable movement only in a linear direction, but can enable rotation of the insertion guide about the longitudinal axis thereof. Any arrangement of actuator assemblies that will enable the insertion guide to be positioned relative to the cochlea and/or inserted into the cochlea via robotic positioning thereof can be utilized in at least some exemplary embodiments.

Any control unit and/or test unit or the like disclosed herein can be a personal computer programs was to execute one or more or all of the functionalities associated there with are the other functionalities disclosed herein. In an exemplary embodiment, any control unit and/or test unit or the like can be a dedicated circuit assembly configured so as to execute one or more or all of the functionalities associated there with or the other functionalities disclosed therein. In an exemplary embodiment, and the control unit and/or test unit or the like disclosed herein can be a processor or the like or otherwise can be a programmed processor.

FIG. 38 depicts another exemplary embodiment, as seen. FIG. 38 presents such an exemplary embodiment, with the links between the antennas removed for clarity. Testing system 4044 detailed shown in signal communication with control unit 8310. In this exemplary embodiment, system 4044 corresponds to that detailed above vis-à-vis determining anomalous electrode location with the exception that it is entirely divorced from the insertion guide, save for the communication between system 4044 and the control unit 8310, to the extent such is relevant for the purposes of discussion, where control unit 8310 is in signal communication with one or more of the assemblies of the robot apparatus, such as the actuator assembly 7720. Here, during insertion, and/or prior to insertion and/or after insertion, the system 4044 monitors or otherwise measures electrical phenomenon detailed herein and communicates those measurements and/or the analysis thereof to control unit 8310, which analyzes those signals and develops a control regime for electrode array insertion and/or electrode array positioning based on those signals. Note also that in some exemplary embodiments, the system 4044 can have multiple measurement electrodes and/or signal generators/sources of acoustic signal generation, some of which are part of the robot apparatus, and some of which are separate from the robot apparatus, all of which are part of system 4044. Alternatively, these various components of the system 4044 can communicate with test unit 3960. Such can have utilitarian value with respect to a scenario where measurements are first taken prior to placing the electrode array near the cochlea and after inserting the electrode array into the cochlea, where it is undesirable to have the insertion guide and/or electrode array support proximate the cochlea. Any device, system, and/or method that will enable controlled movement of the electrode array relative to the cochlea based on electrical phenomenon associated with the recipient/based on electrical characteristics associated with the recipient can be utilized in at least some exemplary embodiments.

Again, the test unit and the system 4044 can be one and the same in some embodiments, and in some embodiments, functionality can be bifurcated between the two as separate units. Indeed, 4044 in FIG. 38 can be a proxy for the control unit and/or the test units detailed above.

In view of the above, it can be seen that some embodiments provide for the automatic detection of a fold over array, a dislocation, bowing or buckling, or other phenomenon, in patients with cochlear implants in an objective manner, and such can provide an automated method for identifying the affected area. Again, the teachings herein can be executed without or in addition to medical imaging tests (e.g., CT scan, X-ray, etc.), or otherwise requiring the recipient/patient to be exposed to radiation during the process of obtaining medical images, and/or subsequent analysis by an expert to assess the correct insertion of the electrode holder and/or measuring neuronal activation after stimulation. In some embodiments, the teachings herein can be executed with methods to attempt to detect neural activation, and can still provide the above reliability in a scenario where there is no neuronal response due to several causes not related to the orientation of the array.

Any method action and/or functionality disclosed herein where the art enables such corresponds to a disclosure of a code from a machine learning algorithm and/or a code of a machine learning algorithm and/or a product of machine learning for execution of such. Still as noted above, in an exemplary embodiment, the code need not necessarily be from a machine learning algorithm, and in some embodiments, the code is not from a machine learning algorithm or the like. That is, in some embodiments, the code results from traditional programming. Still, in this regard, the code can correspond to a trained neural network. In an embodiment, the trained neural network can be utilized to provide (or extract therefrom) an algorithm that can be utilized separately from the trainable neural network. In one embodiment, there is a path of training that constitutes a machine learning algorithm starting off untrained, and then the machine learning algorithm is trained and “graduates,” or matures into a usable code—code of trained machine learning algorithm. With respect to another path, the code from a trained machine learning algorithm is the “offspring” of the trained machine learning algorithm (or some variant thereof, or predecessor thereof), which could be considered a mutant offspring or a clone thereof. That is, with respect to this second path, in at least some exemplary embodiments, the features of the machine learning algorithm that enabled the machine learning algorithm to learn may not be utilized in the practice some of the method actions, and thus are not present the ultimate system. Instead, only the resulting product of the learning is used.

And to be clear, in an exemplary embodiment, there are products of machine learning algorithms (e.g., the code from the trained machine learning algorithm) that are included in any one or more of the systems/subsystems detailed herein, that can be utilized to analyze any of the data obtained or otherwise available disclosed above that can be utilized or otherwise is utilized to evaluate the data obtained herein. This can be embodied in software code and/or in computer chip(s) that are included in the system(s).

An exemplary system includes an exemplary device/devices that can enable the teachings detailed herein, which in at least some embodiments can utilize automation. That is, an exemplary embodiment includes executing one or more or all of the methods and/or functionalities detailed herein and variations thereof, at least in part, in an automated or semiautomated manner using any of the teachings herein. Conversely, embodiments include devices and/or systems and/or methods where automation is specifically prohibited, either by lack of enablement of an automated feature or the complete absence of such capability in the first instance.

Prediction can be represented by an algorithm where circuitry receives the input (embodied in an analogue or a digital signal), where the input suite converts the “physical” input into electronic signals using analog to digital converters for example, or in the case of the input suite corresponding to an Internet server, receives the digital signal from a remote location, and the digital data is stored in a memory and/or received by the electronics. The electronics, which is a result of the machine learning, takes the digital signal and deconstructs the digital signal to evaluate properties, and then, using its “knowledge” from its training, provides an output corresponding to the prediction. By analogy, the operation is analogous to how a human being “predicts” how he or she will function if he or she foregoes a meal for example, or stays up all night, or if he or she drinks 5 cups of coffee in one hour. Past experience informs the future results, the prediction.

The cohort comparator can be a database such as Microsoft™ Access, where the computer automatically matches the data instead of the human matching the data. The results of machine learning and/or a product thereof can be used to perform the automatic matching.

In an exemplary embodiment, the cohort comparator is a computer chip and/or a computer circuit. The cohort comparator can be electronics. In an exemplary embodiment, cohort comparison can be represented by an algorithm where circuitry receives the input (embodied in an analogue or a digital signal), where the input suite converts the “physical” input into electronic signals using analog to digital converters for example, or in the case of the input suite corresponding to an Internet server, receives the digital signal from a remote location, and the digital data is stored in a memory and/or received by the electronics. The electronics takes the digital data and “looks” for certain strings of zeros and ones that correspond to a match with signatures/identifiers linked to prestored data regarding performance capabilities. The data linked to the signatures/identifiers is the cohort identified.

Embodiments can include a system and/or simply an embodiment that includes a non-transitory computer readable medium having recorded thereon, a computer program for executing at least a portion of a method, the computer program including code for executing any one or more of the method actions and/or functionalities detailed herein. Thus, any disclosure herein of a method action or functionality corresponds to a disclosure of a non-transitory computer readable medium having programed thereon code to execute one or more of those actions and also a product to execute one or more of those actions.

Embodiments include any functionality disclosed herein and/or method action disclosed herein being executed by a computer chip, a processor, software, logic circuitry and/or electronics, and all are not mutually exclusive. Any circuit that can enable the teachings herein can be used providing that the art enables such. Thus, in the interests of textual economy, and disclosure herein of a functionality of an article of manufacture corresponds to any one or more of the aforementioned structures being configured to execute such and otherwise for such, and the same is for any method action disclosed herein, where any such action corresponds to a disclosure of any one or more of the aforementioned structures being configured to execute such and otherwise for such.

Any disclosure herein of a processor corresponds to a disclosure in an embodiment of a non-processor device or a combined processor-non-processor device where the non-processor is a result of machine learning. Embodiments can include a link from the cloud to a clinic to pass information back and forth, enabling the remote processing noted above and/or enabling the obtaining of additional data for retraining purposes. Information can be uploaded to the cloud to the clinic, where the information can be analyzed. Another exemplary system includes a smart device, such as a smart phone or tablet, etc., that is running a purpose built application to implement some of the teachings detailed herein. This can be used by the clinician, and can contain at least the front end portions of the systems and devices detailed herein, or otherwise provide the interface portal to the back end. Any disclosure herein of a processor corresponds to a disclosure of a non-processing device, or includes non-processing devices, such as a chip or the like that is a result of a machine learning algorithm or machine learning system, etc.

It is further noted that any disclosure of a device and/or system detailed herein also corresponds to a disclosure of otherwise providing that device and/or system and/or utilizing that device and/or system.

It is also noted that any disclosure herein of any process of manufacturing or providing a device corresponds to a disclosure of a device and/or system that results therefrom. Is also noted that any disclosure herein of any device and/or system corresponds to a disclosure of a method of producing or otherwise providing or otherwise making such. Any functionality of a device disclosed herein corresponds to a disclosure of a method action corresponding to that functionality. Any method action disclosed herein corresponds to a disclosure of a device and/or system for executing such, providing that the art enables such.

An exemplary system includes an exemplary device/devices that can enable the teachings detailed herein, which in at least some embodiments can utilize automation, as will now be described in the context of an automated system. That is, an exemplary embodiment includes executing one or more or all of the methods detailed herein and variations thereof, at least in part, in an automated or semiautomated manner using any of the teachings herein.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

Any function or method action detailed herein corresponds to a disclosure of doing so in an automated or semi-automated manner.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A method, comprising:
obtaining a model of a surface bounding a cavity inside a human during a medical device implantation procedure; and
at least one of confirming a position or repositioning the medical device based on the model of the surface.

2. The method of claim 1, wherein:
the method includes confirming the position of the medical device based on the model of the surface.

3. (canceled)

4. The method of claim 1, wherein:
the cavity is a cranial cavity of the human.

5. The method of claim 1, wherein:
the method includes adjusting the medical device based on the model of the surface.

6. The method of claim 1, wherein:
the cavity is a cochlea of the human.

7. (canceled)

8. The method of claim 1, wherein:
the surface is a lateral wall of a cochlea of the human.

9-12. (canceled)

13. The method of claim 1, wherein:
the cavity is a cavity of the cochleovestibular complex.

14-28. (canceled)

29. A method, comprising:
advancing, as part of an implantation procedure into a human, at least a first portion of an electrode array into a cavity in a human during a first temporal period;
providing information to a computer system, the provided information including spatial based data relating to the electrode array during the implantation procedure of the electrode array into the human;
receiving information based on an evaluation by the computer system of the provided information, the evaluation having used the spatial based data to estimate a feature of the cavity, the received information being an indication of proximity between the electrode array and the feature of the cavity; and
at least one of completing the implantation procedure including leaving the electrode array at its current location based on the received information or moving the electrode array based on the received information.

30. The method of claim 29, wherein:
the electrode array includes a tip portion at a distal end of the electrode array; and
the action of advancing includes moving the tip portion so that the tip portion is in contact with the wall of the cavity for at least about 70% of the distance that the electrode array is inserted into the cavity upon completion of the implantation procedure.

31. The method of claim 30, wherein the cavity is a cochlea and the wall is a modiolus wall of the cochlea.

32. The method of claim 29, wherein:
at least one of:
(i) the spatial based data includes a plurality of positions of a portion of the electrode array at different temporal locations during the action of advancing; or
(ii) the spatial based data includes a plurality of positions of respective different portions of the electrode array during the action of advancing;
the evaluation used the plurality of positions of the portion and/or the plurality of positions of respective different portions to develop a virtual model of the wall; and
the received indication is based on the distance between some portion of the electrode array and the virtual model, the some portion corresponding to the different portions or another portion of the electrode array.

33. The method of claim 29, wherein:
the spatial based data includes a plurality of positions of a first portion of the electrode array at different temporal locations during the action of advancing and/or a plurality of positions of respective different second portions of the electrode array during the action of advancing;
the method includes developing a digital estimate of the wall based on one or both of the plurality of positions by treating the one or both of the plurality of positions as latent variables indicative of position of the wall; and
the received indication is based on at least one of:
the distance between another portion of the electrode array different from the first portion of the electrode array and the digital estimate of the wall at a temporal location after the different temporal locations; or
the distance between one or more of the first portion and/or the another portion and/or one or more of the second portions and the digital estimate of the wall.

34. The method of claim 33, wherein the method includes:
moving the electrode array based on the received information;
providing second information after the action of moving, the provided second information including spatial based data relating to the electrode array during the implantation procedure of the electrode array into the human after the action of moving the electrode array;
receiving second information after the action of providing second information, the received second information being based on a second evaluation by the computer system of the provided second information, the second evaluation having used the estimated location of the wall and the spatial based data of the second information to estimate a location of the electrode array relative to the wall;
advancing the array and then moving the electrode array backwards based on the received second information;
receiving a third information to leave the electrode array at the location where it was moved backwards; and
leaving the electrode array at the location where it was moved backwards based on the third information.

35. (canceled)

36. A method, comprising:
obtaining data indicative of a plurality of different spatial locations of a first portion(s) of a medical device, the plurality of different spatial locations being locations during an insertion process of the first portion(s) into a human;
analyzing the data; and
at least one of:
determining, based on the analysis, a location of a surface of a cavity in the human; or
determining, based on the analysis, a spatial relationship between the surface of the cavity and a second portion, the second portion being one of another portion of the medical device located away from the first portion(s) of the medical device or a portion of another medical device.

37. The method of claim 36, wherein:
there are at least five different spatial locations in the obtained data.

38. The method of claim 36, wherein:
there are at least nine different spatial locations in the obtained data.

39. The method of claim 36, wherein:
the method includes determining the location of the surface of the cavity in the human based on the analysis; and
the surface is a surface that is not readily imaged with a standard X-Ray or standard CT scan.

40. The method of claim 36, wherein:
the method includes determining the location of the surface of the cavity in the human based on the analysis; and
the surface is a surface that is not readily imaged with a cone beam CT scan.

41. The method of claim 36, wherein:
the cavity is a cochlea; and
the surface is a surface of a modiolus wall of the cochlea.

42. The method of claim 36, wherein:
there are at least three different spatial locations in the obtained data;
the first portion(s) of the medical device is a first portion of the medical device;
the spatial locations correspond to increasing depth insertion of the first portion into a cochlea of the person, the cochlea corresponding to the cavity;
the method includes determining the location of the surface of the cavity in the human based on the analysis;
the analysis includes using the at least three different spatial locations as a proxy for the surface; and
the data indicative of a plurality of different spatial locations is based on imaging obtained during the insertion process.

43-50. (canceled)
